SEQUENCE LISTING (4 sequences)

SEQ ID NO: 1 - DNA, 15 bp, synthetic construct
  source 1..15: mol_type = other DNA
tacagactttggaat

SEQ ID NO: 2 - DNA, 15 bp, synthetic construct
  source 1..15: mol_type = other DNA
atgactgaaacctta

SEQ ID NO: 3 - DNA, 18 bp, synthetic construct
  source 1..18: mol_type = other DNA
tacagactagtttggaat

SEQ ID NO: 4 - DNA, 18 bp, synthetic construct
  source 1..18: mol_type = other DNA
atgactgatcaaacctta

(19) United States
(12) Patent Application Publication
(10) Pub. No.: US 2024/0177806 A1
(43) Pub. Date: May 30, 2024

(54) DEEP LEARNING BASED METHOD FOR DIAGNOSING AND PREDICTING CANCER TYPE USING CHARACTERISTICS OF CELL-FREE NUCLEIC ACID

(71) Applicant: GC GENOME CORPORATION, Gyeonggi-do (KR)

(72) Inventors: Chang-Seok KI, Gyeonggi- do (KR); Eun-Hae CHO, Gyeonggi- do (KR); Junnam LEE, Gyeonggi- do (KR); Tae-Rim LEE, Gyeonggi- do (KR)

(21) Appl. No.: 18/164,387

(22) Filed: Feb. 3, 2023

(30) Foreign Application Priority Data
Nov. 29, 2022 (KR) 10-2022-0162988

(51) Int. Cl. G16B 40/00; G16B 20/20; G16B 30/10; G16H 50/20

(52) U.S. Cl. CPC G16B 40/00; G16B 20/20; G16B 30/10; G16H 50/20

(57) ABSTRACT

Disclosed are a method for diagnosing cancer and predicting a cancer type using characteristics of cell-free nucleic acids. More preferably, disclosed are an artificial intelligence-based method for diagnosing cancer and predicting a cancer type using characteristics of cell-free nucleic acids, the method including extracting nucleic acids from a biological sample to obtain sequence information (reads), acquiring information associated with the distribution of cancer-specific single nucleotide variants (regional mutation density, RMD), the frequency of cancer-specific single nucleotide variants depending on types of mutations (mutation signature), the end sequence motif frequency of nucleic acid fragments, and the size of nucleic acid fragments based on the aligned reads, inputting the information to an artificial intelligence model, and analyzing integrated output values. The method for diagnosing cancer and predicting a cancer type using the characteristics of cell-free nucleic acid fragments exhibits high sensitivity and accuracy, compared to other methods for diagnosing cancer and predicting cancer types using genetic information of cell-free nucleic acids, and exhibits high sensitivity and accuracy despite low read coverage because it includes analyzing vectorized data, thus being useful.

### CROSS-REFERENCE TO RELATED APPLICATION

The priority under 35 USC § 119 of Korean Patent Application 10-2022-0162988 filed Nov. 29, 2022 is hereby claimed, and the disclosure thereof is hereby incorporated herein by reference, in its entirety, for all purposes.

### SEQUENCE LISTING

This application includes an electronically submitted sequence listing in .xml format. The .xml file contains a sequence listing entitled “665_SeqListing.xml” created on Feb. 3, 2023 and is 4,628 bytes in size. The sequence listing contained in this .xml file is part of the specification and is hereby incorporated by reference herein in its entirety.

### BACKGROUND OF THE INVENTION

### Field of the Invention

The present invention relates to a method for diagnosing cancer and predicting a cancer type using characteristics of cell-free nucleic acids, and more preferably, to an artificial intelligence-based method for diagnosing cancer and predicting a cancer type using characteristics of cell-free nucleic acids, the method including extracting nucleic acids from a biological sample to obtain sequence information (reads), acquiring information associated with the distribution of cancer-specific single nucleotide variants (regional mutation density, RMD), the frequency of cancer-specific single nucleotide variants depending on types of mutations (mutation signature), the end sequence motif frequency of nucleic acid fragments, and the size of nucleic acid fragments based on the aligned reads, inputting the information to an artificial intelligence model, and analyzing integrated output values.

### Description of the Related Art

Cancer in clinical practice is usually performed by tissue biopsy after history examination, physical examination, and clinical evaluation. Cancer diagnosis based on clinical trials is possible only when the number of cancer cells is 1 billion or more and the diameter of the cancer is 1 cm or more. In this case, cancer cells already have the potential to metastasize and at least half thereof have already metastasized. In addition, tissue biopsy is invasive, which disadvantageously causes patients considerable discomfort and is often incompatible with cancer therapy. Further, tumor markers for monitoring substances produced directly or indirectly from cancer are used in cancer screening. However, the tumor markers have limited accuracy because more than half of tumor marker screening results indicate normal even in the presence of cancer and tumor marker screening results often indicate positive even in the absence of cancer.

Research on diagnosing cancer through analysis of single nucleotide variants of cell-free DNA is being actively conducted and targeted sequencing with the increased sequencing depth of recurrent mutations frequently found in cancer has been widely used (Chabon J. J. et al., nature, Vol. 580, pp. 245-251, 2020). However, analysis of more types of variants using whole-genome sequencing (WGS) data of cell-free DNA has recently been found to be more sensitive than targeted sequencing although the sequencing depth is lower (Zviran A et al., Nat Med, Vol. 26, pp. 1114-1124, 2020).

However, in the current technology, [symbol] cell-free DNA WGS cannot be used for diagnosis of cancer due to the problem of accuracy of mutation detection in cell-free DNA WGS, and WGS of cell-free DNA was used only for cancer recurrence monitoring which tracks only the filtered corresponding variant when mutation information of the patient is obtained through tumor tissue WGS (Zviran A et al., Nat Med, Vol. 26, pp. 1114-1124, 2020). That is, cell-free DNA WGS cannot be used for cancer diagnosis due to the absence of an effective filtering method although it is effective to use cell-free DNA WGS for cancer diagnosis.

Meanwhile, the mutation rate in cancer differs by region of the genome, and furthermore, the mechanism by which mutation occurs and the pattern of mutation accumulation vary depending on the type of cancer. Based thereon, it has been reported that the type of cancer can be detected using regional mutation density and mutation signature in cancer tissues (Jia Wei et al., Nat. Communications, Vol. 11, no. 728, 2020). However, this case results from exploration of the theoretical possibility after cancer diagnosis and cancer type detection had already been completed through surgery and was not applied to cancer diagnosis through cell-free DNA WGS.

In addition, a method for diagnosing cancer using the ends of cell-free nucleic acids has been known (US 2020-0199656 A1), but has a drawback of low accuracy.

In addition, there are various patents (KR 10-2017-0185041, KR 10-2017-0144237, and KR 10-2018-0124550) that utilize artificial neural networks in the life sciences, but methods for predicting cancer types by analyzing mutations based on WGS sequencing information of cell-free DNA in blood (cell-free DNA, cfDNA) are insufficient due to the problem of inaccuracy of cancer-specific mutation discovery.

Accordingly, as a result of extensive efforts to solve the problems and develop methods for diagnosing cancer and predicting cancer types using single nucleotide variants of cell-free nucleic acids with high sensitivity and accuracy, the present inventors found that cancer diagnosis and cancer type prediction with high sensitivity and accuracy can be accomplished by extracting nucleic acids from biological samples to obtain sequence information, obtaining cancer-specific single nucleotide variant information through filtering based on aligned reads, obtaining end sequence motif frequency information of nucleic acid fragments and size information of nucleic acid fragments, inputting the information to each artificial intelligence model, and then integrating and analyzing the output values. Based thereon, the present invention has been completed.

### SUMMARY OF THE INVENTION

Therefore, it is one object of the present invention to provide a method for diagnosing cancer and predicting a cancer type using characteristics of cell-free nucleic acids.

It is another object of the present invention to provide a device for diagnosing cancer and predicting a cancer type using characteristics of cell-free nucleic acids.

It is another object of the present invention to provide a computer-readable storage medium including instructions configured to be executed by a processor for diagnosing cancer and predicting a cancer type by the method described above.

In accordance with one aspect of the present invention, provided is a method for diagnosing cancer and predicting a cancer type, the method including (a) obtaining a sequence information by extracting nucleic acids from a biological sample; (b) aligning the sequence information (reads) with a reference genome database; (c) dividing the reference genome into predetermined bins; (d) obtaining two or more pieces of information selected from the group consisting of cancer-specific single nucleotide variant distribution (regional mutation density, RMD) information, cancer-specific single nucleotide variant frequency (mutation signature) information, end sequence motif frequency information of nucleic acid fragments, and size information of nucleic acid fragments using the aligned reads in predetermined bins; (e) obtaining an output value by inputting the two or more pieces of information to an artificial intelligence model trained to perform cancer diagnosis and cancer type prediction and analyzing the same; (f) determining whether or not cancer develops by comparing the analyzed output value with a cut-off value; and (g) predicting a cancer type through comparison of the output value.

In accordance with another aspect of the present invention, provided is a device for diagnosing cancer and predicting a cancer type, the device including a decoder configured to extract nucleic acids from a biological sample and decode sequence information, an aligner configured to align the decoded sequence with a reference genome database, an input information receiver configured to divide the reference genome into predetermined bins and obtain two or more pieces of information selected from the group consisting of cancer-specific single nucleotide variant distribution (regional mutation density, RMD) information, cancer-specific single nucleotide variant frequency (mutation signature) information, end sequence motif frequency information of nucleic acid fragments, and size information of nucleic acid fragments using the aligned reads in each of predetermined bins, an artificial intelligence model analyzer configured to input the two or more pieces of information to an artificial intelligence model trained to perform cancer diagnosis and cancer type prediction and analyze the information to obtain an output value, a cancer diagnostic unit configured to compare the output value with a cut-off value to determine whether or not cancer develops, and a cancer type predictor configured to predict a cancer type through comparison of the output values.

In accordance with another aspect of the present invention, provided is a computer-readable storage medium including an instruction configured to be executed by a processor for diagnosing cancer and predicting a cancer type through the following steps, including (a) obtaining a sequence information by extracting nucleic acids from a biological sample; (b) aligning the sequence information (reads) with a reference genome database; (c) dividing the reference genome into predetermined bins; (d) obtaining two or more pieces of information selected from the group consisting of cancer-specific single nucleotide variant distribution (regional mutation density, RMD) information, cancer-specific single nucleotide variant frequency (mutation signature) information, end sequence motif frequency information of nucleic acid fragments, and size information of nucleic acid fragments using the aligned reads in predetermined bins; (e) obtaining an output value by inputting the two or more pieces of information to an artificial intelligence model trained to perform cancer diagnosis and cancer type prediction and analyzing the same; (f) determining whether or not cancer develops by comparing the analyzed output value with a cut-off value; and (g) predicting a cancer type through comparison of the output value.

### Effects of the Invention

The method for diagnosing cancer and predicting a cancer type using the characteristics of cell-free nucleic acid fragments according to the present invention exhibits high sensitivity and accuracy, compared to other methods for diagnosing cancer and predicting cancer types using genetic information of cell-free nucleic acids, and exhibits high sensitivity and accuracy in spite of low read coverage because it includes analyzing vectorized data, thus being useful.

### BRIEF DESCRIPTION OF THE DRAWINGS

The above and other objects, features and other advantages of the present invention will be more clearly understood from the following detailed description taken in conjunction with the accompanying drawings, in which:

FIG. 1 is an overall flowchart for determining chromosomal abnormalities using the characteristics of the cell-free nucleic acid of the present invention;

FIG. 2 shows the result of comparative analysis in the cancer diagnosis performance between the DNN model constructed in an embodiment of the present invention and other models, wherein (A) shows the result of comparative analysis in the accuracy of the cancer diagnosis performance, and (B) shows the result of comparative analysis in the cancer type discrimination performance;

FIG. 3 in (A) shows the result of comparative analysis in the cancer diagnosis performance of the DNN model constructed in an embodiment of the present invention for each cancer type, compared to conventional methods, and FIG. 3 in (B) shows the result of comparative analysis for each cancer progression stage therebetween;

FIG. 4 in (A) shows the result of comparative analysis in the cancer type discrimination performance between the DNN model constructed in an embodiment of the present invention and conventional methods and FIG. 4 in (B) shows the result of comparative analysis for each cancer progression stage therebetween;

FIG. 5 illustrates an example of a process of selecting motifs having a difference in expression frequency between healthy subjects and cancer patients, or between respective cancer types according to an embodiment of the present invention;

FIG. 6 is a graph illustrating size distributions of nucleic acid fragments selected according to an embodiment of the present invention;

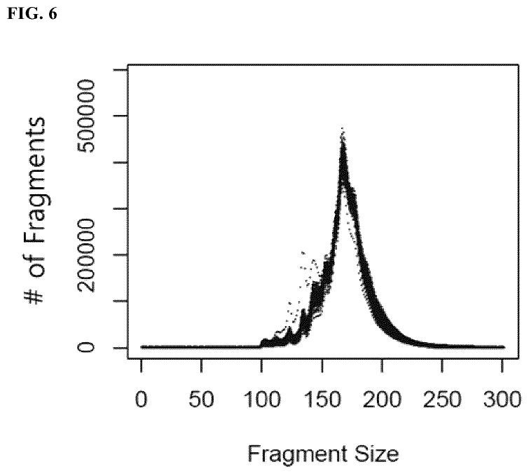

FIG. 7 illustrates an example in which an FEMS table is created from one nucleic acid fragment according to an embodiment of the present invention (left panel) and an example in which the FEMS table is created from all nucleic acid fragments;

FIG. 8 illustrates an example of a FEMS table created by further performing edge summary according to an embodiment of the present invention (left panel) and a result of visualization thereof (right panel);

FIG. 9 illustrates the difference in frequency for each bin of the FEMS table created in an embodiment of the present invention;

FIG. 10 is a schematic diagram illustrating a process of creating a FEMS_Z table according to an embodiment of the present invention;

FIG. 11 illustrates visualization of a FEMS table constructed based on data of healthy subjects and neuroblastoma patients used in an embodiment of the present invention and a FEMS_Z table constructed through standardization;

FIG. 12 shows the result of comparison in the performance between a CNN model using the FEMS table constructed in an embodiment of the present invention and a CNN model using the FEMS_Z table;

FIG. 13 shows the result of actual patient analysis of the CNN model using the FEMS table and the CNN model using the FEMS_Z table constructed in an embodiment of the present invention; and

FIG. 14 shows the result of comparison in the performance between a DNN model, a CNN model, and an ensemble model constructed in an embodiment of the present invention.

### DETAILED DESCRIPTION OF THE INVENTION

Unless defined otherwise, all technical and scientific terms used herein have the same meanings as appreciated by those skilled in the field to which the present invention pertains. In general, the nomenclature used herein is well-known in the art and is ordinarily used.

Terms such as first, second, A, B, and the like may be used to describe various elements, but these elements are not limited by these terms and are merely used to distinguish one element from another. For example, without departing from the scope of the technology described below, a first element may be referred to as a second element and in a similar way, the second element may be referred to as a first element. “And/or” includes any combination of a plurality of related recited items or any one of a plurality of related recited items.

Singular forms are intended to include plural forms as well, unless the context clearly indicates otherwise. It will be further understood that the terms “comprises” and/or “comprising”, when used in this specification, specify the presence of features, numbers, steps, actions, components, parts, or combinations thereof, but do not preclude the presence or addition of one or more other features, numbers, steps, actions, components, parts, or combinations thereof.

Prior to the detailed description of the drawings, it is to be clarified that the classification of components in the present specification is merely made depending on the main function of each component. That is, two or more components described below may be combined into one component or one component may be divided into two or more depending on each more detailed function. In addition, each component to be described below may further perform some or all of the functions of other components in addition to its main function, and some of the main functions of each component may be performed exclusively by other components.

In addition, in implementing a method or operation method, respective steps constituting the method may occur in a different order from a specific order unless the specific order is clearly described in context. That is, the steps may be performed in the specific order, substantially simultaneously, or the reverse order as specified.

The present invention is intended to determine whether or not cancer diagnosis and cancer type prediction with high sensitivity and accuracy are possible by aligning sequencing data obtained from a sample with a reference genome, extracting nucleic acid from biological samples to obtain sequence information, obtaining cancer-specific single nucleotide variant distribution (regional mutation density, RMD) information, cancer-specific single nucleotide variant frequency (mutation signature) information, end sequence motif frequency information of nucleic acid fragments, and size information of nucleic acid fragments based on aligned reads, and inputting the information to a trained artificial intelligence model to diagnose cancer and predict a cancer type.

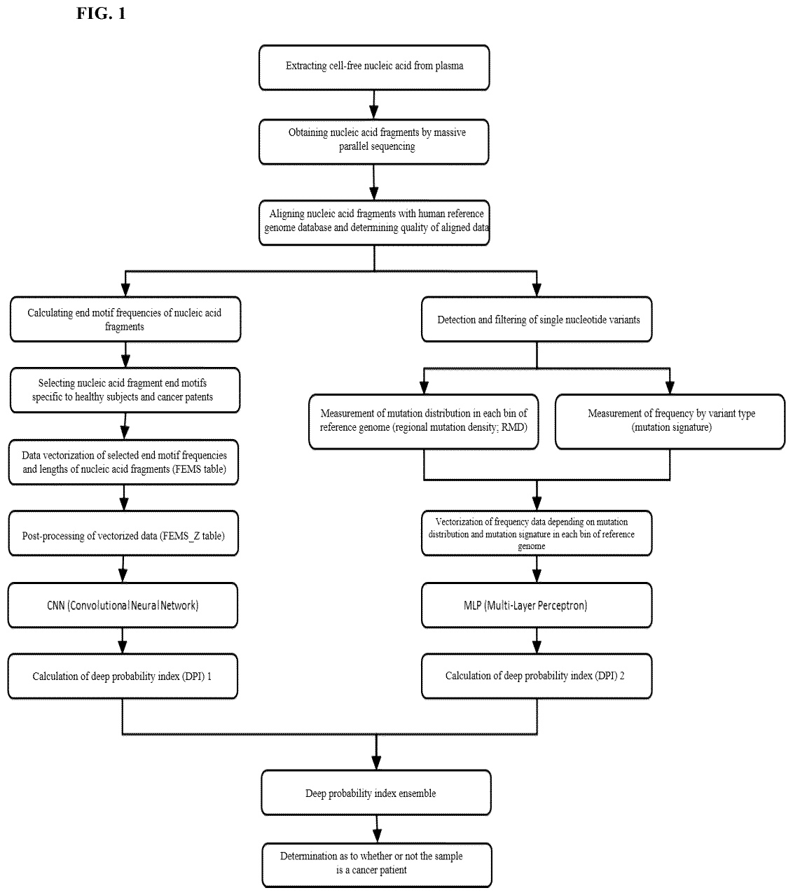

That is, in an embodiment of the present invention, developed is a method including sequencing DNA extracted from blood, aligning the sequencing data with a reference genome, extracting cancer-specific single nucleotide variants through filtering of the aligned reads, dividing the reference genome into predetermined bins, calculating the distribution of single nucleotide variant (regional mutation density, RMD) and the frequency of single nucleotide variant (mutation signature), inputting the distribution and frequency by type of the single nucleotide variant to a first artificial intelligence model trained to perform cancer diagnosis and cancer type prediction to obtain a first output value, acquiring end motif frequencies and sizes of nucleic acid fragments using the aligned reads, generating vectorized data with the end motif frequencies of nucleic acid fragments on the X-axis and the sizes of nucleic acid fragments on the Y-axis to perform post-processing, inputting the data to a second artificial intelligence model trained to perform cancer diagnosis and cancer type prediction to obtain a second output value, integrating and analyzing the first output value and the second output value to obtain a final output value, comparing the final output value with a cut-off value to perform cancer diagnosis, and then determining a type of cancer showing the highest value among the final outputs as the cancer type of the sample (FIG. 1).

In one aspect, the present invention is directed to a method for diagnosing cancer and predicting a cancer type, the method including:
(a) obtaining a sequence information by extracting nucleic acids from a biological sample;
(b) aligning the sequence information (reads) with a reference genome database;
(c) dividing the reference genome into predetermined bins;
(d) obtaining two or more pieces of information selected from the group consisting of cancer-specific single nucleotide variant distribution (regional mutation density, RMD) information, cancer-specific single nucleotide variant frequency (mutation signature) information, end sequence motif frequency information of nucleic acid fragments, and size information of nucleic acid fragments using the aligned reads in predetermined bins;
(e) obtaining an output value by inputting the two or more pieces of information to an artificial intelligence model trained to perform cancer diagnosis and cancer type prediction and analyzing the same;
(f) determining whether or not cancer develops by comparing the analyzed output value with a cut-off value; and
(g) predicting a cancer type through comparison of the output value.

In the present invention, the cancer may be a solid cancer or a blood cancer, is preferably selected from the group consisting of non-Hodgkin lymphoma, Hodgkin lymphoma, acute-myeloid leukemia, acute-lymphoid leukemia, multiple myeloma, head and neck cancer, lung cancer, glioblastoma, colorectal/rectal cancer, pancreatic cancer, breast cancer, ovarian cancer, melanoma, prostate cancer, thyroid cancer, liver cancer, stomach cancer, gallbladder cancer, biliary tract cancer, bladder cancer, small intestine cancer, cervical cancer, cancer of unknown primary, kidney cancer, and mesothelioma, and is most preferably liver cancer or ovarian cancer, but the cancer is not limited thereto.

In the present invention,
step (a) includes:
(a-i) obtaining nucleic acids from a biological sample;
(a-ii) removing proteins, fats, and other residues from the collected nucleic acids using a salting-out method, a column chromatography method, or a bead method to obtain purified nucleic acids;
(a-iii) producing a single-end sequencing or paired-end sequencing library for the purified nucleic acids or nucleic acids randomly fragmented by enzymatic digestion, pulverization, or hydroshearing;
(a-iv) reacting the produced library with a next-generation sequencer; and
(a-v) obtaining sequence information (reads) of the nucleic acids in the next-generation sequencer.

In the present invention, the step (a) of obtaining sequence information may include obtaining the isolated cell-free DNA through whole genome sequencing at a depth of 1 million to 100 million reads.

In the present invention, the biological sample refers to any substance, biological fluid, tissue or cell obtained from or derived from an individual, and examples thereof include, but are not limited to, whole blood, leukocytes, peripheral blood mononuclear peripheral cells, leukocyte buffy coat, blood including plasma and serum, sputum, tears, mucus, nasal washes, nasal aspirates, breath, urine, semen, saliva, peritoneal washings, pelvic fluids, cyst fluids, meningeal fluid, amniotic fluid, glandular fluid, pancreatic fluid, lymph fluid, pleural fluid, nipple aspirate, bronchial aspirate, synovial fluid, joint aspirate, organ secretions, cells, cell extracts, semen, hair, saliva, urine, oral cells, placenta cells, cerebrospinal fluid, and mixtures thereof.

As used herein, the term “reference population” refers to a reference group that is used for comparison like a reference genome database and refers to a population of subjects who do not currently have a specific disease or condition. In the present invention, the reference nucleotide sequence in the reference genome database of the reference population may be a reference chromosome registered with public health institutions such as the NCBI.

In the present invention, the nucleic acid in step (a) may be cell-free DNA, more preferably circulating tumor DNA, but is not limited thereto.

In the present invention, the next-generation sequencer may be used for any sequencing method known in the art. Sequencing of nucleic acids isolated using the selection method is typically performed using next-generation sequencing (NGS). Next-generation sequencing includes any sequencing method that determines the nucleotide sequence either of each nucleic acid molecule or of a proxy cloned from each nucleic acid molecule so as to be highly similar thereto (e.g., 105 or molecules more are sequenced simultaneously). In one embodiment, the relative abundance of nucleic acid species in the library can be estimated by counting the relative number of occurrences of the sequence homologous thereto in data produced by sequencing experimentation. Next-generation sequencing is known in the art, and is described, for example, in Metzker, M. (2010), Nature Biotechnology Reviews 11:31-46, which is incorporated herein by reference.

Platforms for next-generation sequencing include, but are not limited to, the FLX System genome sequencer (GS) from Roche/454, the Illumina/Solexa genome analyzer (GA), the Support Oligonucleotide Ligation Detection (SOLID) system from Life/APG, the G. 007 system from Polonator, the HelioScope gene-sequencing system from Helicos Biosciences, and the PacBio RS system from Pacific Biosciences.

In the present invention, the alignment in step (b) may be performed using the BWA algorithm and the Hg19 sequence, but is not limited thereto.

In the present invention, the BWA algorithm may include BWA-ALN, BWA-SW or Bowtie2, but is not limited thereto.

In the present invention, the length of the sequence information (reads) in step (b) is 5 to 5,000 bp, and the number of sequence information (reads) that are used may be 5,000 to 5 million, but the present invention is not limited thereto.

In the present invention, the bin in step (c) may be used without limitation as long as the artificial intelligence model can be trained and it is preferably 100 kb to 10 Mb, more preferably 500 kb to 5 Mb, most preferably, 1 Mb, but is not limited thereto.

In the present invention, the cancer-specific single nucleotide variant in step (d) may be obtained by detecting single nucleotide variants, followed by filtering and extraction.

In the present invention, any method may be used as the filtering without limitation as long as it is capable of discriminating between single nucleotide variants occurring in normal subjects and single nucleotide variants occurring specifically in cancer, and preferably the filtering includes extracting single nucleotide variants having a read depth of 3 or more and a mean sequencing quality of 30 or more, but is not limited thereto.

In the present invention, the variant region means an area where a single nucleotide variant actually exists and the read depth of 3 or more in the variant region means that the number of reads aligned in the corresponding area is 3 or more.

In the present invention, the filtering may further include removing artifacts and germline mutations generated during the sequencing process and the removing includes, but is not limited to, removing at least one variant selected from the group consisting of:
i) a variant detected in only one of read pairs;
ii) two or more variants detected at a position;
iii) a variant in which normal bases are not detected at each position; and
iv) a variant detected in a normal subject database.

In the present invention, the normal subject database can be used without limitation as long as it includes nucleotide sequence variation information of a normal subject, and is preferably a database that includes cfDNA WGS data of normal subjects, WGS data of tissue samples, or the like, more preferably a public database such as dbSNP, 1000 Genome, Hapmap, ExAC, Gnomad, or the like, but is not limited thereto.

In the present invention, the calculating the distribution information of the cancer-specific single nucleotide variant (regional mutation density, RMD) in step (d) may be performed by a method including the following steps:
(i) calculating the number of single nucleotide variants extracted for each of bins excluding bins in which no variants are detected above the cut-off value of the entire sample; and
(ii) dividing the calculated number by the total number of variants for each bin, following by normalization.

In the present invention, any value may be used as the cut-off value without limitation as long as it can significantly detect the extracted single nucleotide variants, preferably 40 to 60%, more preferably 45 to 55%, most preferably 50%, but is not limited thereto.

In the present invention, the bin excluding the bins in which no variant is detected above the cut-off value of the entire sample means that, when the cut-off value is 50%, the bin in which the single nucleotide variant extracted from more than 50% of all the samples does not exist is excluded.

In the present invention, the bin may be used without limitation as long as it is a significant bin capable of calculating the distribution of cancer-specific single nucleotide variants, and preferably includes at least one selected from the bins shown in Table 1, but is not limited thereto.

In the present invention, the distribution of single nucleotide variants (regional mutation density, RMD) is used as a meaning similar to background mutation rate, and means a mutation frequency calculated by dividing the entire genome into certain bins.

In the present invention, the distribution of single gene variants for each type of cancer is a quantitative value indicating the density of variants that are present. Cancer single nucleotide variants are not evenly distributed in the human genome. The amount of single nucleotide variants that accumulate varies depending on the whole genome region and the accumulation pattern for each type of cancer also greatly varies. In addition, histone modification (replication time) is the main cause of the distribution of single nucleotide variants by cancer type and the distribution of single nucleotide variants implies the epigenetic characteristics of the cancer type.

The distribution of single nucleotide variants can be a beneficial index for cancer diagnosis and cancer type discrimination because it depends on the entire genome region and the type of cancer. The distribution of single nucleotide variants may be used to determine whether or not the detected variant is located in a region with a high probability of occurrence in the cancer.

In the present invention, the step of calculating the cancer-specific single nucleotide variant frequency (mutation signature) information (d) may be performed by a method including the following steps:
(i) calculating the number of mutations for the type of each of the following mutations;
(1) a mutation in which cytosine (C) is substituted with thymine (T), adenine (A) or guanine (G);
(2) a mutation in which thymine is substituted with cytosine, adenine or guanine;
(3) a mutation in which one base in a 5′ direction is further included in the mutation in (1) or (2);
(4) a mutation in which one base in a 3′ direction is further included in the mutation in (1) or (2); and
(5) a mutation in which one base in the 5′ direction and one base in the 3′ direction are further included in a variant in which adenine, guanine, cytosine, and thymine are substituted with different bases; and
(ii) dividing the sum of the calculated numbers of mutations by the total sum, followed by normalization.

In the present invention, the type of mutation may include at least one selected from the mutations listed in Table 2, but is not limited thereto.

In the present invention, any mutation may be used without limitation as the mutation in the single nucleotide variant type (mutation signature) as long as it is a mutation that causes functional abnormality of genes due to substitution of a normal base with another base, preferably includes at least one selected from the group consisting of C→A, C→G, C→T, T→A, T→C and T→G, but is not limited thereto.

In the present invention, C→A means a detected mutation in which a normal base C is mutated to a variant base A, C→G means a detected mutation in which a normal base C is mutated to a variant base G, and the remaining has the same meaning.

In the present invention, the end sequence motif of nucleic acid fragment in step (d) may be a sequence pattern of 2 to 30 bases at both ends of the nucleic acid fragment.

That is, with respect to a nucleic acid fragment sequenced by paired-end sequencing as shown below, the end motifs of the nucleic acid fragment are “TACA” sequentially read from the 5′ end of the forward strand and “ATTC” sequentially read from the 5′ end of the reverse strand.

Forward strand:
(SEQ ID NO: 1)
5′-TACAGACTTTGGAAT-3′
Reverse strand:
(SEQ ID NO: 2)
3′-ATGACTGAAACCTTA-5′

In the present invention, the frequency of the end sequence motifs of the nucleic acid fragment in step (d) may be correspond to the number of motifs detected in all the nucleic acid fragments.

That is, when the end motif of the nucleic acid fragment is analyzed based on the four bases at both ends (4-mer motif), a combination of the four bases, namely, A, T, G, and C, located at the 1^{st}, 2^{nd}, 3^{rd}, and 4^{th} positions, respectively, is possible and thus motif values of a total of 256 (4*4*4*4) are analyzed.

The count of the number of motifs observed in all of the nucleic acid fragments produced by sequencing is referred to as “motif frequency” and the value calculated by dividing the motif frequency by the total number of nucleic acid fragments produced is referred to as “relative frequency”.

TABLE 3

| | AAAA | AAAC | AAAG | AAAT | AACA | AACC | . . . | TTTT | Row Sum |
|---|---|---|---|---|---|---|---|---|---|
| Forward Strand | 62,639 | 105,142 | 127,299 | 75,485 | 399,505 | 42,583 | . . . | 269,530 | 63,319,687 |
| Reverse Strand | 62,432 | 105,719 | 126,493 | 75,788 | 400,900 | 42,467 | . . . | 269,802 | 63,110,437 |
| Merged | 125,071 | 210,861 | 253,792 | 151,273 | 800,405 | 85,050 | . . . | 539,332 | 126,430,124 |
| End Motif | 0.00099 | 0.00167 | 0.00201 | 0.00120 | 0.00633 | 0.00067 | . . . | 0.00427 | — |
| Relative Freq | | | | | | | | | |

As shown in Table 3 above, the total number of nucleic acid fragments is 126,430,124, the number of nucleic acid fragments analyzed from “AAAA”, the end motif of the nucleic acid fragments is 125, 071, the frequency of the end motif of the nucleic acid fragment, “AAAA”, is 125, 071, and the relative frequency of end motifs of the nucleic acid fragments calculated by dividing the frequency by the total number of nucleic acid fragments is 0.00099.

In the present invention, the size of the nucleic acid fragment in step (d) may correspond to the number of bases from the 5′ end to the 3′ end of the nucleic acid fragment.

For example, the size of the nucleic acid fragment analyzed from SEQ ID NOS: 1 and 2 is 15.

In the present invention, the size of the nucleic acid fragment may be 1 to 10,000, preferably 10 to 1,000, more preferably 50 to 500, and most preferably 90 to 250, but the present invention is not limited thereto.

In the present invention, the frequency information of the end motifs of the nucleic acid fragment and size information of nucleic acid fragment in step (d) may further include the following steps before inputting the information to the artificial intelligence model:
(i) generating vectorized data using the end sequence motif frequency information and size information of the nucleic acid fragments; and
(ii) post-processing the vectorized data.

In the present invention, the vectorized data in step (i) may be expressed by the type of the end motif of the nucleic acid fragment plotted on the X-axis and the size of the nucleic acid fragment plotted on the Y-axis.

That is, assuming that there is one nucleic acid fragment as follows,

Forward strand:
(SEQ ID NO: 3)
5'-TACAGACTAGT...TTGGAAT-3′
Reverse strand:
(SEQ ID NO: 4)
3′-ATGACTGATCA...AACCTTA-5′

Fragment Size: 176

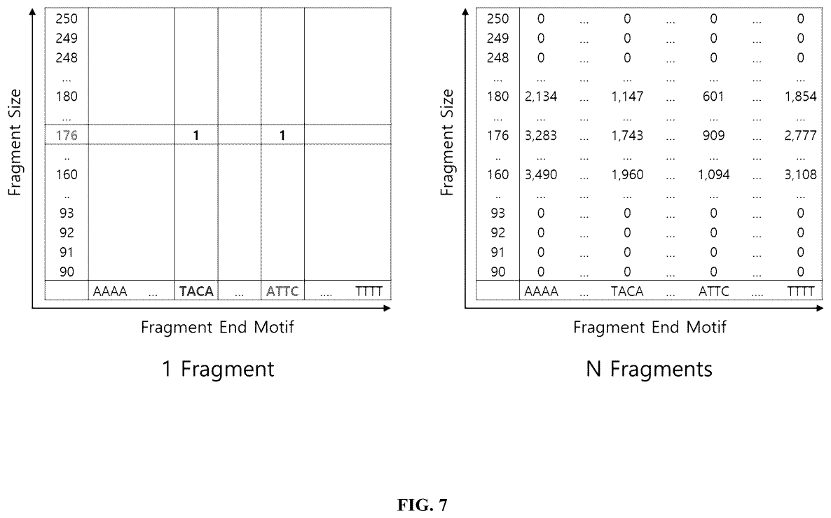

This nucleic acid fragment can be expressed as a two-dimensional vector as shown in the left panel of FIG. 7 and a two-dimensional vector as shown in the right panel of FIG. 7 is generated when this process is performed on an extended entire nucleic acid fragment and accumulated.

In the present invention, the vectorized data may further include the sum of the frequencies for end motifs of nucleic acid fragments and the sum of the frequencies for sizes of nucleic acid fragments.

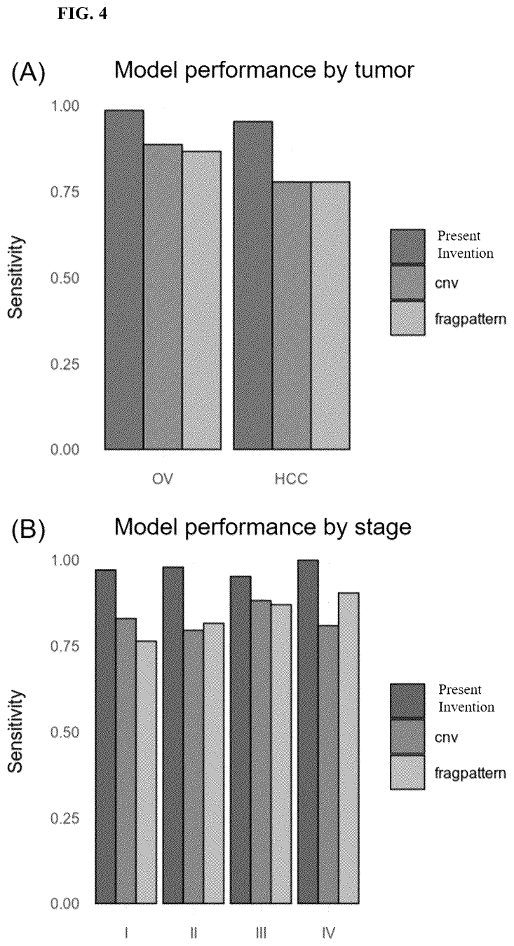

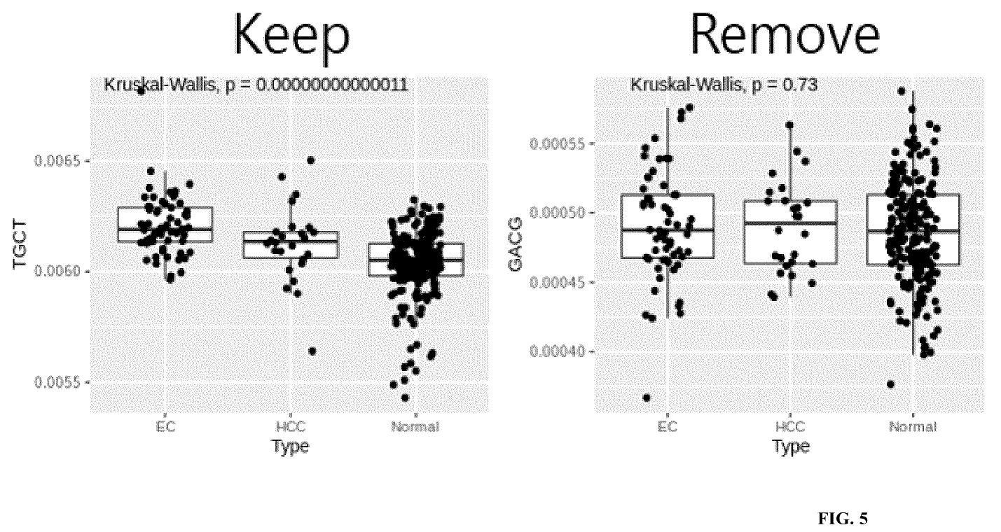

That is, the two-dimensional vector as shown in the left panel of FIG. 5 is generated by further performing an edge summary by adding a column sum four times to the bottom of the two-dimensional vector in FIG. 4 in order to add frequency information for each fragment end motif irrelevant to the fragment size, and adding a row sum four times to the rightmost part of the two-dimensional vector of FIG. 4 in order to add the fragment size information irrelevant to the fragment end motif.

In the present invention, the two-dimensional vector is defined as a fragment end motif frequency and size (FEMS) table. The FEMS table is visualized and the result is shown in the right panel of FIG. 5.

In the present invention, the step (ii) of post-processing the vectorized data may be performed by a method including the following steps:
i) calculating the mean and standard deviation of the frequency of the end motifs of the nucleic acid fragment and size of the nucleic acid fragment in a group of normal subjects;
ii) subtracting the mean of the frequency for the end sequence motif type of each nucleic acid fragment and the size of nucleic acid fragment in the normal group from the frequency for the end sequence motif type of each nucleic acid fragment and the size of nucleic acid fragment in the sample, and dividing the result by the standard deviation of the frequency for each type of motif and size of the nucleic acid fragment to perform Z standardization and thereby obtain a Z-standardized value; and
iii) correcting the Z-normalized value derived in step ii) with the reference value when the Z-normalized value exceeds a reference range.

In the present invention, the reference range is −5 to 5, and the reference value may be −5 or 5, but is not limited thereto.

That is, the conventional FEMS table is characterized in that post-processing for standardization is performed due to the great difference in the distribution of calculated values for each area.

For example, the post-processing may be performed through the following steps:
(a) selecting 99 healthy subjects included in the training data as a Z reference set;
(b) calculating means and standard deviations observed at each position in the FEMS table in the selected Z reference set,
wherein, for example, the mean and standard deviation of values at the position (a) having a nucleic acid fragment size of 180 and having an AAAA motif were calculated in the FEMS table of the Z reference group of the 99 subjects, and defined as Mean_180_AAAA and SD_180_AAAA, respectively;
(C) performing Z standardization using the mean and standard deviation at each position in the FEMS table calculated in the above process, wherein specifically, the frequency value observed at the position having a nucleic acid fragment size of 180 and the AAAA motif is defined as Value_180_AAAA, Z standardization was performed in accordance with the equation of Z_180_AAAA=(Value_180_AAAA−Mean_180_AAAA)/SD_180_AAAA; and
(d) limiting the minimum and maximum ranges of Z standardization values to −5 for Z<−5 and 5 for Z>5, in order to avoid the influence of Z standardization values that do not fall within the normal range (−5 to 5) due to the excessively small standard deviations.

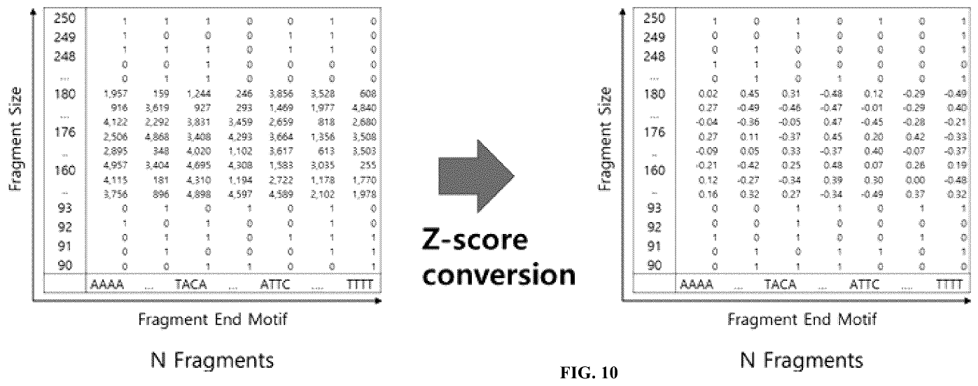

The FEMS_Z table produced through the steps is visualized and the result is shown in FIG. 10.

In the present invention, the vectorized data is preferably a 2D table, but is not limited thereto.

In the present invention, the method may further include, prior to step (c), separating nucleic acid fragments satisfying a mapping quality score from the aligned nucleic acid fragments.

In the present invention, the mapping quality score may vary depending on a desired criterion, but is preferably 15 to 70, more preferably 50 to 70, and most preferably 60.

In the present invention, the two or more pieces of information in step (e) may include cancer-specific single nucleotide variant distribution (regional mutation density, RMD) information and cancer-specific single nucleotide variant frequency (mutation signature) information, or sequence motif frequency information of nucleic acid fragments and size information of nucleic acid fragments, or cancer-specific single nucleotide variant distribution (regional mutation density, RMD) information, cancer-specific single nucleotide variant frequency (mutation signature) information, sequence motif frequency information of nucleic acid fragments, and size information of nucleic acid fragments, but is not limited thereto.

In the present invention, the artificial intelligence model of step (e) may include two or more modules configured to analyze the input two or more pieces of information and output the resulting values.

In the present invention, any model may be used as the module without limitation as long as it is a trained model capable of comparing input information with information of a normal subject to output the result and is preferably selected from the group consisting of K-nearest neighbors, linear regression, logistic regression, support vector machine (SVM), decision trees, random forests, and artificial neural network, but is not limited thereto.

In the present invention, the artificial neural network is selected from the group consisting of a convolutional neural network (CNN), a deep neural network (DNN), a recurrent neural network (RNN), and an autoencoder, but is not limited thereto.

In the present invention, the recurrent neural network is selected from the group consisting of a long-short term memory (LSTM) neural network, a gated recurrent unit (GRU) neural network, a vanilla recurrent neural network, and an attentive recurrent neural network.

In the present invention, the artificial intelligence model in step (e) may further include an output module configured to collect and analyze result values output from each module thereby to output a final result value.

In the present invention, the output module outputs a resulting value selected from the group consisting of a sum, difference, product, mean, logarithm of the product, logarithm of the sum, median, quantile, minimum, maximum, variance, standard deviation, median absolute deviation, and coefficient of variance of a result value output by each module itself or a weighted value.

In the present invention, the output module may be an ensemble model selected from the group consisting of voting, bagging, boosting, and stacking.

In the present invention, the boosting model may be selected from the group consisting of AdaBoost (adaptive boosting), GBM (gradient boosting machine), XGBoost (extra gradient boost) and LightGBM (light gradient boost), but is not limited thereto.

In the present invention, when the module is a DNN and binary classification is trained, the loss function is binary cross entropy represented by Equation 1 below:

$$BCE = - \frac{1}{N} {\sum }_{i = 0}^{N} {y}_{i} \cdot log ( {\hat{y}}_{i} ) + ( 1 - {y}_{i} ) \cdot log ( 1 - {\hat{y}}_{i} ) \qquad Equation 1$$

wherein N is the total number of samples, ŷᵢ is the probability that the model predicts that the i^{th} input value is close to class 1, and yⁱ is the actual class of the i^{th} input value.

In the present invention, when the module is a DNN and multi-class classification is trained, the loss function is categorical cross entropy represented by Equation 2 below:

$$CCE = - \frac{1}{N} {\sum }_{i = 0}^{N} {\sum }_{j = 0}^{J} {y}_{j} \cdot log ( {\hat{y}}_{j} ) + ( 1 - {y}_{j} ) \cdot log ( 1 - {\hat{y}}_{j} ) \qquad Equation 2$$

wherein N is the total number of samples, J is the total number of classes, yⱼ is a value representing the actual class of the sample, wherein yⱼ is 1 provided that the actual class of the sample is j, and yⱼ is 0 provided that the actual class of the sample is not j, and ŷⱼ is the probability with which the sample is predicted to be class j, wherein as ŷⱼ approaches 1, the probability in which the sample is the corresponding class increases.

In the present invention, when the module is a CNN and binary classification is trained, the loss function is represented by Equation 3 below and a loss function performing multi-class classification is represented by Equation 4.

$$Binary classification \qquad Equation 3$$

$$loss ( model ( x ) , y ) = - \frac{1}{n} [ {\sum }_{i = 1}^{n} ( {y}_{i} log ( model ( {x}_{i} ) ) + ( 1 - {y}_{i} ) log ( 1 - model ( {x}_{i} ) ) ) ]$$

Model (xᵢ)=Artificial intelligence model output in response to i^{th} input
y=Actual label value
n=Number of input data

$$Multi - class classification \qquad Equation 4$$

$$loss ( model ( x ) , y ) = - \frac{1}{n} {\sum }_{i = 1}^{n} ( {\sum }_{j = 1}^{c} ( {y}_{ij} {log ( model ( {x}_{i} ) )}_{j} )$$

Model (xᵢ)ⱼ=j^{th} artificial intelligence model output in response to i^{th} input
y=Actual label value
n=Number of input data
c=Number of classes

In the present invention, the binary classification means that the module learns to determine whether or not cancer develops, and multi-class classification means that the module learns to distinguish between two or more cancer types.

In the present invention, the learning of the artificial intelligence model includes the following steps:
i) classifying the generated vector data into training, validation, and test data,
wherein the training data is used to train the artificial intelligence model, the validation data is used to validate hyper-parameter tuning, and the test data is used for the test after optimal model production; and
ii) constructing an optimal artificial intelligence model through hyper-parameter tuning and training; and
iii) comparing the performance of multiple models obtained through hyper-parameter tuning using the validation data and determining the model having the best validation data as the optimal model.

In the present invention, hyper-parameter tuning is a process of optimizing the values of various parameters (the number of convolution layers, the number of dense layers, the number of convolution filters, etc.) constituting the artificial intelligence model. Hyper-parameter tuning is performed using Bayesian optimization and grid search methods.

In the present invention, the internal parameters (weights) of the artificial intelligence model are optimized using predetermined hyper-parameters, and it is determined that the model is over-fit when validation loss starts to increase compared to training loss and then training is stopped.

In the present invention, any value resulting from analysis of the input vectorized data by the artificial intelligence model in step (e) may be used without limitation, as long as it is a specific score or real number, and the value is preferably a deep probability index (DPI), but is not limited thereto.

As used herein, the term “deep probability index” refers to a value expressed as a probability value by adjusting the output of artificial intelligence to a scale of 0 to 1 using for the last layer of the artificial intelligence model, a sigmoid function in the case of binary classification and a SoftMax function in the case of multi-class classification.

In binary classification, training is performed using the sigmoid function such that the DPI is adjusted to 1, provided that cancer develops. For example, when a breast cancer sample and a normal sample are input, training is performed such that the DPI of the breast cancer sample is close to 1.

In multi-class classification, as many DPIs as the number of classes are extracted using the softmax function. The sum of the DPIs is adjusted to 1 and training is performed such that the DPI of the cancer type is actually adjusted to 1. For example, provided that there are three classes, namely, breast cancer, liver cancer, and normal group, when a breast cancer sample is input, training is performed to adjust a DPI of the breast cancer class to about 1.

In the present invention, the resulting output value of step (e) is obtained for each cancer type.

In the present invention, the artificial intelligence model is trained to adjust an output value to about 1 if there is cancer and to adjust an output value to about 0 if there is no cancer. Therefore, performance (training, validation, test accuracy) is measured based on a cut-off value of 0.5. In other words, if the output value is 0.5 or more, it is determined that there is cancer, and if it is less than 0.5, it is determined that there is no cancer.

Here, it will be apparent to those skilled in the art that the cut-off value of 0.5 may be arbitrarily changed. For example, in an attempt to reduce false positives, the cut-off value may be set to be higher than 0.5 as a stricter criterion for determining whether or not there is cancer, and in an attempt to reduce false negatives, the cut-off value may be set to be lower than 0.5 as a weaker criterion for determining that there is cancer.

Most preferably, the cut-off value can be set by applying unseen data (data containing a solution that is different from that encountered during training) using the trained artificial intelligence model and determining the probability of the DPI.

In the present invention, the step (g) of predicting a cancer type through comparison of the output values includes determining the cancer type showing the highest value among the output result values as the cancer of the sample.

In another aspect, the present invention is directed to a device for diagnosing cancer and predicting a cancer type, the device including:
a decoder configured to extract nucleic acids from a biological sample and decode sequence information,
an aligner configured to align the decoded sequence with a reference genome database,
an input information receiver configured to divide the reference genome into predetermined bins and obtain two or more pieces of information selected from the group consisting of single nucleotide variant distribution (regional mutation density, RMD) information, single nucleotide variant frequency (mutation signature) information, end sequence motif frequency information of nucleic acid fragments, and size information of nucleic acid fragments using the aligned reads in each of predetermined bins;
an artificial intelligence model analyzer configured to input the two or more pieces of information to an artificial intelligence model trained to perform cancer diagnosis and cancer type prediction and analyze the information to obtain an output value;
a cancer diagnostic unit configured to compare the output value with a cut-off value to determine whether or not cancer develops; and
a cancer type predictor configured to predict a cancer type through comparison of the output values.

In the present invention, the decoder may include a nucleic acid injector configured to inject the nucleic acid extracted from an independent device, and a sequence information analyzer configured to analyze the sequence information of the injected nucleic acid, preferably an NGS analyzer, but is not limited thereto.

In the present invention, the decoder may receive and decode sequence information data generated in an independent device.

In the present invention, the device may be a computer system including a processor, a memory, and a storage device, but is not limited thereto.

In another aspect, the present invention is directed to a computer-readable storage medium including an instruction configured to be executed by a processor for diagnosing cancer and predicting a cancer type through the following steps including:
(a) obtaining a sequence information by extracting nucleic acids from a biological sample;
(b) aligning the sequence information (reads) with a reference genome database;
(c) dividing the reference genome into predetermined bins;
(d) obtaining two or more pieces of information selected from the group consisting of cancer-specific single nucleotide variant distribution (regional mutation density, RMD) information, cancer-specific single nucleotide variant frequency (mutation signature) information, end sequence motif frequency information of nucleic acid fragments, and size information of nucleic acid fragments using the aligned reads in predetermined bins;
(e) obtaining an output value by inputting the two or more pieces of information to an artificial intelligence model trained to perform cancer diagnosis and cancer type prediction and analyzing the same;
(f) determining whether or not cancer develops by comparing the analyzed output value with a cut-off value; and
(g) predicting a cancer type through comparison of the output value.

In another aspect, the method according to the present disclosure may be implemented using a computer. In one embodiment, the computer includes one or more processors coupled to a chipset. In addition, a memory, a storage device, a keyboard, a graphics adapter, a pointing device, a network adapter and the like are connected to the chipset. In one embodiment, the performance of the chipset is acquired by a memory controller hub and an I/O controller hub. In another embodiment, the memory may be directly coupled to a processor instead of the chipset. The storage device is any device capable of maintaining data, including a hard drive, compact disc read-only memory (CD-ROM), DVD, or other memory devices. The memory is concerned with data and instructions used by the processor. The pointing device may be a mouse, track ball or other type of pointing device, and is used in combination with a keyboard to transmit input data to a computer system. The graphics adapter presents images and other information on a display. The network adapter is connected to the computer system through a local area network or a long distance communication network. However, the computer used herein is not limited to the above configuration, may not have some configurations, may further include additional configurations, and may also be part of a storage area network (SAN), and the computer of the present invention may be configured to be suitable for the execution of modules in the program for the implementation of the method according to the present invention.

The module used herein may mean a functional and structural combination of hardware to implement the technical idea according to the present invention and software to drive the hardware. For example, it is apparent to those skilled in the art that the module may mean a logical unit of a predetermined code and a hardware resource to execute the predetermined code, and does not necessarily mean a physically connected code or one type of hardware.

### EXAMPLE

Hereinafter, the present invention will be described in more detail with reference to examples. However, it will be obvious to those skilled in the art that these examples are provided only for illustration of the present invention, and should not be construed as limiting the scope of the present invention.

### Example 1. Extraction of DNA from Blood and Next-Generation Sequencing to Construct First Artificial Intelligence Model

10 mL of blood was collected from each of 471 normal subjects, 151 ovarian cancer patients, and 131 liver cancer patients, and stored in an EDTA tube. Within 2 hours after blood collection, only the plasma was primarily centrifuged at 1,200 g and 4° C. for 15 minutes, and then the primarily centrifuged plasma was secondarily centrifuged at 16,000 g and 4° C. for 10 minutes to isolate the plasma supernatant excluding the precipitate. Cell-free DNA was extracted from the isolated plasma using a TIANamp Micro DNA Kit (Tiangen), a library preparation process was performed using a MGIEasy cell-free DNA library prep set kit, and then sequencing was performed in a 100 base paired end mode using a DNBseq G400 device (MGI). As a result, about 170 million reads were found to be produced from each sample.

### Example 2. Single Nucleotide Variant Extraction, and Single Nucleotide Variant Distribution and Frequency Feature Extraction by Type

### 2-1. Filtering for Cancer-Specific Variant Extraction

The bam file obtained by aligning the NGS data obtained in Example 1 with the reference genome (hg 19) was processed using the GATK pipeline. To secure the variant profile for each sample, mutations were detected using VarScan (mutation caller).

VarScan mutation detection criteria were very general. Variant calling was performed under general criteria by setting the number of variant reads to at least one, setting the total depth of the variant region to at least 3, setting the mean base quality to at least 30, removing the standard of the minimum variant allele frequency, removing the strand filter, and removing the standard of the VarScan variant P value (variant allele frequency means the ratio of variants as the ratio of the number of reads in which variants were detected to the total number of reads at the variant site).

After all variants that may be cancer-derived variants under generous criteria were detected, artifacts and germline mutations were removed under various criteria. Four methods were used to eliminate variations at inaccurate positions.

First, when sequencing was performed at the position where mutations exist in both the forward read and reverse read of the fragment, if a mutation was found in only one read, it was removed. Second, if there were two or more mutations at one position, the mutations were removed. Third, a variant allele frequency of 1 means that all DNA present in the blood has mutations. Therefore, the removal was performed under the assumption that there was no possibility of tumor-derived mutations.

Fourth, various normal mutation database and blacklist region mutations were removed. The blacklist regions refer to regions with a high misalignment probability, and include regions such as repeats and centromeres. The blacklist regions refer to the areas described in Haley M Ammiya et al., Scientific report Vol. 9, no. 9354, 2019. In addition, in order to remove mutations that are highly likely to be normal, public databases that collect normal mutations were used. dbSNP (https://data.amerigeoss.org/en_KR/dataset/dbsnp), 1000 Genome (https://www.internationalgenome.org/), Hapmap (https://ftp.ncbi.nlm.nih.gov/hapmap/), ExAC (https://gnomad.broadinstitute.org/downloads#exac-variants) and Gnomad (https://gnomad.broadinstitute.org/) databases were used.

In addition, mutations in the cfDNA WGS database of 20,000 normal subjects produced by Green Cross were filtered out because they were unlikely to be tumor-derived mutations. In addition, in the case of the input value of the algorithm for classifying cancer types, the mutations found in the cell-free DNA WGS of 412 normal people in Example 1 were also removed.

### 2-2. Single Nucleotide Variant Distribution Calculation

The whole genome was divided into 1 Mb bins and the regional mutation density (RMD) for each bin was calculated. The distribution of single nucleotide variants (the regional mutation density (RMD) for each bin) in a total of 2726 bins, excluding the bins in which the mutations extracted in Example 2-1 did not exist in more than 50% of the total samples, was used as the input value of the algorithm. The number of variants in each bin was calculated and then was divided by the total number of variants in the 2,726 bins for normalization. Finally, the 2,726 single nucleotide variant distribution features were created and the feature list is shown in Table 1 below.

TABLE 1
Feature list of single nucleotide variant distribution

| | | | | |
|---|---|---|---|---|
| chr1: | chr12: | chr18: | chr3: | chr6: |
| 0-1 Mb | 52 Mb-53 Mb | 36 Mb-37 Mb | 69 Mb-70 Mb | 52 Mb-53 Mb |
| chr1: | chr12: | chr18: | chr3: | chr6: |
| 1 Mb-2 Mb | 53 Mb-54 Mb | 37 Mb-38 Mb | 70 Mb-71 Mb | 53 Mb-54 Mb |
| chr1: | chr12: | chr18: | chr3: | chr6: |
| 2 Mb-3 Mb | 54 Mb-55 Mb | 38 Mb-39 Mb | 71 Mb-72 Mb | 54 Mb-55 Mb |
| chr1: | chr12: | chr18: | chr3: | chr6: |
| 3 Mb-4 Mb | 55 Mb-56 Mb | 39 Mb-40 Mb | 72 Mb-73 Mb | 55 Mb-56 Mb |
| chr1: | chr12: | chr18: | chr3: | chr6: |
| 4 Mb-5 Mb | 56 Mb-57 Mb | 40 Mb-41 Mb | 73 Mb-74 Mb | 56 Mb-57 Mb |
| chr1: | chr12: | chr18: | chr3: | chr6: |
| 5 Mb-6 Mb | 57 Mb-58 Mb | 41 Mb-42 Mb | 74 Mb-75 Mb | 57 Mb-58 Mb |
| chr1: | chr12: | chr18: | chr3: | chr6: |
| 6 Mb-7 Mb | 58 Mb-59 Mb | 42 Mb-43 Mb | 75 Mb-76 Mb | 58 Mb-59 Mb |
| chr1: | chr12: | chr18: | chr3: | chr6: |
| 7 Mb-8 Mb | 59 Mb-60 Mb | 43 Mb-44 Mb | 76 Mb-77 Mb | 61 Mb-62 Mb |
| chr1: | chr12: | chr18: | chr3: | chr6: |
| 8 Mb-9 Mb | 60 Mb-61 Mb | 44 Mb-45 Mb | 77 Mb-78 Mb | 62 Mb-63 Mb |
| chr1: | chr12: | chr18: | chr3: | chr6: |
| 9 Mb-10 Mb | 61 Mb-62 Mb | 45 Mb-46 Mb | 78 Mb-79 Mb | 63 Mb-64 Mb |
| chr1: | chr12: | chr18: | chr3: | chr6: |
| 10 Mb-11 Mb | 62 Mb-63 Mb | 46 Mb-47 Mb | 79 Mb-8 Mb0 | 64 Mb-65 Mb |
| chr1: | chr12: | chr18: | chr3: | chr6: |
| 11 Mb-12 Mb | 63 Mb-64 Mb | 47 Mb-48 Mb | 8 Mb0-81 Mb | 65 Mb-66 Mb |
| chr1: | chr12: | chr18: | chr3: | chr6: |
| 12 Mb-13 Mb | 64 Mb-65 Mb | 48 Mb-49 Mb | 81 Mb-82 Mb | 66 Mb-67 Mb |
| chr1: | chr12: | chr18: | chr3: | chr6: |
| 13 Mb-14 Mb | 65 Mb-66 Mb | 49 Mb-50 Mb | 82 Mb-83 Mb | 67 Mb-68 Mb |
| chr1: | chr12: | chr18: | chr3: | chr6: |
| 14 Mb-15 Mb | 66 Mb-67 Mb | 50 Mb-51 Mb | 83 Mb-84 Mb | 68 Mb-69 Mb |
| chr1: | chr12: | chr18: | chr3: | chr6: |
| 15 Mb-16 Mb | 67 Mb-68 Mb | 51 Mb-52 Mb | 84 Mb-85 Mb | 69 Mb-70 Mb |
| chr1: | chr12: | chr18: | chr3: | chr6: |
| 16 Mb-17 Mb | 68 Mb-69 Mb | 52 Mb-53 Mb | 85 Mb-86 Mb | 70 Mb-71 Mb |
| chr1: | chr12: | chr18: | chr3: | chr6: |
| 17 Mb-18 Mb | 69 Mb-70 Mb | 53 Mb-54 Mb | 86 Mb-87 Mb | 71 Mb-72 Mb |
| chr1: | chr12: | chr18: | chr3: | chr6: |
| 18 Mb-19 Mb | 70 Mb-71 Mb | 54 Mb-55 Mb | 87 Mb-88 Mb | 72 Mb-73 Mb |
| chr1: | chr12: | chr18: | chr3: | chr6: |
| 19 Mb-20 Mb | 71 Mb-72 Mb | 55 Mb-56 Mb | 88 Mb-89 Mb | 73 Mb-74 Mb |
| chr1: | chr12: | chr18: | chr3: | chr6: |
| 20 Mb-21 Mb | 72 Mb-73 Mb | 56 Mb-57 Mb | 89 Mb-90 Mb | 74 Mb-75 Mb |
| chr1: | chr12: | chr18: | chr3: | chr6: |
| 21 Mb-22 Mb | 73 Mb-74 Mb | 57 Mb-58 Mb | 90 Mb-91 Mb | 75 Mb-76 Mb |
| chr1: | chr12: | chr18: | chr3: | chr6: |
| 22 Mb-23 Mb | 74 Mb-75 Mb | 58 Mb-59 Mb | 93 Mb-94 Mb | 76 Mb-77 Mb |
| chr1: | chr12: | chr18: | chr3: | chr6: |
| 23 Mb-24 Mb | 75 Mb-76 Mb | 59 Mb-60 Mb | 94 Mb-95 Mb | 77 Mb-78 Mb |
| chr1: | chr12: | chr18: | chr3: | chr6: |
| 24 Mb-25 Mb | 76 Mb-77 Mb | 60 Mb-61 Mb | 95 Mb-96 Mb | 78 Mb-79 Mb |
| chr1: | chr12: | chr18: | chr3: | chr6: |
| 25 Mb-26 Mb | 77 Mb-78 Mb | 61 Mb-62 Mb | 96 Mb-97 Mb | 79 Mb-8 Mb0 |
| chr1: | chr12: | chr18: | chr3: | chr6: |
| 26 Mb-27 Mb | 78 Mb-79 Mb | 62 Mb-63 Mb | 97 Mb-98 Mb | 8 Mb0-81 Mb |
| chr1: | chr12: | chr18: | chr3: | chr6: |
| 27 Mb-28 Mb | 79 Mb-8 Mb0 | 63 Mb-64 Mb | 98 Mb-99 Mb | 81 Mb-82 Mb |
| chr1: | chr12: | chr18: | chr3: | chr6: |
| 28 Mb-29 Mb | 8 Mb0-81 Mb | 64 Mb-65 Mb | 99 Mb-100 Mb | 82 Mb-83 Mb |
| chr1: | chr12: | chr18: | chr3: | chr6: |
| 29 Mb-30 Mb | 81 Mb-82 Mb | 65 Mb-66 Mb | 100 Mb-101 Mb | 83 Mb-84 Mb |
| chr1: | chr12: | chr18: | chr3: | chr6: |
| 30 Mb-31 Mb | 82 Mb-83 Mb | 66 Mb-67 Mb | 101 Mb-102 Mb | 84 Mb-85 Mb |
| chr1: | chr12: | chr18: | chr3: | chr6: |
| 31 Mb-32 Mb | 83 Mb-84 Mb | 67 Mb-68 Mb | 102 Mb-103 Mb | 85 Mb-86 Mb |
| chr1: | chr12: | chr18: | chr3: | chr6: |
| 32 Mb-33 Mb | 84 Mb-85 Mb | 68 Mb-69 Mb | 103 Mb-104 Mb | 86 Mb-87 Mb |
| chr1: | chr12: | chr18: | chr3: | chr6: |
| 33 Mb-34 Mb | 85 Mb-86 Mb | 69 Mb-70 Mb | 104 Mb-105 Mb | 87 Mb-88 Mb |
| chr1: | chr12: | chr18: | chr3: | chr6: |
| 34 Mb-35 Mb | 86 Mb-87 Mb | 70 Mb-71 Mb | 105 Mb-106 Mb | 88 Mb-89 Mb |
| chr1: | chr12: | chr18: | chr3: | chr6: |
| 35 Mb-36 Mb | 87 Mb-88 Mb | 71 Mb-72 Mb | 106 Mb-107 Mb | 89 Mb-90 Mb |
| chr1: | chr12: | chr18: | chr3: | chr6: |
| 36 Mb-37 Mb | 88 Mb-89 Mb | 72 Mb-73 Mb | 107 Mb-108 Mb | 90 Mb-91 Mb |
| chr1: | chr12: | chr18: | chr3: | chr6: |
| 37 Mb-38 Mb | 89 Mb-90 Mb | 73 Mb-74 Mb | 108 Mb-109 Mb | 91 Mb-92 Mb |
| chr1: | chr12: | chr18: | chr3: | chr6: |
| 38 Mb-39 Mb | 90 Mb-91 Mb | 74 Mb-75 Mb | 109 Mb-110 Mb | 92 Mb-93 Mb |
| chr1: | chr12: | chr18: | chr3: | chr6: |
| 39 Mb-40 Mb | 91 Mb-92 Mb | 75 Mb-76 Mb | 110 Mb-111 Mb | 93 Mb-94 Mb |
| chr1: | chr12: | chr18: | chr3: | chr6: |
| 40 Mb-41 Mb | 92 Mb-93 Mb | 76 Mb-77 Mb | 111 Mb-112 Mb | 94 Mb-95 Mb |
| chr1: | chr12: | chr18: | chr3: | chr6: |
| 41 Mb-42 Mb | 93 Mb-94 Mb | 77 Mb-78 Mb | 112 Mb-113 Mb | 95 Mb-96 Mb |
| chr1: | chr12: | chr18: | chr3: | chr6: |
| 42 Mb-43 Mb | 94 Mb-95 Mb | 78 Mb-78077248 | 113 Mb-114 Mb | 96 Mb-97 Mb |
| chr1: | chr12: | chr19: | chr3: | chr6: |
| 43 Mb-44 Mb | 95 Mb-96 Mb | 0-1 Mb | 114 Mb-115 Mb | 97 Mb-98 Mb |
| chr1: | chr12: | chr19: | chr3: | chr6: |
| 44 Mb-45 Mb | 96 Mb-97 Mb | 1 Mb-2 Mb | 115 Mb-116 Mb | 98 Mb-99 Mb |
| chr1: | chr12: | chr19: | chr3: | chr6: |
| 45 Mb-46 Mb | 97 Mb-98 Mb | 2 Mb-3 Mb | 116 Mb-117 Mb | 99 Mb-100 Mb |
| chr1: | chr12: | chr19: | chr3: | chr6: |
| 46 Mb-47 Mb | 98 Mb-99 Mb | 3 Mb-4 Mb | 117 Mb-118 Mb | 100 Mb-101 Mb |
| chr1: | chr12: | chr19: | chr3: | chr6: |
| 47 Mb-48 Mb | 99 Mb-100 Mb | 4 Mb-5 Mb | 118 Mb-119 Mb | 101 Mb-102 Mb |
| chr1: | chr12: | chr19: | chr3: | chr6: |
| 48 Mb-49 Mb | 100 Mb-101 Mb | 5 Mb-6 Mb | 119 Mb-120 Mb | 102 Mb-103 Mb |
| chr1: | chr12: | chr19: | chr3: | chr6: |
| 49 Mb-50 Mb | 101 Mb-102 Mb | 6 Mb-7 Mb | 120 Mb-121 Mb | 103 Mb-104 Mb |
| chr1: | chr12: | chr19: | chr3: | chr6: |
| 50 Mb-51 Mb | 102 Mb-103 Mb | 7 Mb-8 Mb | 121 Mb-122 Mb | 104 Mb-105 Mb |
| chr1: | chr12: | chr19: | chr3: | chr6: |
| 51 Mb-52 Mb | 103 Mb-104 Mb | 8 Mb-9 Mb | 122 Mb-123 Mb | 105 Mb-106 Mb |
| chr1: | chr12: | chr19: | chr3: | chr6: |
| 52 Mb-53 Mb | 104 Mb-105 Mb | 9 Mb-10 Mb | 123 Mb-124 Mb | 106 Mb-107 Mb |
| chr1: | chr12: | chr19: | chr3: | chr6: |
| 53 Mb-54 Mb | 105 Mb-106 Mb | 10 Mb-11 Mb | 124 Mb-125 Mb | 107 Mb-108 Mb |
| chr1: | chr12: | chr19: | chr3: | chr6: |
| 54 Mb-55 Mb | 106 Mb-107 Mb | 11 Mb-12 Mb | 125 Mb-126 Mb | 108 Mb-109 Mb |
| chr1: | chr12: | chr19: | chr3: | chr6: |
| 55 Mb-56 Mb | 107 Mb-108 Mb | 12 Mb-13 Mb | 126 Mb-127 Mb | 109 Mb-110 Mb |
| chr1: | chr12: | chr19: | chr3: | chr6: |
| 56 Mb-57 Mb | 108 Mb-109 Mb | 13 Mb-14 Mb | 127 Mb-128 Mb | 110 Mb-111 Mb |
| chr1: | chr12: | chr19: | chr3: | chr6: |
| 57 Mb-58 Mb | 109 Mb-110 Mb | 14 Mb-15 Mb | 128 Mb-129 Mb | 111 Mb-112 Mb |
| chr1: | chr12: | chr19: | chr3: | chr6: |
| 58 Mb-59 Mb | 110 Mb-111 Mb | 15 Mb-16 Mb | 129 Mb-130 Mb | 112 Mb-113 Mb |
| chr1: | chr12: | chr19: | chr3: | chr6: |
| 59 Mb-60 Mb | 111 Mb-112 Mb | 16 Mb-17 Mb | 130 Mb-131 Mb | 113 Mb-114 Mb |
| chr1: | chr12: | chr19: | chr3: | chr6: |
| 60 Mb-61 Mb | 112 Mb-113 Mb | 17 Mb-18 Mb | 131 Mb-132 Mb | 114 Mb-115 Mb |
| chr1: | chr12: | chr19: | chr3: | chr6: |
| 61 Mb-62 Mb | 113 Mb-114 Mb | 18 Mb-19 Mb | 132 Mb-133 Mb | 115 Mb-116 Mb |
| chr1: | chr12: | chr19: | chr3: | chr6: |
| 62 Mb-63 Mb | 114 Mb-115 Mb | 19 Mb-20 Mb | 133 Mb-134 Mb | 116 Mb-117 Mb |
| chr1: | chr12: | chr19: | chr3: | chr6: |
| 63 Mb-64 Mb | 115 Mb-116 Mb | 20 Mb-21 Mb | 134 Mb-135 Mb | 117 Mb-118 Mb |
| chr1: | chr12: | chr19: | chr3: | chr6: |
| 64 Mb-65 Mb | 116 Mb-117 Mb | 21 Mb-22 Mb | 135 Mb-136 Mb | 118 Mb-119 Mb |
| chr1: | chr12: | chr19: | chr3: | chr6: |
| 65 Mb-66 Mb | 117 Mb-118 Mb | 22 Mb-23 Mb | 136 Mb-137 Mb | 119 Mb-120 Mb |
| chr1: | chr12: | chr19: | chr3: | chr6: |
| 66 Mb-67 Mb | 118 Mb-119 Mb | 23 Mb-24 Mb | 137 Mb-138 Mb | 120 Mb-121 Mb |
| chr1: | chr12: | chr19: | chr3: | chr6: |
| 67 Mb-68 Mb | 119 Mb-120 Mb | 24 Mb-25 Mb | 138 Mb-139 Mb | 121 Mb-122 Mb |
| chr1: | chr12: | chr19: | chr3: | chr6: |
| 68 Mb-69 Mb | 120 Mb-121 Mb | 28 Mb-29 Mb | 139 Mb-140 Mb | 122 Mb-123 Mb |
| chr1: | chr12: | chr19: | chr3: | chr6: |
| 69 Mb-70 Mb | 121 Mb-122 Mb | 29 Mb-30 Mb | 140 Mb-141 Mb | 123 Mb-124 Mb |
| chr1: | chr12: | chr19: | chr3: | chr6: |
| 70 Mb-71 Mb | 122 Mb-123 Mb | 30 Mb-31 Mb | 141 Mb-142 Mb | 124 Mb-125 Mb |
| chr1: | chr12: | chr19: | chr3: | chr6: |
| 71 Mb-72 Mb | 123 Mb-124 Mb | 31 Mb-32 Mb | 142 Mb-143 Mb | 125 Mb-126 Mb |
| chr1: | chr12: | chr19: | chr3: | chr6: |
| 72 Mb-73 Mb | 124 Mb-125 Mb | 32 Mb-33 Mb | 143 Mb-144 Mb | 126 Mb-127 Mb |
| chr1: | chr12: | chr19: | chr3: | chr6: |
| 73 Mb-74 Mb | 125 Mb-126 Mb | 33 Mb-34 Mb | 144 Mb-145 Mb | 127 Mb-128 Mb |
| chr1: | chr12: | chr19: | chr3: | chr6: |
| 74 Mb-75 Mb | 126 Mb-127 Mb | 34 Mb-35 Mb | 145 Mb-146 Mb | 128 Mb-129 Mb |
| chr1: | chr12: | chr19: | chr3: | chr6: |
| 75 Mb-76 Mb | 127 Mb-128 Mb | 35 Mb-36 Mb | 146 Mb-147 Mb | 129 Mb-130 Mb |
| chr1: | chr12: | chr19: | chr3: | chr6: |
| 76 Mb-77 Mb | 128 Mb-129 Mb | 36 Mb-37 Mb | 147 Mb-148 Mb | 130 Mb-131 Mb |
| chr1: | chr12: | chr19: | chr3: | chr6: |
| 77 Mb-78 Mb | 129 Mb-130 Mb | 37 Mb-38 Mb | 148 Mb-149 Mb | 131 Mb-132 Mb |
| chr1: | chr12: | chr19: | chr3: | chr6: |
| 78 Mb-79 Mb | 130 Mb-131 Mb | 38 Mb-39 Mb | 149 Mb-150 Mb | 132 Mb-133 Mb |
| chr1: | chr12: | chr19: | chr3: | chr6: |
| 79 Mb-8 Mb0 | 131 Mb-132 Mb | 39 Mb-40 Mb | 150 Mb-151 Mb | 133 Mb-134 Mb |
| chr1: | chr12: | chr19: | chr3: | chr6: |
| 8 Mb0-81 Mb | 132 Mb-133 Mb | 40 Mb-41 Mb | 151 Mb-152 Mb | 134 Mb-135 Mb |
| chr1: | chr12: | chr19: | chr3: | chr6: |
| 81 Mb-82 Mb | 133 Mb-133851895 | 41 Mb-42 Mb | 152 Mb-153 Mb | 135 Mb-136 Mb |
| chr1: | chr13: | chr19: | chr3: | chr6: |
| 82 Mb-83 Mb | 19 Mb-20 Mb | 42 Mb-43 Mb | 153 Mb-154 Mb | 136 Mb-137 Mb |
| chr1: | chr13: | chr19: | chr3: | chr6: |
| 83 Mb-84 Mb | 20 Mb-21 Mb | 43 Mb-44 Mb | 154 Mb-155 Mb | 137 Mb-138 Mb |
| chr1: | chr13: | chr19: | chr3: | chr6: |
| 84 Mb-85 Mb | 21 Mb-22 Mb | 44 Mb-45 Mb | 155 Mb-156 Mb | 138 Mb-139 Mb |
| chr1: | chr13: | chr19: | chr3: | chr6: |
| 85 Mb-86 Mb | 22 Mb-23 Mb | 45 Mb-46 Mb | 156 Mb-157 Mb | 139 Mb-140 Mb |
| chr1: | chr13: | chr19: | chr3: | chr6: |
| 86 Mb-87 Mb | 23 Mb-24 Mb | 46 Mb-47 Mb | 157 Mb-158 Mb | 140 Mb-141 Mb |
| chr1: | chr13: | chr19: | chr3: | chr6: |
| 87 Mb-88 Mb | 24 Mb-25 Mb | 47 Mb-48 Mb | 158 Mb-159 Mb | 141 Mb-142 Mb |
| chr1: | chr13: | chr19: | chr3: | chr6: |
| 88 Mb-89 Mb | 25 Mb-26 Mb | 48 Mb-49 Mb | 159 Mb-160 Mb | 142 Mb-143 Mb |
| chr1: | chr13: | chr19: | chr3: | chr6: |
| 89 Mb-90 Mb | 26 Mb-27 Mb | 49 Mb-50 Mb | 160 Mb-161 Mb | 143 Mb-144 Mb |
| chr1: | chr13: | chr19: | chr3: | chr6: |
| 90 Mb-91 Mb | 27 Mb-28 Mb | 50 Mb-51 Mb | 161 Mb-162 Mb | 144 Mb-145 Mb |
| chr1: | chr13: | chr19: | chr3: | chr6: |
| 91 Mb-92 Mb | 28 Mb-29 Mb | 51 Mb-52 Mb | 162 Mb-163 Mb | 145 Mb-146 Mb |
| chr1: | chr13: | chr19: | chr3: | chr6: |
| 92 Mb-93 Mb | 29 Mb-30 Mb | 52 Mb-53 Mb | 163 Mb-164 Mb | 146 Mb-147 Mb |
| chr1: | chr13: | chr19: | chr3: | chr6: |
| 93 Mb-94 Mb | 30 Mb-31 Mb | 53 Mb-54 Mb | 164 Mb-165 Mb | 147 Mb-148 Mb |
| chr1: | chr13: | chr19: | chr3: | chr6: |
| 94 Mb-95 Mb | 31 Mb-32 Mb | 54 Mb-55 Mb | 165 Mb-166 Mb | 148 Mb-149 Mb |
| chr1: | chr13: | chr19: | chr3: | chr6: |
| 95 Mb-96 Mb | 32 Mb-33 Mb | 55 Mb-56 Mb | 166 Mb-167 Mb | 149 Mb-150 Mb |
| chr1: | chr13: | chr19: | chr3: | chr6: |
| 96 Mb-97 Mb | 33 Mb-34 Mb | 56 Mb-57 Mb | 167 Mb-168 Mb | 150 Mb-151 Mb |
| chr1: | chr13: | chr19: | chr3: | chr6: |
| 97 Mb-98 Mb | 34 Mb-35 Mb | 57 Mb-58 Mb | 168 Mb-169 Mb | 151 Mb-152 Mb |
| chr1: | chr13: | chr19: | chr3: | chr6: |
| 98 Mb-99 Mb | 35 Mb-36 Mb | 58 Mb-59 Mb | 169 Mb-170 Mb | 152 Mb-153 Mb |
| chr1: | chr13: | chr19: | chr3: | chr6: |
| 99 Mb-100 Mb | 36 Mb-37 Mb | 59 Mb-59128983 | 170 Mb-171 Mb | 153 Mb-154 Mb |
| chr1: | chr13: | chr2: | chr3: | chr6: |
| 100 Mb-101 Mb | 37 Mb-38 Mb | 0-1 Mb | 171 Mb-172 Mb | 154 Mb-155 Mb |
| chr1: | chr13: | chr2: | chr3: | chr6: |
| 101 Mb-102 Mb | 38 Mb-39 Mb | 1 Mb-2 Mb | 172 Mb-173 Mb | 155 Mb-156 Mb |
| chr1: | chr13: | chr2: | chr3: | chr6: |
| 102 Mb-103 Mb | 39 Mb-40 Mb | 2 Mb-3 Mb | 173 Mb-174 Mb | 156 Mb-157 Mb |
| chr1: | chr13: | chr2: | chr3: | chr6: |
| 103 Mb-104 Mb | 40 Mb-41 Mb | 3 Mb-4 Mb | 174 Mb-175 Mb | 157 Mb-158 Mb |
| chr1: | chr13: | chr2: | chr3: | chr6: |
| 104 Mb-105 Mb | 41 Mb-42 Mb | 4 Mb-5 Mb | 175 Mb-176 Mb | 158 Mb-159 Mb |
| chr1: | chr13: | chr2: | chr3: | chr6: |
| 105 Mb-106 Mb | 42 Mb-43 Mb | 5 Mb-6 Mb | 176 Mb-177 Mb | 159 Mb-160 Mb |
| chr1: | chr13: | chr2: | chr3: | chr6: |
| 106 Mb-107 Mb | 43 Mb-44 Mb | 6 Mb-7 Mb | 177 Mb-178 Mb | 160 Mb-161 Mb |
| chr1: | chr13: | chr2: | chr3: | chr6: |
| 107 Mb-108 Mb | 44 Mb-45 Mb | 7 Mb-8 Mb | 178 Mb-179 Mb | 161 Mb-162 Mb |
| chr1: | chr13: | chr2: | chr3: | chr6: |
| 108 Mb-109 Mb | 45 Mb-46 Mb | 8 Mb-9 Mb | 179 Mb-18 Mb0 | 162 Mb-163 Mb |
| chr1: | chr13: | chr2: | chr3: | chr6: |
| 109 Mb-110 Mb | 46 Mb-47 Mb | 9 Mb-10 Mb | 18 Mb0-181 Mb | 163 Mb-164 Mb |
| chr1: | chr13: | chr2: | chr3: | chr6: |
| 110 Mb-111 Mb | 47 Mb-48 Mb | 10 Mb-11 Mb | 181 Mb-182 Mb | 164 Mb-165 Mb |
| chr1: | chr13: | chr2: | chr3: | chr6: |
| 111 Mb-112 Mb | 48 Mb-49 Mb | 11 Mb-12 Mb | 182 Mb-183 Mb | 165 Mb-166 Mb |
| chr1: | chr13: | chr2: | chr3: | chr6: |
| 112 Mb-113 Mb | 49 Mb-50 Mb | 12 Mb-13 Mb | 183 Mb-184 Mb | 166 Mb-167 Mb |
| chr1: | chr13: | chr2: | chr3: | chr6: |
| 113 Mb-114 Mb | 50 Mb-51 Mb | 13 Mb-14 Mb | 184 Mb-185 Mb | 167 Mb-168 Mb |
| chr1: | chr13: | chr2: | chr3: | chr6: |
| 114 Mb-115 Mb | 51 Mb-52 Mb | 14 Mb-15 Mb | 185 Mb-186 Mb | 168 Mb-169 Mb |
| chr1: | chr13: | chr2: | chr3: | chr6: |
| 115 Mb-116 Mb | 52 Mb-53 Mb | 15 Mb-16 Mb | 186 Mb-187 Mb | 169 Mb-170 Mb |
| chr1: | chr13: | chr2: | chr3: | chr6: |
| 116 Mb-117 Mb | 53 Mb-54 Mb | 16 Mb-17 Mb | 187 Mb-188 Mb | 170 Mb-171 Mb |
| chr1: | chr13: | chr2: | chr3: | chr7: |
| 117 Mb-118 Mb | 54 Mb-55 Mb | 17 Mb-18 Mb | 188 Mb-189 Mb | 0-1 Mb |
| chr1: | chr13: | chr2: | chr3: | chr7: |
| 118 Mb-119 Mb | 55 Mb-56 Mb | 18 Mb-19 Mb | 189 Mb-190 Mb | 1 Mb-2 Mb |
| chr1: | chr13: | chr2: | chr3: | chr7: |
| 119 Mb-120 Mb | 56 Mb-57 Mb | 19 Mb-20 Mb | 190 Mb-191 Mb | 2 Mb-3 Mb |
| chr1: | chr13: | chr2: | chr3: | chr7: |
| 120 Mb-121 Mb | 57 Mb-58 Mb | 20 Mb-21 Mb | 191 Mb-192 Mb | 3 Mb-4 Mb |
| chr1: | chr13: | chr2: | chr3: | chr7: |
| 121 Mb-122 Mb | 58 Mb-59 Mb | 21 Mb-22 Mb | 192 Mb-193 Mb | 4 Mb-5 Mb |
| chr1: | chr13: | chr2: | chr3: | chr7: |
| 142 Mb-143 Mb | 59 Mb-60 Mb | 22 Mb-23 Mb | 193 Mb-194 Mb | 5 Mb-6 Mb |
| chr1: | chr13: | chr2: | chr3: | chr7: |
| 143 Mb-144 Mb | 60 Mb-61 Mb | 23 Mb-24 Mb | 194 Mb-195 Mb | 6 Mb-7 Mb |
| chr1: | chr13: | chr2: | chr3: | chr7: |
| 144 Mb-145 Mb | 61 Mb-62 Mb | 24 Mb-25 Mb | 195 Mb-196 Mb | 7 Mb-8 Mb |
| chr1: | chr13: | chr2: | chr3: | chr7: |
| 145 Mb-146 Mb | 62 Mb-63 Mb | 25 Mb-26 Mb | 196 Mb-197 Mb | 8 Mb-9 Mb |
| chr1: | chr13: | chr2: | chr3: | chr7: |
| 146 Mb-147 Mb | 63 Mb-64 Mb | 26 Mb-27 Mb | 197 Mb-198 Mb | 9 Mb-10 Mb |
| chr1: | chr13: | chr2: | chr4: | chr7: |
| 147 Mb-148 Mb | 64 Mb-65 Mb | 27 Mb-28 Mb | 0-1 Mb | 10 Mb-11 Mb |
| chr1: | chr13: | chr2: | chr4: | chr7: |
| 148 Mb-149 Mb | 65 Mb-66 Mb | 28 Mb-29 Mb | 1 Mb-2 Mb | 11 Mb-12 Mb |
| chr1: | chr13: | chr2: | chr4: | chr7: |
| 149 Mb-150 Mb | 66 Mb-67 Mb | 29 Mb-30 Mb | 2 Mb-3 Mb | 12 Mb-13 Mb |
| chr1: | chr13: | chr2: | chr4: | chr7: |
| 150 Mb-151 Mb | 67 Mb-68 Mb | 30 Mb-31 Mb | 3 Mb-4 Mb | 13 Mb-14 Mb |
| chr1: | chr13: | chr2: | chr4: | chr7: |
| 151 Mb-152 Mb | 68 Mb-69 Mb | 31 Mb-32 Mb | 4 Mb-5 Mb | 14 Mb-15 Mb |
| chr1: | chr13: | chr2: | chr4: | chr7: |
| 152 Mb-153 Mb | 69 Mb-70 Mb | 32 Mb-33 Mb | 5 Mb-6 Mb | 15 Mb-16 Mb |
| chr1: | chr13: | chr2: | chr4: | chr7: |
| 153 Mb-154 Mb | 70 Mb-71 Mb | 33 Mb-34 Mb | 6 Mb-7 Mb | 16 Mb-17 Mb |
| chr1: | chr13: | chr2: | chr4: | chr7: |
| 154 Mb-155 Mb | 71 Mb-72 Mb | 34 Mb-35 Mb | 7 Mb-8 Mb | 17 Mb-18 Mb |
| chr1: | chr13: | chr2: | chr4: | chr7: |
| 155 Mb-156 Mb | 72 Mb-73 Mb | 35 Mb-36 Mb | 8 Mb-9 Mb | 18 Mb-19 Mb |
| chr1: | chr13: | chr2: | chr4: | chr7: |
| 156 Mb-157 Mb | 73 Mb-74 Mb | 36 Mb-37 Mb | 9 Mb-10 Mb | 19 Mb-20 Mb |
| chr1: | chr13: | chr2: | chr4: | chr7: |
| 157 Mb-158 Mb | 74 Mb-75 Mb | 37 Mb-38 Mb | 10 Mb-11 Mb | 20 Mb-21 Mb |
| chr1: | chr13: | chr2: | chr4: | chr7: |
| 158 Mb-159 Mb | 75 Mb-76 Mb | 38 Mb-39 Mb | 11 Mb-12 Mb | 21 Mb-22 Mb |
| chr1: | chr13: | chr2: | chr4: | chr7: |
| 159 Mb-160 Mb | 76 Mb-77 Mb | 39 Mb-40 Mb | 12 Mb-13 Mb | 22 Mb-23 Mb |
| chr1: | chr13: | chr2: | chr4: | chr7: |
| 160 Mb-161 Mb | 77 Mb-78 Mb | 40 Mb-41 Mb | 13 Mb-14 Mb | 23 Mb-24 Mb |
| chr1: | chr13: | chr2: | chr4: | chr7: |
| 161 Mb-162 Mb | 78 Mb-79 Mb | 41 Mb-42 Mb | 14 Mb-15 Mb | 24 Mb-25 Mb |
| chr1: | chr13: | chr2: | chr4: | chr7: |
| 162 Mb-163 Mb | 79 Mb-8 Mb0 | 42 Mb-43 Mb | 15 Mb-16 Mb | 25 Mb-26 Mb |
| chr1: | chr13: | chr2: | chr4: | chr7: |
| 163 Mb-164 Mb | 8 Mb0-81 Mb | 43 Mb-44 Mb | 16 Mb-17 Mb | 26 Mb-27 Mb |
| chr1: | chr13: | chr2: | chr4: | chr7: |
| 164 Mb-165 Mb | 81 Mb-82 Mb | 44 Mb-45 Mb | 17 Mb-18 Mb | 27 Mb-28 Mb |
| chr1: | chr13: | chr2: | chr4: | chr7: |
| 165 Mb-166 Mb | 82 Mb-83 Mb | 45 Mb-46 Mb | 18 Mb-19 Mb | 28 Mb-29 Mb |
| chr1: | chr13: | chr2: | chr4: | chr7: |
| 166 Mb-167 Mb | 83 Mb-84 Mb | 46 Mb-47 Mb | 19 Mb-20 Mb | 29 Mb-30 Mb |
| chr1: | chr13: | chr2: | chr4: | chr7: |
| 167 Mb-168 Mb | 84 Mb-85 Mb | 47 Mb-48 Mb | 20 Mb-21 Mb | 30 Mb-31 Mb |
| chr1: | chr13: | chr2: | chr4: | chr7: |
| 168 Mb-169 Mb | 85 Mb-86 Mb | 48 Mb-49 Mb | 21 Mb-22 Mb | 31 Mb-32 Mb |
| chr1: | chr13: | chr2: | chr4: | chr7: |
| 169 Mb-170 Mb | 86 Mb-87 Mb | 49 Mb-50 Mb | 22 Mb-23 Mb | 32 Mb-33 Mb |
| chr1: | chr13: | chr2: | chr4: | chr7: |
| 170 Mb-171 Mb | 87 Mb-88 Mb | 50 Mb-51 Mb | 23 Mb-24 Mb | 33 Mb-34 Mb |
| chr1: | chr13: | chr2: | chr4: | chr7: |
| 171 Mb-172 Mb | 88 Mb-89 Mb | 51 Mb-52 Mb | 24 Mb-25 Mb | 34 Mb-35 Mb |
| chr1: | chr13: | chr2: | chr4: | chr7: |
| 172 Mb-173 Mb | 89 Mb-90 Mb | 52 Mb-53 Mb | 25 Mb-26 Mb | 35 Mb-36 Mb |
| chr1: | chr13: | chr2: | chr4: | chr7: |
| 173 Mb-174 Mb | 90 Mb-91 Mb | 53 Mb-54 Mb | 26 Mb-27 Mb | 36 Mb-37 Mb |
| chr1: | chr13: | chr2: | chr4: | chr7: |
| 174 Mb-175 Mb | 91 Mb-92 Mb | 54 Mb-55 Mb | 27 Mb-28 Mb | 37 Mb-38 Mb |
| chr1: | chr13: | chr2: | chr4: | chr7: |
| 175 Mb-176 Mb | 92 Mb-93 Mb | 55 Mb-56 Mb | 28 Mb-29 Mb | 38 Mb-39 Mb |
| chr1: | chr13: | chr2: | chr4: | chr7: |
| 176 Mb-177 Mb | 93 Mb-94 Mb | 56 Mb-57 Mb | 29 Mb-30 Mb | 39 Mb-40 Mb |
| chr1: | chr13: | chr2: | chr4: | chr7: |
| 177 Mb-178 Mb | 94 Mb-95 Mb | 57 Mb-58 Mb | 30 Mb-31 Mb | 40 Mb-41 Mb |
| chr1: | chr13: | chr2: | chr4: | chr7: |
| 178 Mb-179 Mb | 95 Mb-96 Mb | 58 Mb-59 Mb | 31 Mb-32 Mb | 41 Mb-42 Mb |
| chr1: | chr13: | chr2: | chr4: | chr7: |
| 179 Mb-18 Mb0 | 96 Mb-97 Mb | 59 Mb-60 Mb | 32 Mb-33 Mb | 42 Mb-43 Mb |
| chr1: | chr13: | chr2: | chr4: | chr7: |
| 18 Mb0-181 Mb | 97 Mb-98 Mb | 60 Mb-61 Mb | 33 Mb-34 Mb | 43 Mb-44 Mb |
| chr1: | chr13: | chr2: | chr4: | chr7: |
| 181 Mb-182 Mb | 98 Mb-99 Mb | 61 Mb-62 Mb | 34 Mb-35 Mb | 44 Mb-45 Mb |
| chr1: | chr13: | chr2: | chr4: | chr7: |
| 182 Mb-183 Mb | 99 Mb-100 Mb | 62 Mb-63 Mb | 35 Mb-36 Mb | 45 Mb-46 Mb |
| chr1: | chr13: | chr2: | chr4: | chr7: |
| 183 Mb-184 Mb | 100 Mb-101 Mb | 63 Mb-64 Mb | 36 Mb-37 Mb | 46 Mb-47 Mb |
| chr1: | chr13: | chr2: | chr4: | chr7: |
| 184 Mb-185 Mb | 101 Mb-102 Mb | 64 Mb-65 Mb | 37 Mb-38 Mb | 47 Mb-48 Mb |
| chr1: | chr13: | chr2: | chr4: | chr7: |
| 185 Mb-186 Mb | 102 Mb-103 Mb | 65 Mb-66 Mb | 38 Mb-39 Mb | 48 Mb-49 Mb |
| chr1: | chr13: | chr2: | chr4: | chr7: |
| 186 Mb-187 Mb | 103 Mb-104 Mb | 66 Mb-67 Mb | 39 Mb-40 Mb | 49 Mb-50 Mb |
| chr1: | chr13: | chr2: | chr4: | chr7: |
| 187 Mb-188 Mb | 104 Mb-105 Mb | 67 Mb-68 Mb | 40 Mb-41 Mb | 50 Mb-51 Mb |
| chr1: | chr13: | chr2: | chr4: | chr7: |
| 188 Mb-189 Mb | 105 Mb-106 Mb | 68 Mb-69 Mb | 41 Mb-42 Mb | 51 Mb-52 Mb |
| chr1: | chr13: | chr2: | chr4: | chr7: |
| 189 Mb-190 Mb | 106 Mb-107 Mb | 69 Mb-70 Mb | 42 Mb-43 Mb | 52 Mb-53 Mb |
| chr1: | chr13: | chr2: | chr4: | chr7: |
| 190 Mb-191 Mb | 107 Mb-108 Mb | 70 Mb-71 Mb | 43 Mb-44 Mb | 53 Mb-54 Mb |
| chr1: | chr13: | chr2: | chr4: | chr7: |
| 191 Mb-192 Mb | 108 Mb-109 Mb | 71 Mb-72 Mb | 44 Mb-45 Mb | 54 Mb-55 Mb |
| chr1: | chr13: | chr2: | chr4: | chr7: |
| 192 Mb-193 Mb | 109 Mb-110 Mb | 72 Mb-73 Mb | 45 Mb-46 Mb | 55 Mb-56 Mb |
| chr1: | chr13: | chr2: | chr4: | chr7: |
| 193 Mb-194 Mb | 110 Mb-111 Mb | 73 Mb-74 Mb | 46 Mb-47 Mb | 56 Mb-57 Mb |
| chr1: | chr13: | chr2: | chr4: | chr7: |
| 194 Mb-195 Mb | 111 Mb-112 Mb | 74 Mb-75 Mb | 47 Mb-48 Mb | 57 Mb-58 Mb |
| chr1: | chr13: | chr2: | chr4: | chr7: |
| 195 Mb-196 Mb | 112 Mb-113 Mb | 75 Mb-76 Mb | 48 Mb-49 Mb | 62 Mb-63 Mb |
| chr1: | chr13: | chr2: | chr4: | chr7: |
| 196 Mb-197 Mb | 113 Mb-114 Mb | 76 Mb-77 Mb | 49 Mb-50 Mb | 63 Mb-64 Mb |
| chr1: | chr13: | chr2: | chr4: | chr7: |
| 197 Mb-198 Mb | 114 Mb-115 Mb | 77 Mb-78 Mb | 52 Mb-53 Mb | 64 Mb-65 Mb |
| chr1: | chr13: | chr2: | chr4: | chr7: |
| 198 Mb-199 Mb | 115 Mb-115169878 | 78 Mb-79 Mb | 53 Mb-54 Mb | 65 Mb-66 Mb |
| chr1: | chr14: | chr2: | chr4: | chr7: |
| 199 Mb-20 Mb0 | 19 Mb-20 Mb | 79 Mb-8 Mb0 | 54 Mb-55 Mb | 66 Mb-67 Mb |
| chr1: | chr14: | chr2: | chr4: | chr7: |
| 20 Mb0-201 Mb | 20 Mb-21 Mb | 8 Mb0-81 Mb | 55 Mb-56 Mb | 67 Mb-68 Mb |
| chr1: | chr14: | chr2: | chr4: | chr7: |
| 201 Mb-202 Mb | 21 Mb-22 Mb | 81 Mb-82 Mb | 56 Mb-57 Mb | 68 Mb-69 Mb |
| chr1: | chr14: | chr2: | chr4: | chr7: |
| 202 Mb-203 Mb | 22 Mb-23 Mb | 82 Mb-83 Mb | 57 Mb-58 Mb | 69 Mb-70 Mb |
| chr1: | chr14: | chr2: | chr4: | chr7: |
| 203 Mb-204 Mb | 23 Mb-24 Mb | 83 Mb-84 Mb | 58 Mb-59 Mb | 70 Mb-71 Mb |
| chr1: | chr14: | chr2: | chr4: | chr7: |
| 204 Mb-205 Mb | 24 Mb-25 Mb | 84 Mb-85 Mb | 59 Mb-60 Mb | 71 Mb-72 Mb |
| chr1: | chr14: | chr2: | chr4: | chr7: |
| 205 Mb-206 Mb | 25 Mb-26 Mb | 85 Mb-86 Mb | 60 Mb-61 Mb | 72 Mb-73 Mb |
| chr1: | chr14: | chr2: | chr4: | chr7: |
| 206 Mb-207 Mb | 26 Mb-27 Mb | 86 Mb-87 Mb | 61 Mb-62 Mb | 73 Mb-74 Mb |
| chr1: | chr14: | chr2: | chr4: | chr7: |
| 207 Mb-208 Mb | 27 Mb-28 Mb | 87 Mb-88 Mb | 62 Mb-63 Mb | 74 Mb-75 Mb |
| chr1: | chr14: | chr2: | chr4: | chr7: |
| 208 Mb-209 Mb | 28 Mb-29 Mb | 88 Mb-89 Mb | 63 Mb-64 Mb | 75 Mb-76 Mb |
| chr1: | chr14: | chr2: | chr4: | chr7: |
| 209 Mb-210 Mb | 29 Mb-30 Mb | 89 Mb-90 Mb | 64 Mb-65 Mb | 76 Mb-77 Mb |
| chr1: | chr14: | chr2: | chr4: | chr7: |
| 210 Mb-211 Mb | 30 Mb-31 Mb | 90 Mb-91 Mb | 65 Mb-66 Mb | 77 Mb-78 Mb |
| chr1: | chr14: | chr2: | chr4: | chr7: |
| 211 Mb-212 Mb | 31 Mb-32 Mb | 91 Mb-92 Mb | 66 Mb-67 Mb | 78 Mb-79 Mb |
| chr1: | chr14: | chr2: | chr4: | chr7: |
| 212 Mb-213 Mb | 32 Mb-33 Mb | 92 Mb-93 Mb | 67 Mb-68 Mb | 79 Mb-8 Mb0 |
| chr1: | chr14: | chr2: | chr4: | chr7: |
| 213 Mb-214 Mb | 33 Mb-34 Mb | 95 Mb-96 Mb | 68 Mb-69 Mb | 8 Mb0-81 Mb |
| chr1: | chr14: | chr2: | chr4: | chr7: |
| 214 Mb-215 Mb | 34 Mb-35 Mb | 96 Mb-97 Mb | 69 Mb-70 Mb | 81 Mb-82 Mb |
| chr1: | chr14: | chr2: | chr4: | chr7: |
| 215 Mb-216 Mb | 35 Mb-36 Mb | 97 Mb-98 Mb | 70 Mb-71 Mb | 82 Mb-83 Mb |
| chr1: | chr14: | chr2: | chr4: | chr7: |
| 216 Mb-217 Mb | 36 Mb-37 Mb | 98 Mb-99 Mb | 71 Mb-72 Mb | 83 Mb-84 Mb |
| chr1: | chr14: | chr2: | chr4: | chr7: |
| 217 Mb-218 Mb | 37 Mb-38 Mb | 99 Mb-100 Mb | 72 Mb-73 Mb | 84 Mb-85 Mb |
| chr1: | chr14: | chr2: | chr4: | chr7: |
| 218 Mb-219 Mb | 38 Mb-39 Mb | 100 Mb-101 Mb | 73 Mb-74 Mb | 85 Mb-86 Mb |
| chr1: | chr14: | chr2: | chr4: | chr7: |
| 219 Mb-220 Mb | 39 Mb-40 Mb | 101 Mb-102 Mb | 74 Mb-75 Mb | 86 Mb-87 Mb |
| chr1: | chr14: | chr2: | chr4: | chr7: |
| 220 Mb-221 Mb | 40 Mb-41 Mb | 102 Mb-103 Mb | 75 Mb-76 Mb | 87 Mb-88 Mb |
| chr1: | chr14: | chr2: | chr4: | chr7: |
| 221 Mb-222 Mb | 41 Mb-42 Mb | 103 Mb-104 Mb | 76 Mb-77 Mb | 88 Mb-89 Mb |
| chr1: | chr14: | chr2: | chr4: | chr7: |
| 222 Mb-223 Mb | 42 Mb-43 Mb | 104 Mb-105 Mb | 77 Mb-78 Mb | 89 Mb-90 Mb |
| chr1: | chr14: | chr2: | chr4: | chr7: |
| 223 Mb-224 Mb | 43 Mb-44 Mb | 105 Mb-106 Mb | 78 Mb-79 Mb | 90 Mb-91 Mb |
| chr1: | chr14: | chr2: | chr4: | chr7: |
| 224 Mb-225 Mb | 44 Mb-45 Mb | 106 Mb-107 Mb | 79 Mb-8 Mb0 | 91 Mb-92 Mb |
| chr1: | chr14: | chr2: | chr4: | chr7: |
| 225 Mb-226 Mb | 45 Mb-46 Mb | 107 Mb-108 Mb | 8 Mb0-81 Mb | 92 Mb-93 Mb |
| chr1: | chr14: | chr2: | chr4: | chr7: |
| 226 Mb-227 Mb | 46 Mb-47 Mb | 108 Mb-109 Mb | 81 Mb-82 Mb | 93 Mb-94 Mb |
| chr1: | chr14: | chr2: | chr4: | chr7: |
| 227 Mb-228 Mb | 47 Mb-48 Mb | 109 Mb-110 Mb | 82 Mb-83 Mb | 94 Mb-95 Mb |
| chr1: | chr14: | chr2: | chr4: | chr7: |
| 228 Mb-229 Mb | 48 Mb-49 Mb | 110 Mb-111 Mb | 83 Mb-84 Mb | 95 Mb-96 Mb |
| chr1: | chr14: | chr2: | chr4: | chr7: |
| 229 Mb-230 Mb | 49 Mb-50 Mb | 111 Mb-112 Mb | 84 Mb-85 Mb | 96 Mb-97 Mb |
| chr1: | chr14: | chr2: | chr4: | chr7: |
| 230 Mb-231 Mb | 50 Mb-51 Mb | 112 Mb-113 Mb | 85 Mb-86 Mb | 97 Mb-98 Mb |
| chr1: | chr14: | chr2: | chr4: | chr7: |
| 231 Mb-232 Mb | 51 Mb-52 Mb | 113 Mb-114 Mb | 86 Mb-87 Mb | 98 Mb-99 Mb |
| chr1: | chr14: | chr2: | chr4: | chr7: |
| 232 Mb-233 Mb | 52 Mb-53 Mb | 114 Mb-115 Mb | 87 Mb-88 Mb | 99 Mb-100 Mb |
| chr1: | chr14: | chr2: | chr4: | chr7: |
| 233 Mb-234 Mb | 53 Mb-54 Mb | 115 Mb-116 Mb | 88 Mb-89 Mb | 100 Mb-101 Mb |
| chr1: | chr14: | chr2: | chr4: | chr7: |
| 234 Mb-235 Mb | 54 Mb-55 Mb | 116 Mb-117 Mb | 89 Mb-90 Mb | 101 Mb-102 Mb |
| chr1: | chr14: | chr2: | chr4: | chr7: |
| 235 Mb-236 Mb | 55 Mb-56 Mb | 117 Mb-118 Mb | 90 Mb-91 Mb | 102 Mb-103 Mb |
| chr1: | chr14: | chr2: | chr4: | chr7: |
| 236 Mb-237 Mb | 56 Mb-57 Mb | 118 Mb-119 Mb | 91 Mb-92 Mb | 103 Mb-104 Mb |
| chr1: | chr14: | chr2: | chr4: | chr7: |
| 237 Mb-238 Mb | 57 Mb-58 Mb | 119 Mb-120 Mb | 92 Mb-93 Mb | 104 Mb-105 Mb |
| chr1: | chr14: | chr2: | chr4: | chr7: |
| 238 Mb-239 Mb | 58 Mb-59 Mb | 120 Mb-121 Mb | 93 Mb-94 Mb | 105 Mb-106 Mb |
| chr1: | chr14: | chr2: | chr4: | chr7: |
| 239 Mb-240 Mb | 59 Mb-60 Mb | 121 Mb-122 Mb | 94 Mb-95 Mb | 106 Mb-107 Mb |
| chr1: | chr14: | chr2: | chr4: | chr7: |
| 240 Mb-241 Mb | 60 Mb-61 Mb | 122 Mb-123 Mb | 95 Mb-96 Mb | 107 Mb-108 Mb |
| chr1: | chr14: | chr2: | chr4: | chr7: |
| 241 Mb-242 Mb | 61 Mb-62 Mb | 123 Mb-124 Mb | 96 Mb-97 Mb | 108 Mb-109 Mb |
| chr1: | chr14: | chr2: | chr4: | chr7: |
| 242 Mb-243 Mb | 62 Mb-63 Mb | 124 Mb-125 Mb | 97 Mb-98 Mb | 109 Mb-110 Mb |
| chr1: | chr14: | chr2: | chr4: | chr7: |
| 243 Mb-244 Mb | 63 Mb-64 Mb | 125 Mb-126 Mb | 98 Mb-99 Mb | 110 Mb-111 Mb |
| chr1: | chr14: | chr2: | chr4: | chr7: |
| 244 Mb-245 Mb | 64 Mb-65 Mb | 126 Mb-127 Mb | 99 Mb-100 Mb | 111 Mb-112 Mb |
| chr1: | chr14: | chr2: | chr4: | chr7: |
| 245 Mb-246 Mb | 65 Mb-66 Mb | 127 Mb-128 Mb | 100 Mb-101 Mb | 112 Mb-113 Mb |
| chr1: | chr14: | chr2: | chr4: | chr7: |
| 246 Mb-247 Mb | 66 Mb-67 Mb | 128 Mb-129 Mb | 101 Mb-102 Mb | 113 Mb-114 Mb |
| chr1: | chr14: | chr2: | chr4: | chr7: |
| 247 Mb-248 Mb | 67 Mb-68 Mb | 129 Mb-130 Mb | 102 Mb-103 Mb | 114 Mb-115 Mb |
| chr1: | chr14: | chr2: | chr4: | chr7: |
| 248 Mb-249 Mb | 68 Mb-69 Mb | 130 Mb-131 Mb | 103 Mb-104 Mb | 115 Mb-116 Mb |
| chr1: | chr14: | chr2: | chr4: | chr7: |
| 249 Mb-249250621 | 69 Mb-70 Mb | 131 Mb-132 Mb | 104 Mb-105 Mb | 116 Mb-117 Mb |
| chr10: | chr14: | chr2: | chr4: | chr7: |
| 0-1 Mb | 70 Mb-71 Mb | 132 Mb-133 Mb | 105 Mb-106 Mb | 117 Mb-118 Mb |
| chr10: | chr14: | chr2: | chr4: | chr7: |
| 1 Mb-2 Mb | 71 Mb-72 Mb | 133 Mb-134 Mb | 106 Mb-107 Mb | 118 Mb-119 Mb |
| chr10: | chr14: | chr2: | chr4: | chr7: |
| 2 Mb-3 Mb | 72 Mb-73 Mb | 134 Mb-135 Mb | 107 Mb-108 Mb | 119 Mb-120 Mb |
| chr10: | chr14: | chr2: | chr4: | chr7: |
| 3 Mb-4 Mb | 73 Mb-74 Mb | 135 Mb-136 Mb | 108 Mb-109 Mb | 120 Mb-121 Mb |
| chr10: | chr14: | chr2: | chr4: | chr7: |
| 4 Mb-5 Mb | 74 Mb-75 Mb | 136 Mb-137 Mb | 109 Mb-110 Mb | 121 Mb-122 Mb |
| chr10: | chr14: | chr2: | chr4: | chr7: |
| 5 Mb-6 Mb | 75 Mb-76 Mb | 137 Mb-138 Mb | 110 Mb-111 Mb | 122 Mb-123 Mb |
| chr10: | chr14: | chr2: | chr4: | chr7: |
| 6 Mb-7 Mb | 76 Mb-77 Mb | 138 Mb-139 Mb | 111 Mb-112 Mb | 123 Mb-124 Mb |
| chr10: | chr14: | chr2: | chr4: | chr7: |
| 7 Mb-8 Mb | 77 Mb-78 Mb | 139 Mb-140 Mb | 112 Mb-113 Mb | 124 Mb-125 Mb |
| chr10: | chr14: | chr2: | chr4: | chr7: |
| 8 Mb-9 Mb | 78 Mb-79 Mb | 140 Mb-141 Mb | 113 Mb-114 Mb | 125 Mb-126 Mb |
| chr10: | chr14: | chr2: | chr4: | chr7: |
| 9 Mb-10 Mb | 79 Mb-8 Mb0 | 141 Mb-142 Mb | 114 Mb-115 Mb | 126 Mb-127 Mb |
| chr10: | chr14: | chr2: | chr4: | chr7: |
| 10 Mb-11 Mb | 8 Mb0-81 Mb | 142 Mb-143 Mb | 115 Mb-116 Mb | 127 Mb-128 Mb |
| chr10: | chr14: | chr2: | chr4: | chr7: |
| 11 Mb-12 Mb | 81 Mb-82 Mb | 143 Mb-144 Mb | 116 Mb-117 Mb | 128 Mb-129 Mb |
| chr10: | chr14: | chr2: | chr4: | chr7: |
| 12 Mb-13 Mb | 82 Mb-83 Mb | 144 Mb-145 Mb | 117 Mb-118 Mb | 129 Mb-130 Mb |
| chr10: | chr14: | chr2: | chr4: | chr7: |
| 13 Mb-14 Mb | 83 Mb-84 Mb | 145 Mb-146 Mb | 118 Mb-119 Mb | 130 Mb-131 Mb |
| chr10: | chr14: | chr2: | chr4: | chr7: |
| 14 Mb-15 Mb | 84 Mb-85 Mb | 146 Mb-147 Mb | 119 Mb-120 Mb | 131 Mb-132 Mb |
| chr10: | chr14: | chr2: | chr4: | chr7: |
| 15 Mb-16 Mb | 85 Mb-86 Mb | 147 Mb-148 Mb | 120 Mb-121 Mb | 132 Mb-133 Mb |
| chr10: | chr14: | chr2: | chr4: | chr7: |
| 16 Mb-17 Mb | 86 Mb-87 Mb | 148 Mb-149 Mb | 121 Mb-122 Mb | 133 Mb-134 Mb |
| chr10: | chr14: | chr2: | chr4: | chr7: |
| 17 Mb-18 Mb | 87 Mb-88 Mb | 149 Mb-150 Mb | 122 Mb-123 Mb | 134 Mb-135 Mb |
| chr10: | chr14: | chr2: | chr4: | chr7: |
| 18 Mb-19 Mb | 88 Mb-89 Mb | 150 Mb-151 Mb | 123 Mb-124 Mb | 135 Mb-136 Mb |
| chr10: | chr14: | chr2: | chr4: | chr7: |
| 19 Mb-20 Mb | 89 Mb-90 Mb | 151 Mb-152 Mb | 124 Mb-125 Mb | 136 Mb-137 Mb |
| chr10: | chr14: | chr2: | chr4: | chr7: |
| 20 Mb-21 Mb | 90 Mb-91 Mb | 152 Mb-153 Mb | 125 Mb-126 Mb | 137 Mb-138 Mb |
| chr10: | chr14: | chr2: | chr4: | chr7: |
| 21 Mb-22 Mb | 91 Mb-92 Mb | 153 Mb-154 Mb | 126 Mb-127 Mb | 138 Mb-139 Mb |
| chr10: | chr14: | chr2: | chr4: | chr7: |
| 22 Mb-23 Mb | 92 Mb-93 Mb | 154 Mb-155 Mb | 127 Mb-128 Mb | 139 Mb-140 Mb |
| chr10: | chr14: | chr2: | chr4: | chr7: |
| 23 Mb-24 Mb | 93 Mb-94 Mb | 155 Mb-156 Mb | 128 Mb-129 Mb | 140 Mb-141 Mb |
| chr10: | chr14: | chr2: | chr4: | chr7: |
| 24 Mb-25 Mb | 94 Mb-95 Mb | 156 Mb-157 Mb | 129 Mb-130 Mb | 141 Mb-142 Mb |
| chr10: | chr14: | chr2: | chr4: | chr7: |
| 25 Mb-26 Mb | 95 Mb-96 Mb | 157 Mb-158 Mb | 130 Mb-131 Mb | 142 Mb-143 Mb |
| chr10: | chr14: | chr2: | chr4: | chr7: |
| 26 Mb-27 Mb | 96 Mb-97 Mb | 158 Mb-159 Mb | 131 Mb-132 Mb | 143 Mb-144 Mb |
| chr10: | chr14: | chr2: | chr4: | chr7: |
| 27 Mb-28 Mb | 97 Mb-98 Mb | 159 Mb-160 Mb | 132 Mb-133 Mb | 144 Mb-145 Mb |
| chr10: | chr14: | chr2: | chr4: | chr7: |
| 28 Mb-29 Mb | 98 Mb-99 Mb | 160 Mb-161 Mb | 133 Mb-134 Mb | 145 Mb-146 Mb |
| chr10: | chr14: | chr2: | chr4: | chr7: |
| 29 Mb-30 Mb | 99 Mb-100 Mb | 161 Mb-162 Mb | 134 Mb-135 Mb | 146 Mb-147 Mb |
| chr10: | chr14: | chr2: | chr4: | chr7: |
| 30 Mb-31 Mb | 100 Mb-101 Mb | 162 Mb-163 Mb | 135 Mb-136 Mb | 147 Mb-148 Mb |
| chr10: | chr14: | chr2: | chr4: | chr7: |
| 31 Mb-32 Mb | 101 Mb-102 Mb | 163 Mb-164 Mb | 136 Mb-137 Mb | 148 Mb-149 Mb |
| chr10: | chr14: | chr2: | chr4: | chr7: |
| 32 Mb-33 Mb | 102 Mb-103 Mb | 164 Mb-165 Mb | 137 Mb-138 Mb | 149 Mb-150 Mb |
| chr10: | chr14: | chr2: | chr4: | chr7: |
| 33 Mb-34 Mb | 103 Mb-104 Mb | 165 Mb-166 Mb | 138 Mb-139 Mb | 150 Mb-151 Mb |
| chr10: | chr14: | chr2: | chr4: | chr7: |
| 34 Mb-35 Mb | 104 Mb-105 Mb | 166 Mb-167 Mb | 139 Mb-140 Mb | 151 Mb-152 Mb |
| chr10: | chr14: | chr2: | chr4: | chr7: |
| 35 Mb-36 Mb | 105 Mb-106 Mb | 167 Mb-168 Mb | 140 Mb-141 Mb | 152 Mb-153 Mb |
| chr10: | chr14: | chr2: | chr4: | chr7: |
| 36 Mb-37 Mb | 106 Mb-107 Mb | 168 Mb-169 Mb | 141 Mb-142 Mb | 153 Mb-154 Mb |
| chr10: | chr14: | chr2: | chr4: | chr7: |
| 37 Mb-38 Mb | 107 Mb-107349540 | 169 Mb-170 Mb | 142 Mb-143 Mb | 154 Mb-155 Mb |
| chr10: | chr15: | chr2: | chr4: | chr7: |
| 38 Mb-39 Mb | 20 Mb-21 Mb | 170 Mb-171 Mb | 143 Mb-144 Mb | 155 Mb-156 Mb |
| chr10: | chr15: | chr2: | chr4: | chr7: |
| 39 Mb-40 Mb | 21 Mb-22 Mb | 171 Mb-172 Mb | 144 Mb-145 Mb | 156 Mb-157 Mb |
| chr10: | chr15: | chr2: | chr4: | chr7: |
| 42 Mb-43 Mb | 22 Mb-23 Mb | 172 Mb-173 Mb | 145 Mb-146 Mb | 157 Mb-158 Mb |
| chr10: | chr15: | chr2: | chr4: | chr7: |
| 43 Mb-44 Mb | 23 Mb-24 Mb | 173 Mb-174 Mb | 146 Mb-147 Mb | 158 Mb-159 Mb |
| chr10: | chr15: | chr2: | chr4: | chr7: |
| 44 Mb-45 Mb | 24 Mb-25 Mb | 174 Mb-175 Mb | 147 Mb-148 Mb | 159 Mb-159138663 |
| chr10: | chr15: | chr2: | chr4: | chr8: |
| 45 Mb-46 Mb | 25 Mb-26 Mb | 175 Mb-176 Mb | 148 Mb-149 Mb | 0-1 Mb |
| chr10: | chr15: | chr2: | chr4: | chr8: |
| 46 Mb-47 Mb | 26 Mb-27 Mb | 176 Mb-177 Mb | 149 Mb-150 Mb | 1 Mb-2 Mb |
| chr10: | chr15: | chr2: | chr4: | chr8: |
| 47 Mb-48 Mb | 27 Mb-28 Mb | 177 Mb-178 Mb | 150 Mb-151 Mb | 2 Mb-3 Mb |
| chr10: | chr15: | chr2: | chr4: | chr8: |
| 48 Mb-49 Mb | 28 Mb-29 Mb | 178 Mb-179 Mb | 151 Mb-152 Mb | 3 Mb-4 Mb |
| chr10: | chr15: | chr2: | chr4: | chr8: |
| 49 Mb-50 Mb | 29 Mb-30 Mb | 179 Mb-18 Mb0 | 152 Mb-153 Mb | 4 Mb-5 Mb |
| chr10: | chr15: | chr2: | chr4: | chr8: |
| 50 Mb-51 Mb | 30 Mb-31 Mb | 18 Mb0-181 Mb | 153 Mb-154 Mb | 5 Mb-6 Mb |
| chr10: | chr15: | chr2: | chr4: | chr8: |
| 51 Mb-52 Mb | 31 Mb-32 Mb | 181 Mb-182 Mb | 154 Mb-155 Mb | 6 Mb-7 Mb |
| chr10: | chr15: | chr2: | chr4: | chr8: |
| 52 Mb-53 Mb | 32 Mb-33 Mb | 182 Mb-183 Mb | 155 Mb-156 Mb | 7 Mb-8 Mb |
| chr10: | chr15: | chr2: | chr4: | chr8: |
| 53 Mb-54 Mb | 33 Mb-34 Mb | 183 Mb-184 Mb | 156 Mb-157 Mb | 8 Mb-9 Mb |
| chr10: | chr15: | chr2: | chr4: | chr8: |
| 54 Mb-55 Mb | 34 Mb-35 Mb | 184 Mb-185 Mb | 157 Mb-158 Mb | 9 Mb-10 Mb |
| chr10: | chr15: | chr2: | chr4: | chr8: |
| 55 Mb-56 Mb | 35 Mb-36 Mb | 185 Mb-186 Mb | 158 Mb-159 Mb | 10 Mb-11 Mb |
| chr10: | chr15: | chr2: | chr4: | chr8: |
| 56 Mb-57 Mb | 36 Mb-37 Mb | 186 Mb-187 Mb | 159 Mb-160 Mb | 11 Mb-12 Mb |
| chr10: | chr15: | chr2: | chr4: | chr8: |
| 57 Mb-58 Mb | 37 Mb-38 Mb | 187 Mb-188 Mb | 160 Mb-161 Mb | 12 Mb-13 Mb |
| chr10: | chr15: | chr2: | chr4: | chr8: |
| 58 Mb-59 Mb | 38 Mb-39 Mb | 188 Mb-189 Mb | 161 Mb-162 Mb | 13 Mb-14 Mb |
| chr10: | chr15: | chr2: | chr4: | chr8: |
| 59 Mb-60 Mb | 39 Mb-40 Mb | 189 Mb-190 Mb | 162 Mb-163 Mb | 14 Mb-15 Mb |
| chr10: | chr15: | chr2: | chr4: | chr8: |
| 60 Mb-61 Mb | 40 Mb-41 Mb | 190 Mb-191 Mb | 163 Mb-164 Mb | 15 Mb-16 Mb |
| chr10: | chr15: | chr2: | chr4: | chr8: |
| 61 Mb-62 Mb | 41 Mb-42 Mb | 191 Mb-192 Mb | 164 Mb-165 Mb | 16 Mb-17 Mb |
| chr10: | chr15: | chr2: | chr4: | chr8: |
| 62 Mb-63 Mb | 42 Mb-43 Mb | 192 Mb-193 Mb | 165 Mb-166 Mb | 17 Mb-18 Mb |
| chr10: | chr15: | chr2: | chr4: | chr8: |
| 63 Mb-64 Mb | 43 Mb-44 Mb | 193 Mb-194 Mb | 166 Mb-167 Mb | 18 Mb-19 Mb |
| chr10: | chr15: | chr2: | chr4: | chr8: |
| 64 Mb-65 Mb | 44 Mb-45 Mb | 194 Mb-195 Mb | 167 Mb-168 Mb | 19 Mb-20 Mb |
| chr10: | chr15: | chr2: | chr4: | chr8: |
| 65 Mb-66 Mb | 45 Mb-46 Mb | 195 Mb-196 Mb | 168 Mb-169 Mb | 20 Mb-21 Mb |
| chr10: | chr15: | chr2: | chr4: | chr8: |
| 66 Mb-67 Mb | 46 Mb-47 Mb | 196 Mb-197 Mb | 169 Mb-170 Mb | 21 Mb-22 Mb |
| chr10: | chr15: | chr2: | chr4: | chr8: |
| 67 Mb-68 Mb | 47 Mb-48 Mb | 197 Mb-198 Mb | 170 Mb-171 Mb | 22 Mb-23 Mb |
| chr10: | chr15: | chr2: | chr4: | chr8: |
| 68 Mb-69 Mb | 48 Mb-49 Mb | 198 Mb-199 Mb | 171 Mb-172 Mb | 23 Mb-24 Mb |
| chr10: | chr15: | chr2: | chr4: | chr8: |
| 69 Mb-70 Mb | 49 Mb-50 Mb | 199 Mb-20 Mb0 | 172 Mb-173 Mb | 24 Mb-25 Mb |
| chr10: | chr15: | chr2: | chr4: | chr8: |
| 70 Mb-71 Mb | 50 Mb-51 Mb | 20 Mb0-201 Mb | 173 Mb-174 Mb | 25 Mb-26 Mb |
| chr10: | chr15: | chr2: | chr4: | chr8: |
| 71 Mb-72 Mb | 51 Mb-52 Mb | 201 Mb-202 Mb | 174 Mb-175 Mb | 26 Mb-27 Mb |
| chr10: | chr15: | chr2: | chr4: | chr8: |
| 72 Mb-73 Mb | 52 Mb-53 Mb | 202 Mb-203 Mb | 175 Mb-176 Mb | 27 Mb-28 Mb |
| chr10: | chr15: | chr2: | chr4: | chr8: |
| 73 Mb-74 Mb | 53 Mb-54 Mb | 203 Mb-204 Mb | 176 Mb-177 Mb | 28 Mb-29 Mb |
| chr10: | chr15: | chr2: | chr4: | chr8: |
| 74 Mb-75 Mb | 54 Mb-55 Mb | 204 Mb-205 Mb | 177 Mb-178 Mb | 29 Mb-30 Mb |
| chr10: | chr15: | chr2: | chr4: | chr8: |
| 75 Mb-76 Mb | 55 Mb-56 Mb | 205 Mb-206 Mb | 178 Mb-179 Mb | 30 Mb-31 Mb |
| chr10: | chr15: | chr2: | chr4: | chr8: |
| 76 Mb-77 Mb | 56 Mb-57 Mb | 206 Mb-207 Mb | 179 Mb-18 Mb0 | 31 Mb-32 Mb |
| chr10: | chr15: | chr2: | chr4: | chr8: |
| 77 Mb-78 Mb | 57 Mb-58 Mb | 207 Mb-208 Mb | 18 Mb0-181 Mb | 32 Mb-33 Mb |
| chr10: | chr15: | chr2: | chr4: | chr8: |
| 78 Mb-79 Mb | 58 Mb-59 Mb | 208 Mb-209 Mb | 181 Mb-182 Mb | 33 Mb-34 Mb |
| chr10: | chr15: | chr2: | chr4: | chr8: |
| 79 Mb-8 Mb0 | 59 Mb-60 Mb | 209 Mb-210 Mb | 182 Mb-183 Mb | 34 Mb-35 Mb |
| chr10: | chr15: | chr2: | chr4: | chr8: |
| 8 Mb0-81 Mb | 60 Mb-61 Mb | 210 Mb-211 Mb | 183 Mb-184 Mb | 35 Mb-36 Mb |
| chr10: | chr15: | chr2: | chr4: | chr8: |
| 81 Mb-82 Mb | 61 Mb-62 Mb | 211 Mb-212 Mb | 184 Mb-185 Mb | 36 Mb-37 Mb |
| chr10: | chr15: | chr2: | chr4: | chr8: |
| 82 Mb-83 Mb | 62 Mb-63 Mb | 212 Mb-213 Mb | 185 Mb-186 Mb | 37 Mb-38 Mb |
| chr10: | chr15: | chr2: | chr4: | chr8: |
| 83 Mb-84 Mb | 63 Mb-64 Mb | 213 Mb-214 Mb | 186 Mb-187 Mb | 38 Mb-39 Mb |
| chr10: | chr15: | chr2: | chr4: | chr8: |
| 84 Mb-85 Mb | 64 Mb-65 Mb | 214 Mb-215 Mb | 187 Mb-188 Mb | 39 Mb-40 Mb |
| chr10: | chr15: | chr2: | chr4: | chr8: |
| 85 Mb-86 Mb | 65 Mb-66 Mb | 215 Mb-216 Mb | 188 Mb-189 Mb | 40 Mb-41 Mb |
| chr10: | chr15: | chr2: | chr4: | chr8: |
| 86 Mb-87 Mb | 66 Mb-67 Mb | 216 Mb-217 Mb | 189 Mb-190 Mb | 41 Mb-42 Mb |
| chr10: | chr15: | chr2: | chr4: | chr8: |
| 87 Mb-88 Mb | 67 Mb-68 Mb | 217 Mb-218 Mb | 190 Mb-191 Mb | 42 Mb-43 Mb |
| chr10: | chr15: | chr2: | chr4: | chr8: |
| 88 Mb-89 Mb | 68 Mb-69 Mb | 218 Mb-219 Mb | 191 Mb-191154276 | 43 Mb-44 Mb |
| chr10: | chr15: | chr2: | chr5: | chr8: |
| 89 Mb-90 Mb | 69 Mb-70 Mb | 219 Mb-220 Mb | 0-1 Mb | 47 Mb-48 Mb |
| chr10: | chr15: | chr2: | chr5: | chr8: |
| 90 Mb-91 Mb | 70 Mb-71 Mb | 220 Mb-221 Mb | 1 Mb-2 Mb | 48 Mb-49 Mb |
| chr10: | chr15: | chr2: | chr5: | chr8: |
| 91 Mb-92 Mb | 71 Mb-72 Mb | 221 Mb-222 Mb | 2 Mb-3 Mb | 49 Mb-50 Mb |
| chr10: | chr15: | chr2: | chr5: | chr8: |
| 92 Mb-93 Mb | 72 Mb-73 Mb | 222 Mb-223 Mb | 3 Mb-4 Mb | 50 Mb-51 Mb |
| chr10: | chr15: | chr2: | chr5: | chr8: |
| 93 Mb-94 Mb | 73 Mb-74 Mb | 223 Mb-224 Mb | 4 Mb-5 Mb | 51 Mb-52 Mb |
| chr10: | chr15: | chr2: | chr5: | chr8: |
| 94 Mb-95 Mb | 74 Mb-75 Mb | 224 Mb-225 Mb | 5 Mb-6 Mb | 52 Mb-53 Mb |
| chr10: | chr15: | chr2: | chr5: | chr8: |
| 95 Mb-96 Mb | 75 Mb-76 Mb | 225 Mb-226 Mb | 6 Mb-7 Mb | 53 Mb-54 Mb |
| chr10: | chr15: | chr2: | chr5: | chr8: |
| 96 Mb-97 Mb | 76 Mb-77 Mb | 226 Mb-227 Mb | 7 Mb-8 Mb | 54 Mb-55 Mb |
| chr10: | chr15: | chr2: | chr5: | chr8: |
| 97 Mb-98 Mb | 77 Mb-78 Mb | 227 Mb-228 Mb | 8 Mb-9 Mb | 55 Mb-56 Mb |
| chr10: | chr15: | chr2: | chr5: | chr8: |
| 98 Mb-99 Mb | 78 Mb-79 Mb | 228 Mb-229 Mb | 9 Mb-10 Mb | 56 Mb-57 Mb |
| chr10: | chr15: | chr2: | chr5: | chr8: |
| 99 Mb-100 Mb | 79 Mb-8 Mb0 | 229 Mb-230 Mb | 10 Mb-11 Mb | 57 Mb-58 Mb |
| chr10: | chr15: | chr2: | chr5: | chr8: |
| 100 Mb-101 Mb | 8 Mb0-81 Mb | 230 Mb-231 Mb | 11 Mb-12 Mb | 58 Mb-59 Mb |
| chr10: | chr15: | chr2: | chr5: | chr8: |
| 101 Mb-102 Mb | 81 Mb-82 Mb | 231 Mb-232 Mb | 12 Mb-13 Mb | 59 Mb-60 Mb |
| chr10: | chr15: | chr2: | chr5: | chr8: |
| 102 Mb-103 Mb | 82 Mb-83 Mb | 232 Mb-233 Mb | 13 Mb-14 Mb | 60 Mb-61 Mb |
| chr10: | chr15: | chr2: | chr5: | chr8: |
| 103 Mb-104 Mb | 83 Mb-84 Mb | 233 Mb-234 Mb | 14 Mb-15 Mb | 61 Mb-62 Mb |
| chr10: | chr15: | chr2: | chr5: | chr8: |
| 104 Mb-105 Mb | 84 Mb-85 Mb | 234 Mb-235 Mb | 15 Mb-16 Mb | 62 Mb-63 Mb |
| chr10: | chr15: | chr2: | chr5: | chr8: |
| 105 Mb-106 Mb | 85 Mb-86 Mb | 235 Mb-236 Mb | 16 Mb-17 Mb | 63 Mb-64 Mb |
| chr10: | chr15: | chr2: | chr5: | chr8: |
| 106 Mb-107 Mb | 86 Mb-87 Mb | 236 Mb-237 Mb | 17 Mb-18 Mb | 64 Mb-65 Mb |
| chr10: | chr15: | chr2: | chr5: | chr8: |
| 107 Mb-108 Mb | 87 Mb-88 Mb | 237 Mb-238 Mb | 18 Mb-19 Mb | 65 Mb-66 Mb |
| chr10: | chr15: | chr2: | chr5: | chr8: |
| 108 Mb-109 Mb | 88 Mb-89 Mb | 238 Mb-239 Mb | 19 Mb-20 Mb | 66 Mb-67 Mb |
| chr10: | chr15: | chr2: | chr5: | chr8: |
| 109 Mb-110 Mb | 89 Mb-90 Mb | 239 Mb-240 Mb | 20 Mb-21 Mb | 67 Mb-68 Mb |
| chr10: | chr15: | chr2: | chr5: | chr8: |
| 110 Mb-111 Mb | 90 Mb-91 Mb | 240 Mb-241 Mb | 21 Mb-22 Mb | 68 Mb-69 Mb |
| chr10: | chr15: | chr2: | chr5: | chr8: |
| 111 Mb-112 Mb | 91 Mb-92 Mb | 241 Mb-242 Mb | 22 Mb-23 Mb | 69 Mb-70 Mb |
| chr10: | chr15: | chr2: | chr5: | chr8: |
| 112 Mb-113 Mb | 92 Mb-93 Mb | 242 Mb-243 Mb | 23 Mb-24 Mb | 70 Mb-71 Mb |
| chr10: | chr15: | chr2: | chr5: | chr8: |
| 113 Mb-114 Mb | 93 Mb-94 Mb | 243 Mb-243199373 | 24 Mb-25 Mb | 71 Mb-72 Mb |
| chr10: | chr15: | chr20: | chr5: | chr8: |
| 114 Mb-115 Mb | 94 Mb-95 Mb | 0-1 Mb | 25 Mb-26 Mb | 72 Mb-73 Mb |
| chr10: | chr15: | chr20: | chr5: | chr8: |
| 115 Mb-116 Mb | 95 Mb-96 Mb | 1 Mb-2 Mb | 26 Mb-27 Mb | 73 Mb-74 Mb |
| chr10: | chr15: | chr20: | chr5: | chr8: |
| 116 Mb-117 Mb | 96 Mb-97 Mb | 2 Mb-3 Mb | 27 Mb-28 Mb | 74 Mb-75 Mb |
| chr10: | chr15: | chr20: | chr5: | chr8: |
| 117 Mb-118 Mb | 97 Mb-98 Mb | 3 Mb-4 Mb | 28 Mb-29 Mb | 75 Mb-76 Mb |
| chr10: | chr15: | chr20: | chr5: | chr8: |
| 118 Mb-119 Mb | 98 Mb-99 Mb | 4 Mb-5 Mb | 29 Mb-30 Mb | 76 Mb-77 Mb |
| chr10: | chr15: | chr20: | chr5: | chr8: |
| 119 Mb-120 Mb | 99 Mb-100 Mb | 5 Mb-6 Mb | 30 Mb-31 Mb | 77 Mb-78 Mb |
| chr10: | chr15: | chr20: | chr5: | chr8: |
| 120 Mb-121 Mb | 100 Mb-101 Mb | 6 Mb-7 Mb | 31 Mb-32 Mb | 78 Mb-79 Mb |
| chr10: | chr15: | chr20: | chr5: | chr8: |
| 121 Mb-122 Mb | 101 Mb-102 Mb | 7 Mb-8 Mb | 32 Mb-33 Mb | 79 Mb-8 Mb0 |
| chr10: | chr15: | chr20: | chr5: | chr8: |
| 122 Mb-123 Mb | 102 Mb-102531392 | 8 Mb-9 Mb | 33 Mb-34 Mb | 8 Mb0-81 Mb |
| chr10: | chr16: | chr20: | chr5: | chr8: |
| 123 Mb-124 Mb | 0-1 Mb | 9 Mb-10 Mb | 34 Mb-35 Mb | 81 Mb-82 Mb |
| chr10: | chr16: | chr20: | chr5: | chr8: |
| 124 Mb-125 Mb | 1 Mb-2 Mb | 10 Mb-11 Mb | 35 Mb-36 Mb | 82 Mb-83 Mb |
| chr10: | chr16: | chr20: | chr5: | chr8: |
| 125 Mb-126 Mb | 2 Mb-3 Mb | 11 Mb-12 Mb | 36 Mb-37 Mb | 83 Mb-84 Mb |
| chr10: | chr16: | chr20: | chr5: | chr8: |
| 126 Mb-127 Mb | 3 Mb-4 Mb | 12 Mb-13 Mb | 37 Mb-38 Mb | 84 Mb-85 Mb |
| chr10: | chr16: | chr20: | chr5: | chr8: |
| 127 Mb-128 Mb | 4 Mb-5 Mb | 13 Mb-14 Mb | 38 Mb-39 Mb | 85 Mb-86 Mb |
| chr10: | chr16: | chr20: | chr5: | chr8: |
| 128 Mb-129 Mb | 5 Mb-6 Mb | 14 Mb-15 Mb | 39 Mb-40 Mb | 86 Mb-87 Mb |
| chr10: | chr16: | chr20: | chr5: | chr8: |
| 129 Mb-130 Mb | 6 Mb-7 Mb | 15 Mb-16 Mb | 40 Mb-41 Mb | 87 Mb-88 Mb |
| chr10: | chr16: | chr20: | chr5: | chr8: |
| 130 Mb-131 Mb | 7 Mb-8 Mb | 16 Mb-17 Mb | 41 Mb-42 Mb | 88 Mb-89 Mb |
| chr10: | chr16: | chr20: | chr5: | chr8: |
| 131 Mb-132 Mb | 8 Mb-9 Mb | 17 Mb-18 Mb | 42 Mb-43 Mb | 89 Mb-90 Mb |
| chr10: | chr16: | chr20: | chr5: | chr8: |
| 132 Mb-133 Mb | 9 Mb-10 Mb | 18 Mb-19 Mb | 43 Mb-44 Mb | 90 Mb-91 Mb |
| chr10: | chr16: | chr20: | chr5: | chr8: |
| 133 Mb-134 Mb | 10 Mb-11 Mb | 19 Mb-20 Mb | 44 Mb-45 Mb | 91 Mb-92 Mb |
| chr10: | chr16: | chr20: | chr5: | chr8: |
| 134 Mb-135 Mb | 11 Mb-12 Mb | 20 Mb-21 Mb | 45 Mb-46 Mb | 92 Mb-93 Mb |
| chr10: | chr16: | chr20: | chr5: | chr8: |
| 135 Mb-135534747 | 12 Mb-13 Mb | 21 Mb-22 Mb | 49 Mb-50 Mb | 93 Mb-94 Mb |
| chr11: | chr16: | chr20: | chr5: | chr8: |
| 0-1 Mb | 13 Mb-14 Mb | 22 Mb-23 Mb | 50 Mb-51 Mb | 94 Mb-95 Mb |
| chr11: | chr16: | chr20: | chr5: | chr8: |
| 1 Mb-2 Mb | 14 Mb-15 Mb | 23 Mb-24 Mb | 51 Mb-52 Mb | 95 Mb-96 Mb |
| chr11: | chr16: | chr20: | chr5: | chr8: |
| 2 Mb-3 Mb | 15 Mb-16 Mb | 24 Mb-25 Mb | 52 Mb-53 Mb | 96 Mb-97 Mb |
| chr11: | chr16: | chr20: | chr5: | chr8: |
| 3 Mb-4 Mb | 16 Mb-17 Mb | 25 Mb-26 Mb | 53 Mb-54 Mb | 97 Mb-98 Mb |
| chr11: | chr16: | chr20: | chr5: | chr8: |
| 4 Mb-5 Mb | 17 Mb-18 Mb | 26 Mb-27 Mb | 54 Mb-55 Mb | 98 Mb-99 Mb |
| chr11: | chr16: | chr20: | chr5: | chr8: |
| 5 Mb-6 Mb | 18 Mb-19 Mb | 29 Mb-30 Mb | 55 Mb-56 Mb | 99 Mb-100 Mb |
| chr11: | chr16: | chr20: | chr5: | chr8: |
| 6 Mb-7 Mb | 19 Mb-20 Mb | 30 Mb-31 Mb | 56 Mb-57 Mb | 100 Mb-101 Mb |
| chr11: | chr16: | chr20: | chr5: | chr8: |
| 7 Mb-8 Mb | 20 Mb-21 Mb | 31 Mb-32 Mb | 57 Mb-58 Mb | 101 Mb-102 Mb |
| chr11: | chr16: | chr20: | chr5: | chr8: |
| 8 Mb-9 Mb | 21 Mb-22 Mb | 32 Mb-33 Mb | 58 Mb-59 Mb | 102 Mb-103 Mb |
| chr11: | chr16: | chr20: | chr5: | chr8: |
| 9 Mb-10 Mb | 22 Mb-23 Mb | 33 Mb-34 Mb | 59 Mb-60 Mb | 103 Mb-104 Mb |
| chr11: | chr16: | chr20: | chr5: | chr8: |
| 10 Mb-11 Mb | 23 Mb-24 Mb | 34 Mb-35 Mb | 60 Mb-61 Mb | 104 Mb-105 Mb |
| chr11: | chr16: | chr20: | chr5: | chr8: |
| 11 Mb-12 Mb | 24 Mb-25 Mb | 35 Mb-36 Mb | 61 Mb-62 Mb | 105 Mb-106 Mb |
| chr11: | chr16: | chr20: | chr5: | chr8: |
| 12 Mb-13 Mb | 25 Mb-26 Mb | 36 Mb-37 Mb | 62 Mb-63 Mb | 106 Mb-107 Mb |
| chr11: | chr16: | chr20: | chr5: | chr8: |
| 13 Mb-14 Mb | 26 Mb-27 Mb | 37 Mb-38 Mb | 63 Mb-64 Mb | 107 Mb-108 Mb |
| chr11: | chr16: | chr20: | chr5: | chr8: |
| 14 Mb-15 Mb | 27 Mb-28 Mb | 38 Mb-39 Mb | 64 Mb-65 Mb | 108 Mb-109 Mb |
| chr11: | chr16: | chr20: | chr5: | chr8: |
| 15 Mb-16 Mb | 28 Mb-29 Mb | 39 Mb-40 Mb | 65 Mb-66 Mb | 109 Mb-110 Mb |
| chr11: | chr16: | chr20: | chr5: | chr8: |
| 16 Mb-17 Mb | 29 Mb-30 Mb | 40 Mb-41 Mb | 66 Mb-67 Mb | 110 Mb-111 Mb |
| chr11: | chr16: | chr20: | chr5: | chr8: |
| 17 Mb-18 Mb | 30 Mb-31 Mb | 41 Mb-42 Mb | 67 Mb-68 Mb | 111 Mb-112 Mb |
| chr11: | chr16: | chr20: | chr5: | chr8: |
| 18 Mb-19 Mb | 31 Mb-32 Mb | 42 Mb-43 Mb | 68 Mb-69 Mb | 112 Mb-113 Mb |
| chr11: | chr16: | chr20: | chr5: | chr8: |
| 19 Mb-20 Mb | 32 Mb-33 Mb | 43 Mb-44 Mb | 69 Mb-70 Mb | 113 Mb-114 Mb |
| chr11: | chr16: | chr20: | chr5: | chr8: |
| 20 Mb-21 Mb | 33 Mb-34 Mb | 44 Mb-45 Mb | 70 Mb-71 Mb | 114 Mb-115 Mb |
| chr11: | chr16: | chr20: | chr5: | chr8: |
| 21 Mb-22 Mb | 34 Mb-35 Mb | 45 Mb-46 Mb | 71 Mb-72 Mb | 115 Mb-116 Mb |
| chr11: | chr16: | chr20: | chr5: | chr8: |
| 22 Mb-23 Mb | 35 Mb-36 Mb | 46 Mb-47 Mb | 72 Mb-73 Mb | 116 Mb-117 Mb |
| chr11: | chr16: | chr20: | chr5: | chr8: |
| 23 Mb-24 Mb | 46 Mb-47 Mb | 47 Mb-48 Mb | 73 Mb-74 Mb | 117 Mb-118 Mb |
| chr11: | chr16: | chr20: | chr5: | chr8: |
| 24 Mb-25 Mb | 47 Mb-48 Mb | 48 Mb-49 Mb | 74 Mb-75 Mb | 118 Mb-119 Mb |
| chr11: | chr16: | chr20: | chr5: | chr8: |
| 25 Mb-26 Mb | 48 Mb-49 Mb | 49 Mb-50 Mb | 75 Mb-76 Mb | 119 Mb-120 Mb |
| chr11: | chr16: | chr20: | chr5: | chr8: |
| 26 Mb-27 Mb | 49 Mb-50 Mb | 50 Mb-51 Mb | 76 Mb-77 Mb | 120 Mb-121 Mb |
| chr11: | chr16: | chr20: | chr5: | chr8: |
| 27 Mb-28 Mb | 50 Mb-51 Mb | 51 Mb-52 Mb | 77 Mb-78 Mb | 121 Mb-122 Mb |
| chr11: | chr16: | chr20: | chr5: | chr8: |
| 28 Mb-29 Mb | 51 Mb-52 Mb | 52 Mb-53 Mb | 78 Mb-79 Mb | 122 Mb-123 Mb |
| chr11: | chr16: | chr20: | chr5: | chr8: |
| 29 Mb-30 Mb | 52 Mb-53 Mb | 53 Mb-54 Mb | 79 Mb-8 Mb0 | 123 Mb-124 Mb |
| chr11: | chr16: | chr20: | chr5: | chr8: |
| 30 Mb-31 Mb | 53 Mb-54 Mb | 54 Mb-55 Mb | 8 Mb0-81 Mb | 124 Mb-125 Mb |
| chr11: | chr16: | chr20: | chr5: | chr8: |
| 31 Mb-32 Mb | 54 Mb-55 Mb | 55 Mb-56 Mb | 81 Mb-82 Mb | 125 Mb-126 Mb |
| chr11: | chr16: | chr20: | chr5: | chr8: |
| 32 Mb-33 Mb | 55 Mb-56 Mb | 56 Mb-57 Mb | 82 Mb-83 Mb | 126 Mb-127 Mb |
| chr11: | chr16: | chr20: | chr5: | chr8: |
| 33 Mb-34 Mb | 56 Mb-57 Mb | 57 Mb-58 Mb | 83 Mb-84 Mb | 127 Mb-128 Mb |
| chr11: | chr16: | chr20: | chr5: | chr8: |
| 34 Mb-35 Mb | 57 Mb-58 Mb | 58 Mb-59 Mb | 84 Mb-85 Mb | 128 Mb-129 Mb |
| chr11: | chr16: | chr20: | chr5: | chr8: |
| 35 Mb-36 Mb | 58 Mb-59 Mb | 59 Mb-60 Mb | 85 Mb-86 Mb | 129 Mb-130 Mb |
| chr11: | chr16: | chr20: | chr5: | chr8: |
| 36 Mb-37 Mb | 59 Mb-60 Mb | 60 Mb-61 Mb | 86 Mb-87 Mb | 130 Mb-131 Mb |
| chr11: | chr16: | chr20: | chr5: | chr8: |
| 37 Mb-38 Mb | 60 Mb-61 Mb | 61 Mb-62 Mb | 87 Mb-88 Mb | 131 Mb-132 Mb |
| chr11: | chr16: | chr20: | chr5: | chr8: |
| 38 Mb-39 Mb | 61 Mb-62 Mb | 62 Mb-63 Mb | 88 Mb-89 Mb | 132 Mb-133 Mb |
| chr11: | chr16: | chr21: | chr5: | chr8: |
| 39 Mb-40 Mb | 62 Mb-63 Mb | 9 Mb-10 Mb | 89 Mb-90 Mb | 133 Mb-134 Mb |
| chr11: | chr16: | chr21: | chr5: | chr8: |
| 40 Mb-41 Mb | 63 Mb-64 Mb | 10 Mb-11 Mb | 90 Mb-91 Mb | 134 Mb-135 Mb |
| chr11: | chr16: | chr21: | chr5: | chr8: |
| 41 Mb-42 Mb | 64 Mb-65 Mb | 11 Mb-12 Mb | 91 Mb-92 Mb | 135 Mb-136 Mb |
| chr11: | chr16: | chr21: | chr5: | chr8: |
| 42 Mb-43 Mb | 65 Mb-66 Mb | 14 Mb-15 Mb | 92 Mb-93 Mb | 136 Mb-137 Mb |
| chr11: | chr16: | chr21: | chr5: | chr8: |
| 43 Mb-44 Mb | 66 Mb-67 Mb | 15 Mb-16 Mb | 93 Mb-94 Mb | 137 Mb-138 Mb |
| chr11: | chr16: | chr21: | chr5: | chr8: |
| 44 Mb-45 Mb | 67 Mb-68 Mb | 16 Mb-17 Mb | 94 Mb-95 Mb | 138 Mb-139 Mb |
| chr11: | chr16: | chr21: | chr5: | chr8: |
| 45 Mb-46 Mb | 68 Mb-69 Mb | 17 Mb-18 Mb | 95 Mb-96 Mb | 139 Mb-140 Mb |
| chr11: | chr16: | chr21: | chr5: | chr8: |
| 46 Mb-47 Mb | 69 Mb-70 Mb | 18 Mb-19 Mb | 96 Mb-97 Mb | 140 Mb-141 Mb |
| chr11: | chr16: | chr21: | chr5: | chr8: |
| 47 Mb-48 Mb | 70 Mb-71 Mb | 19 Mb-20 Mb | 97 Mb-98 Mb | 141 Mb-142 Mb |
| chr11: | chr16: | chr21: | chr5: | chr8: |
| 48 Mb-49 Mb | 71 Mb-72 Mb | 20 Mb-21 Mb | 98 Mb-99 Mb | 142 Mb-143 Mb |
| chr11: | chr16: | chr21: | chr5: | chr8: |
| 49 Mb-50 Mb | 72 Mb-73 Mb | 21 Mb-22 Mb | 99 Mb-100 Mb | 143 Mb-144 Mb |
| chr11: | chr16: | chr21: | chr5: | chr8: |
| 50 Mb-51 Mb | 73 Mb-74 Mb | 22 Mb-23 Mb | 100 Mb-101 Mb | 144 Mb-145 Mb |
| chr11: | chr16: | chr21: | chr5: | chr8: |
| 51 Mb-52 Mb | 74 Mb-75 Mb | 23 Mb-24 Mb | 101 Mb-102 Mb | 145 Mb-146 Mb |
| chr11: | chr16: | chr21: | chr5: | chr8: |
| 55 Mb-56 Mb | 75 Mb-76 Mb | 24 Mb-25 Mb | 102 Mb-103 Mb | 146 Mb-146364022 |
| chr11: | chr16: | chr21: | chr5: | chr9: |
| 56 Mb-57 Mb | 76 Mb-77 Mb | 25 Mb-26 Mb | 103 Mb-104 Mb | 0-1 Mb |
| chr11: | chr16: | chr21: | chr5: | chr9: |
| 57 Mb-58 Mb | 77 Mb-78 Mb | 26 Mb-27 Mb | 104 Mb-105 Mb | 1 Mb-2 Mb |
| chr11: | chr16: | chr21: | chr5: | chr9: |
| 58 Mb-59 Mb | 78 Mb-79 Mb | 27 Mb-28 Mb | 105 Mb-106 Mb | 2 Mb-3 Mb |
| chr11: | chr16: | chr21: | chr5: | chr9: |
| 59 Mb-60 Mb | 79 Mb-8 Mb0 | 28 Mb-29 Mb | 106 Mb-107 Mb | 3 Mb-4 Mb |
| chr11: | chr16: | chr21: | chr5: | chr9: |
| 60 Mb-61 Mb | 8 Mb0-81 Mb | 29 Mb-30 Mb | 107 Mb-108 Mb | 4 Mb-5 Mb |
| chr11: | chr16: | chr21: | chr5: | chr9: |
| 61 Mb-62 Mb | 81 Mb-82 Mb | 30 Mb-31 Mb | 108 Mb-109 Mb | 5 Mb-6 Mb |
| chr11: | chr16: | chr21: | chr5: | chr9: |
| 62 Mb-63 Mb | 82 Mb-83 Mb | 31 Mb-32 Mb | 109 Mb-110 Mb | 6 Mb-7 Mb |
| chr11: | chr16: | chr21: | chr5: | chr9: |
| 63 Mb-64 Mb | 83 Mb-84 Mb | 32 Mb-33 Mb | 110 Mb-111 Mb | 7 Mb-8 Mb |
| chr11: | chr16: | chr21: | chr5: | chr9: |
| 64 Mb-65 Mb | 84 Mb-85 Mb | 33 Mb-34 Mb | 111 Mb-112 Mb | 8 Mb-9 Mb |
| chr11: | chr16: | chr21: | chr5: | chr9: |
| 65 Mb-66 Mb | 85 Mb-86 Mb | 34 Mb-35 Mb | 112 Mb-113 Mb | 9 Mb-10 Mb |
| chr11: | chr16: | chr21: | chr5: | chr9: |
| 66 Mb-67 Mb | 86 Mb-87 Mb | 35 Mb-36 Mb | 113 Mb-114 Mb | 10 Mb-11 Mb |
| chr11: | chr16: | chr21: | chr5: | chr9: |
| 67 Mb-68 Mb | 87 Mb-88 Mb | 36 Mb-37 Mb | 114 Mb-115 Mb | 11 Mb-12 Mb |
| chr11: | chr16: | chr21: | chr5: | chr9: |
| 68 Mb-69 Mb | 88 Mb-89 Mb | 37 Mb-38 Mb | 115 Mb-116 Mb | 12 Mb-13 Mb |
| chr11: | chr16: | chr21: | chr5: | chr9: |
| 69 Mb-70 Mb | 89 Mb-90 Mb | 38 Mb-39 Mb | 116 Mb-117 Mb | 13 Mb-14 Mb |
| chr11: | chr16: | chr21: | chr5: | chr9: |
| 70 Mb-71 Mb | 90 Mb-90354753 | 39 Mb-40 Mb | 117 Mb-118 Mb | 14 Mb-15 Mb |
| chr11: | chr17: | chr21: | chr5: | chr9: |
| 71 Mb-72 Mb | 0-1 Mb | 40 Mb-41 Mb | 118 Mb-119 Mb | 15 Mb-16 Mb |
| chr11: | chr17: | chr21: | chr5: | chr9: |
| 72 Mb-73 Mb | 1 Mb-2 Mb | 41 Mb-42 Mb | 119 Mb-120 Mb | 16 Mb-17 Mb |
| chr11: | chr17: | chr21: | chr5: | chr9: |
| 73 Mb-74 Mb | 2 Mb-3 Mb | 42 Mb-43 Mb | 120 Mb-121 Mb | 17 Mb-18 Mb |
| chr11: | chr17: | chr21: | chr5: | chr9: |
| 74 Mb-75 Mb | 3 Mb-4 Mb | 43 Mb-44 Mb | 121 Mb-122 Mb | 18 Mb-19 Mb |
| chr11: | chr17: | chr21: | chr5: | chr9: |
| 75 Mb-76 Mb | 4 Mb-5 Mb | 44 Mb-45 Mb | 122 Mb-123 Mb | 19 Mb-20 Mb |
| chr11: | chr17: | chr21: | chr5: | chr9: |
| 76 Mb-77 Mb | 5 Mb-6 Mb | 45 Mb-46 Mb | 123 Mb-124 Mb | 20 Mb-21 Mb |
| chr11: | chr17: | chr21: | chr5: | chr9: |
| 77 Mb-78 Mb | 6 Mb-7 Mb | 46 Mb-47 Mb | 124 Mb-125 Mb | 21 Mb-22 Mb |
| chr11: | chr17: | chr21: | chr5: | chr9: |
| 78 Mb-79 Mb | 7 Mb-8 Mb | 47 Mb-48 Mb | 125 Mb-126 Mb | 22 Mb-23 Mb |
| chr11: | chr17: | chr21: | chr5: | chr9: |
| 79 Mb-8 Mb0 | 8 Mb-9 Mb | 48 Mb-48129895 | 126 Mb-127 Mb | 23 Mb-24 Mb |
| chr11: | chr17: | chr22: | chr5: | chr9: |
| 8 Mb0-81 Mb | 9 Mb-10 Mb | 16 Mb-17 Mb | 127 Mb-128 Mb | 24 Mb-25 Mb |
| chr11: | chr17: | chr22: | chr5: | chr9: |
| 81 Mb-82 Mb | 10 Mb-11 Mb | 17 Mb-18 Mb | 128 Mb-129 Mb | 25 Mb-26 Mb |
| chr11: | chr17: | chr22: | chr5: | chr9: |
| 82 Mb-83 Mb | 11 Mb-12 Mb | 18 Mb-19 Mb | 129 Mb-130 Mb | 26 Mb-27 Mb |
| chr11: | chr17: | chr22: | chr5: | chr9: |
| 83 Mb-84 Mb | 12 Mb-13 Mb | 19 Mb-20 Mb | 130 Mb-131 Mb | 27 Mb-28 Mb |
| chr11: | chr17: | chr22: | chr5: | chr9: |
| 84 Mb-85 Mb | 13 Mb-14 Mb | 20 Mb-21 Mb | 131 Mb-132 Mb | 28 Mb-29 Mb |
| chr11: | chr17: | chr22: | chr5: | chr9: |
| 85 Mb-86 Mb | 14 Mb-15 Mb | 21 Mb-22 Mb | 132 Mb-133 Mb | 29 Mb-30 Mb |
| chr11: | chr17: | chr22: | chr5: | chr9: |
| 86 Mb-87 Mb | 15 Mb-16 Mb | 22 Mb-23 Mb | 133 Mb-134 Mb | 30 Mb-31 Mb |
| chr11: | chr17: | chr22: | chr5: | chr9: |
| 87 Mb-88 Mb | 16 Mb-17 Mb | 23 Mb-24 Mb | 134 Mb-135 Mb | 31 Mb-32 Mb |
| chr11: | chr17: | chr22: | chr5: | chr9: |
| 88 Mb-89 Mb | 17 Mb-18 Mb | 24 Mb-25 Mb | 135 Mb-136 Mb | 32 Mb-33 Mb |
| chr11: | chr17: | chr22: | chr5: | chr9: |
| 89 Mb-90 Mb | 18 Mb-19 Mb | 25 Mb-26 Mb | 136 Mb-137 Mb | 33 Mb-34 Mb |
| chr11: | chr17: | chr22: | chr5: | chr9: |
| 90 Mb-91 Mb | 19 Mb-20 Mb | 26 Mb-27 Mb | 137 Mb-138 Mb | 34 Mb-35 Mb |
| chr11: | chr17: | chr22: | chr5: | chr9: |
| 91 Mb-92 Mb | 20 Mb-21 Mb | 27 Mb-28 Mb | 138 Mb-139 Mb | 35 Mb-36 Mb |
| chr11: | chr17: | chr22: | chr5: | chr9: |
| 92 Mb-93 Mb | 21 Mb-22 Mb | 28 Mb-29 Mb | 139 Mb-140 Mb | 36 Mb-37 Mb |
| chr11: | chr17: | chr22: | chr5: | chr9: |
| 93 Mb-94 Mb | 22 Mb-23 Mb | 29 Mb-30 Mb | 140 Mb-141 Mb | 37 Mb-38 Mb |
| chr11: | chr17: | chr22: | chr5: | chr9: |
| 94 Mb-95 Mb | 25 Mb-26 Mb | 30 Mb-31 Mb | 141 Mb-142 Mb | 38 Mb-39 Mb |
| chr11: | chr17: | chr22: | chr5: | chr9: |
| 95 Mb-96 Mb | 26 Mb-27 Mb | 31 Mb-32 Mb | 142 Mb-143 Mb | 39 Mb-40 Mb |
| chr11: | chr17: | chr22: | chr5: | chr9: |
| 96 Mb-97 Mb | 27 Mb-28 Mb | 32 Mb-33 Mb | 143 Mb-144 Mb | 40 Mb-41 Mb |
| chr11: | chr17: | chr22: | chr5: | chr9: |
| 97 Mb-98 Mb | 28 Mb-29 Mb | 33 Mb-34 Mb | 144 Mb-145 Mb | 41 Mb-42 Mb |
| chr11: | chr17: | chr22: | chr5: | chr9: |
| 98 Mb-99 Mb | 29 Mb-30 Mb | 34 Mb-35 Mb | 145 Mb-146 Mb | 42 Mb-43 Mb |
| chr11: | chr17: | chr22: | chr5: | chr9: |
| 99 Mb-100 Mb | 30 Mb-31 Mb | 35 Mb-36 Mb | 146 Mb-147 Mb | 43 Mb-44 Mb |
| chr11: | chr17: | chr22: | chr5: | chr9: |
| 100 Mb-101 Mb | 31 Mb-32 Mb | 36 Mb-37 Mb | 147 Mb-148 Mb | 44 Mb-45 Mb |
| chr11: | chr17: | chr22: | chr5: | chr9: |
| 101 Mb-102 Mb | 32 Mb-33 Mb | 37 Mb-38 Mb | 148 Mb-149 Mb | 45 Mb-46 Mb |
| chr11: | chr17: | chr22: | chr5: | chr9: |
| 102 Mb-103 Mb | 33 Mb-34 Mb | 38 Mb-39 Mb | 149 Mb-150 Mb | 46 Mb-47 Mb |
| chr11: | chr17: | chr22: | chr5: | chr9: |
| 103 Mb-104 Mb | 34 Mb-35 Mb | 39 Mb-40 Mb | 150 Mb-151 Mb | 47 Mb-48 Mb |
| chr11: | chr17: | chr22: | chr5: | chr9: |
| 104 Mb-105 Mb | 35 Mb-36 Mb | 40 Mb-41 Mb | 151 Mb-152 Mb | 65 Mb-66 Mb |
| chr11: | chr17: | chr22: | chr5: | chr9: |
| 105 Mb-106 Mb | 36 Mb-37 Mb | 41 Mb-42 Mb | 152 Mb-153 Mb | 66 Mb-67 Mb |
| chr11: | chr17: | chr22: | chr5: | chr9: |
| 106 Mb-107 Mb | 37 Mb-38 Mb | 42 Mb-43 Mb | 153 Mb-154 Mb | 67 Mb-68 Mb |
| chr11: | chr17: | chr22: | chr5: | chr9: |
| 107 Mb-108 Mb | 38 Mb-39 Mb | 43 Mb-44 Mb | 154 Mb-155 Mb | 68 Mb-69 Mb |
| chr11: | chr17: | chr22: | chr5: | chr9: |
| 108 Mb-109 Mb | 39 Mb-40 Mb | 44 Mb-45 Mb | 155 Mb-156 Mb | 69 Mb-70 Mb |
| chr11: | chr17: | chr22: | chr5: | chr9: |
| 109 Mb-110 Mb | 40 Mb-41 Mb | 45 Mb-46 Mb | 156 Mb-157 Mb | 70 Mb-71 Mb |
| chr11: | chr17: | chr22: | chr5: | chr9: |
| 110 Mb-111 Mb | 41 Mb-42 Mb | 46 Mb-47 Mb | 157 Mb-158 Mb | 71 Mb-72 Mb |
| chr11: | chr17: | chr22: | chr5: | chr9: |
| 111 Mb-112 Mb | 42 Mb-43 Mb | 47 Mb-48 Mb | 158 Mb-159 Mb | 72 Mb-73 Mb |
| chr11: | chr17: | chr22: | chr5: | chr9: |
| 112 Mb-113 Mb | 43 Mb-44 Mb | 48 Mb-49 Mb | 159 Mb-160 Mb | 73 Mb-74 Mb |
| chr11: | chr17: | chr22: | chr5: | chr9: |
| 113 Mb-114 Mb | 44 Mb-45 Mb | 49 Mb-50 Mb | 160 Mb-161 Mb | 74 Mb-75 Mb |
| chr11: | chr17: | chr22: | chr5: | chr9: |
| 114 Mb-115 Mb | 45 Mb-46 Mb | 50 Mb-51 Mb | 161 Mb-162 Mb | 75 Mb-76 Mb |
| chr11: | chr17: | chr22: | chr5: | chr9: |
| 115 Mb-116 Mb | 46 Mb-47 Mb | 51 Mb-51304566 | 162 Mb-163 Mb | 76 Mb-77 Mb |
| chr11: | chr17: | chr3: | chr5: | chr9: |
| 116 Mb-117 Mb | 47 Mb-48 Mb | 0-1 Mb | 163 Mb-164 Mb | 77 Mb-78 Mb |
| chr11: | chr17: | chr3: | chr5: | chr9: |
| 117 Mb-118 Mb | 48 Mb-49 Mb | 1 Mb-2 Mb | 164 Mb-165 Mb | 78 Mb-79 Mb |
| chr11: | chr17: | chr3: | chr5: | chr9: |
| 118 Mb-119 Mb | 49 Mb-50 Mb | 2 Mb-3 Mb | 165 Mb-166 Mb | 79 Mb-8 Mb0 |
| chr11: | chr17: | chr3: | chr5: | chr9: |
| 119 Mb-120 Mb | 50 Mb-51 Mb | 3 Mb-4 Mb | 166 Mb-167 Mb | 8 Mb0-81 Mb |
| chr11: | chr17: | chr3: | chr5: | chr9: |
| 120 Mb-121 Mb | 51 Mb-52 Mb | 4 Mb-5 Mb | 167 Mb-168 Mb | 81 Mb-82 Mb |
| chr11: | chr17: | chr3: | chr5: | chr9: |
| 121 Mb-122 Mb | 52 Mb-53 Mb | 5 Mb-6 Mb | 168 Mb-169 Mb | 82 Mb-83 Mb |
| chr11: | chr17: | chr3: | chr5: | chr9: |
| 122 Mb-123 Mb | 53 Mb-54 Mb | 6 Mb-7 Mb | 169 Mb-170 Mb | 83 Mb-84 Mb |
| chr11: | chr17: | chr3: | chr5: | chr9: |
| 123 Mb-124 Mb | 54 Mb-55 Mb | 7 Mb-8 Mb | 170 Mb-171 Mb | 84 Mb-85 Mb |
| chr11: | chr17: | chr3: | chr5: | chr9: |
| 124 Mb-125 Mb | 55 Mb-56 Mb | 8 Mb-9 Mb | 171 Mb-172 Mb | 85 Mb-86 Mb |
| chr11: | chr17: | chr3: | chr5: | chr9: |
| 125 Mb-126 Mb | 56 Mb-57 Mb | 9 Mb-10 Mb | 172 Mb-173 Mb | 86 Mb-87 Mb |
| chr11: | chr17: | chr3: | chr5: | chr9: |
| 126 Mb-127 Mb | 57 Mb-58 Mb | 10 Mb-11 Mb | 173 Mb-174 Mb | 87 Mb-88 Mb |
| chr11: | chr17: | chr3: | chr5: | chr9: |
| 127 Mb-128 Mb | 58 Mb-59 Mb | 11 Mb-12 Mb | 174 Mb-175 Mb | 88 Mb-89 Mb |
| chr11: | chr17: | chr3: | chr5: | chr9: |
| 128 Mb-129 Mb | 59 Mb-60 Mb | 12 Mb-13 Mb | 175 Mb-176 Mb | 89 Mb-90 Mb |
| chr11: | chr17: | chr3: | chr5: | chr9: |
| 129 Mb-130 Mb | 60 Mb-61 Mb | 13 Mb-14 Mb | 176 Mb-177 Mb | 90 Mb-91 Mb |
| chr11: | chr17: | chr3: | chr5: | chr9: |
| 130 Mb-131 Mb | 61 Mb-62 Mb | 14 Mb-15 Mb | 177 Mb-178 Mb | 91 Mb-92 Mb |
| chr11: | chr17: | chr3: | chr5: | chr9: |
| 131 Mb-132 Mb | 62 Mb-63 Mb | 15 Mb-16 Mb | 178 Mb-179 Mb | 92 Mb-93 Mb |
| chr11: | chr17: | chr3: | chr5: | chr9: |
| 132 Mb-133 Mb | 63 Mb-64 Mb | 16 Mb-17 Mb | 179 Mb-18 Mb0 | 93 Mb-94 Mb |
| chr11: | chr17: | chr3: | chr5: | chr9: |
| 133 Mb-134 Mb | 64 Mb-65 Mb | 17 Mb-18 Mb | 18 Mb0-180915260 | 94 Mb-95 Mb |
| chr11: | chr17: | chr3: | chr6: | chr9: |
| 134 Mb-135 Mb | 65 Mb-66 Mb | 18 Mb-19 Mb | 0-1 Mb | 95 Mb-96 Mb |
| chr12: | chr17: | chr3: | chr6: | chr9: |
| 0-1 Mb | 66 Mb-67 Mb | 19 Mb-20 Mb | 1 Mb-2 Mb | 96 Mb-97 Mb |
| chr12: | chr17: | chr3: | chr6: | chr9: |
| 1 Mb-2 Mb | 67 Mb-68 Mb | 20 Mb-21 Mb | 2 Mb-3 Mb | 97 Mb-98 Mb |
| chr12: | chr17: | chr3: | chr6: | chr9: |
| 2 Mb-3 Mb | 68 Mb-69 Mb | 21 Mb-22 Mb | 3 Mb-4 Mb | 98 Mb-99 Mb |
| chr12: | chr17: | chr3: | chr6: | chr9: |
| 3 Mb-4 Mb | 69 Mb-70 Mb | 22 Mb-23 Mb | 4 Mb-5 Mb | 99 Mb-100 Mb |
| chr12: | chr17: | chr3: | chr6: | chr9: |
| 4 Mb-5 Mb | 70 Mb-71 Mb | 23 Mb-24 Mb | 5 Mb-6 Mb | 100 Mb-101 Mb |
| chr12: | chr17: | chr3: | chr6: | chr9: |
| 5 Mb-6 Mb | 71 Mb-72 Mb | 24 Mb-25 Mb | 6 Mb-7 Mb | 101 Mb-102 Mb |
| chr12: | chr17: | chr3: | chr6: | chr9: |
| 6 Mb-7 Mb | 72 Mb-73 Mb | 25 Mb-26 Mb | 7 Mb-8 Mb | 102 Mb-103 Mb |
| chr12: | chr17: | chr3: | chr6: | chr9: |
| 7 Mb-8 Mb | 73 Mb-74 Mb | 26 Mb-27 Mb | 8 Mb-9 Mb | 103 Mb-104 Mb |
| chr12: | chr17: | chr3: | chr6: | chr9: |
| 8 Mb-9 Mb | 74 Mb-75 Mb | 27 Mb-28 Mb | 9 Mb-10 Mb | 104 Mb-105 Mb |
| chr12: | chr17: | chr3: | chr6: | chr9: |
| 9 Mb-10 Mb | 75 Mb-76 Mb | 28 Mb-29 Mb | 10 Mb-11 Mb | 105 Mb-106 Mb |
| chr12: | chr17: | chr3: | chr6: | chr9: |
| 10 Mb-11 Mb | 76 Mb-77 Mb | 29 Mb-30 Mb | 11 Mb-12 Mb | 106 Mb-107 Mb |
| chr12: | chr17: | chr3: | chr6: | chr9: |
| 11 Mb-12 Mb | 77 Mb-78 Mb | 30 Mb-31 Mb | 12 Mb-13 Mb | 107 Mb-108 Mb |
| chr12: | chr17: | chr3: | chr6: | chr9: |
| 12 Mb-13 Mb | 78 Mb-79 Mb | 31 Mb-32 Mb | 13 Mb-14 Mb | 108 Mb-109 Mb |
| chr12: | chr17: | chr3: | chr6: | chr9: |
| 13 Mb-14 Mb | 79 Mb-8 Mb0 | 32 Mb-33 Mb | 14 Mb-15 Mb | 109 Mb-110 Mb |
| chr12: | chr17: | chr3: | chr6: | chr9: |
| 14 Mb-15 Mb | 8 Mb0-81 Mb | 33 Mb-34 Mb | 15 Mb-16 Mb | 110 Mb-111 Mb |
| chr12: | chr17: | chr3: | chr6: | chr9: |
| 15 Mb-16 Mb | 81 Mb-81195210 | 34 Mb-35 Mb | 16 Mb-17 Mb | 111 Mb-112 Mb |
| chr12: | chr18: | chr3: | chr6: | chr9: |
| 16 Mb-17 Mb | 0-1 Mb | 35 Mb-36 Mb | 17 Mb-18 Mb | 112 Mb-113 Mb |
| chr12: | chr18: | chr3: | chr6: | chr9: |
| 17 Mb-18 Mb | 1 Mb-2 Mb | 36 Mb-37 Mb | 18 Mb-19 Mb | 113 Mb-114 Mb |
| chr12: | chr18: | chr3: | chr6: | chr9: |
| 18 Mb-19 Mb | 2 Mb-3 Mb | 37 Mb-38 Mb | 19 Mb-20 Mb | 114 Mb-115 Mb |
| chr12: | chr18: | chr3: | chr6: | chr9: |
| 19 Mb-20 Mb | 3 Mb-4 Mb | 38 Mb-39 Mb | 20 Mb-21 Mb | 115 Mb-116 Mb |
| chr12: | chr18: | chr3: | chr6: | chr9: |
| 20 Mb-21 Mb | 4 Mb-5 Mb | 39 Mb-40 Mb | 21 Mb-22 Mb | 116 Mb-117 Mb |
| chr12: | chr18: | chr3: | chr6: | chr9: |
| 21 Mb-22 Mb | 5 Mb-6 Mb | 40 Mb-41 Mb | 22 Mb-23 Mb | 117 Mb-118 Mb |
| chr12: | chr18: | chr3: | chr6: | chr9: |
| 22 Mb-23 Mb | 6 Mb-7 Mb | 41 Mb-42 Mb | 23 Mb-24 Mb | 118 Mb-119 Mb |
| chr12: | chr18: | chr3: | chr6: | chr9: |
| 23 Mb-24 Mb | 7 Mb-8 Mb | 42 Mb-43 Mb | 24 Mb-25 Mb | 119 Mb-120 Mb |
| chr12: | chr18: | chr3: | chr6: | chr9: |
| 24 Mb-25 Mb | 8 Mb-9 Mb | 43 Mb-44 Mb | 25 Mb-26 Mb | 120 Mb-121 Mb |
| chr12: | chr18: | chr3: | chr6: | chr9: |
| 25 Mb-26 Mb | 9 Mb-10 Mb | 44 Mb-45 Mb | 26 Mb-27 Mb | 121 Mb-122 Mb |
| chr12: | chr18: | chr3: | chr6: | chr9: |
| 26 Mb-27 Mb | 10 Mb-11 Mb | 45 Mb-46 Mb | 27 Mb-28 Mb | 122 Mb-123 Mb |
| chr12: | chr18: | chr3: | chr6: | chr9: |
| 27 Mb-28 Mb | 11 Mb-12 Mb | 46 Mb-47 Mb | 28 Mb-29 Mb | 123 Mb-124 Mb |
| chr12: | chr18: | chr3: | chr6: | chr9: |
| 28 Mb-29 Mb | 12 Mb-13 Mb | 47 Mb-48 Mb | 29 Mb-30 Mb | 124 Mb-125 Mb |
| chr12: | chr18: | chr3: | chr6: | chr9: |
| 29 Mb-30 Mb | 13 Mb-14 Mb | 48 Mb-49 Mb | 31 Mb-32 Mb | 125 Mb-126 Mb |
| chr12: | chr18: | chr3: | chr6: | chr9: |
| 30 Mb-31 Mb | 14 Mb-15 Mb | 49 Mb-50 Mb | 32 Mb-33 Mb | 126 Mb-127 Mb |
| chr12: | chr18: | chr3: | chr6: | chr9: |
| 31 Mb-32 Mb | 15 Mb-16 Mb | 50 Mb-51 Mb | 33 Mb-34 Mb | 127 Mb-128 Mb |
| chr12: | chr18: | chr3: | chr6: | chr9: |
| 32 Mb-33 Mb | 18 Mb-19 Mb | 51 Mb-52 Mb | 34 Mb-35 Mb | 128 Mb-129 Mb |
| chr12: | chr18: | chr3: | chr6: | chr9: |
| 33 Mb-34 Mb | 19 Mb-20 Mb | 52 Mb-53 Mb | 35 Mb-36 Mb | 129 Mb-130 Mb |
| chr12: | chr18: | chr3: | chr6: | chr9: |
| 34 Mb-35 Mb | 20 Mb-21 Mb | 53 Mb-54 Mb | 36 Mb-37 Mb | 130 Mb-131 Mb |
| chr12: | chr18: | chr3: | chr6: | chr9: |
| 37 Mb-38 Mb | 21 Mb-22 Mb | 54 Mb-55 Mb | 37 Mb-38 Mb | 131 Mb-132 Mb |
| chr12: | chr18: | chr3: | chr6: | chr9: |
| 38 Mb-39 Mb | 22 Mb-23 Mb | 55 Mb-56 Mb | 38 Mb-39 Mb | 132 Mb-133 Mb |
| chr12: | chr18: | chr3: | chr6: | chr9: |
| 39 Mb-40 Mb | 23 Mb-24 Mb | 56 Mb-57 Mb | 39 Mb-40 Mb | 133 Mb-134 Mb |
| chr12: | chr18: | chr3: | chr6: | chr9: |
| 40 Mb-41 Mb | 24 Mb-25 Mb | 57 Mb-58 Mb | 40 Mb-41 Mb | 134 Mb-135 Mb |
| chr12: | chr18: | chr3: | chr6: | chr9: |
| 41 Mb-42 Mb | 25 Mb-26 Mb | 58 Mb-59 Mb | 41 Mb-42 Mb | 135 Mb-136 Mb |
| chr12: | chr18: | chr3: | chr6: | chr9: |
| 42 Mb-43 Mb | 26 Mb-27 Mb | 59 Mb-60 Mb | 42 Mb-43 Mb | 136 Mb-137 Mb |
| chr12: | chr18: | chr3: | chr6: | chr9: |
| 43 Mb-44 Mb | 27 Mb-28 Mb | 60 Mb-61 Mb | 43 Mb-44 Mb | 137 Mb-138 Mb |
| chr12: | chr18: | chr3: | chr6: | chr9: |
| 44 Mb-45 Mb | 28 Mb-29 Mb | 61 Mb-62 Mb | 44 Mb-45 Mb | 138 Mb-139 Mb |
| chr12: | chr18: | chr3: | chr6: | chr9: |
| 45 Mb-46 Mb | 29 Mb-30 Mb | 62 Mb-63 Mb | 45 Mb-46 Mb | 139 Mb-140 Mb |
| chr12: | chr18: | chr3: | chr6: | chr9: |
| 46 Mb-47 Mb | 30 Mb-31 Mb | 63 Mb-64 Mb | 46 Mb-47 Mb | 140 Mb-141 Mb |
| chr12: | chr18: | chr3: | chr6: | chr9: |
| 47 Mb-48 Mb | 31 Mb-32 Mb | 64 Mb-65 Mb | 47 Mb-48 Mb | 141 Mb-141213431 |
| chr12: | chr18: | chr3: | | |
| 48 Mb-49 Mb | 32 Mb-33 Mb | 65 Mb-66 Mb | | |
| chr12: | chr18: | chr3: | | |
| 49 Mb-50 Mb | 33 Mb-34 Mb | 66 Mb-67 Mb | | |
| chr12: | chr18: | chr3: | | |
| 50 Mb-51 Mb | 34 Mb-35 Mb | 67 Mb-68 Mb | | |
| chr12: | chr18: | chr3: | | |
| 51 Mb-52 Mb | 35 Mb-36 Mb | 68 Mb-69 Mb | | |

### 2-3. Calculation of the Frequency of Single Nucleotide Variants Mutation Signature

The frequency of single nucleotide variants mutation signature in the entire genome was calculated in the entire genome. There are four criteria for dividing the types of mutations.

First, a total of six basic mutations was defined (C>A, C>G, C>T, T>A, T>C, T>G) depending on the type of the reference base and the substituted base. Second, 24 (4×6) mutation types were defined in consideration of one 5′ direction base in addition to the basic mutation types. Third, 24 (6×4) mutation types were defined in consideration of one 3′ direction base in addition to the basic mutation types. Finally, 96 (4×6×4) mutation types, which are commonly used in the mutation signature analysis, were determined in consideration of one 5′ direction base and one 3′ direction base in addition to the basic mutation types.

The frequency of occurrence was calculated in each of a total of 150 mutation types. In addition, the sum of the number of mutations was calculated for each of the four mutation classification methods and then was divided by the sum of all mutations generated in the entire base, and normalization was conducted.

The types of mutations are defined as shown in Table 2 below.

TABLE 2
List of mutation types

| | | | | | | | | | |
|---|---|---|---|---|---|---|---|---|---|
| sig- | C > A | sig-rev | 3CA:C > | sig-tri | TCG:C > | sig-tri | TCA:C > | sig-tri | ATG:T > |
| one | | | A | | A | | T | | C |
| sig- | C > G | sig-rev | 3CT:C > | sig-tri | TCC:C > | sig-tri | TCT:C > | sig-tri | ATC:T > |
| one | | | A | | A | | T | | C |
| sig- | C > T | sig-rev | 3CG:C > | sig-tri | GCA:C > | sig-tri | TCG:C > | sig-tri | TTA:T > |
| one | | | A | | A | | T | | C |
| sig- | T > A | sig-rev | 3CC:C > | sig-tri | GCT:C > | sig-tri | TCC:C > | sig-tri | TTT:T > |
| one | | | A | | A | | T | | C |
| sig- | T > C | sig-rev | 3CA:C > | sig-tri | GCG:C > | sig-tri | GCA:C > | sig-tri | TTG:T > |
| one | | | G | | A | | T | | C |
| sig- | T > G | sig-rev | 3CT:C > | sig-tri | GCC:C > | sig-tri | GCT:C > | sig-tri | TTC:T > |
| one | | | G | | A | | T | | C |
| sig- | 5AC:C > | sig-rev | 3CG:C > | sig-tri | CCA:C > | sig-tri | GCG:C > | sig-tri | GTA:T > |
| for | A | | G | | A | | T | | C |
| sig- | 5TC:C > | sig-rev | 3CC:C > | sig-tri | CCT:C > | sig-tri | GCC:C > | sig-tri | GTT:T > |
| for | A | | G | | A | | T | | C |
| sig- | 5GC:C > | sig-rev | 3CA:C > | sig-tri | CCG:C > | sig-tri | CCA:C > | sig-tri | GTG:T > |
| for | A | | T | | A | | T | | C |
| sig- | 5CC:C > | sig-rev | 3CT:C > | sig-tri | CCC:C > | sig-tri | CCT:C > | sig-tri | GTC:T > |
| for | A | | T | | A | | T | | C |
| sig- | 5AC:C > | sig-rev | 3CG:C > | sig-tri | ACA:C > | sig-tri | CCG:C > | sig-tri | CTA:T > |
| for | G | | T | | G | | T | | C |
| sig- | 5TC:C > | sig-rev | 3CC:C > | sig-tri | ACT:C > | sig-tri | CCC:C > | sig-tri | CTT:T > |
| for | G | | T | | G | | T | | C |
| sig- | 5GC:C > | sig-rev | 3TA:T > | sig-tri | ACG:C > | sig-tri | ATA:T > | sig-tri | CTG:T > |
| for | G | | A | | G | | A | | C |
| sig- | 5CC:C > | sig-rev | 3TT:T > | sig-tri | ACC:C > | sig-tri | ATT:T > | sig-tri | CTC:T > |
| for | G | | A | | G | | A | | C |
| sig- | 5AC:C > | sig-rev | 3TG:T > | sig-tri | TCA:C > | sig-tri | ATG:T > | sig-tri | ATA:T > |
| for | T | | A | | G | | A | | G |
| sig- | 5TC:C > | sig-rev | 3TC:T > | sig-tri | TCT:C > | sig-tri | ATC:T > | sig-tri | ATT:T > |
| for | T | | A | | G | | A | | G |
| sig- | 5GC:C > | sig-rev | 3TA:T > | sig-tri | TCG:C > | sig-tri | TTA:T > | sig-tri | ATG:T > |
| for | T | | C | | G | | A | | G |
| sig- | 5CC:C > | sig-rev | 3TT:T > | sig-tri | TCC:C > | sig-tri | TTT:T > | sig-tri | ATC:T > |
| for | T | | C | | G | | A | | G |
| sig- | 5AT:T > | sig-rev | 3TG:T > | sig-tri | GCA:C > | sig-tri | TTG:T > | sig-tri | TTA:T > |
| for | A | | C | | G | | A | | G |
| sig- | 5TT:T > | sig-rev | 3TC:T > | sig-tri | GCT:C > | sig-tri | TTC:T > | sig-tri | TTT:T > |
| for | A | | C | | G | | A | | G |
| sig- | 5GT:T > | sig-rev | 3TA:T > | sig-tri | GCG:C > | sig-tri | GTA:T > | sig-tri | TTG:T > |
| for | A | | G | | G | | A | | G |
| sig- | 5CT:T > | sig-rev | 3TT:T > | sig-tri | GCC:C > | sig-tri | GTT:T > | sig-tri | TTC:T > |
| for | A | | G | | G | | A | | G |
| sig- | 5AT:T > | sig-rev | 3TG:T > | sig-tri | CCA:C > | sig-tri | GTG:T > | sig-tri | GTA:T > |
| for | C | | G | | G | | A | | G |
| sig- | 5TT:T > | sig-rev | 3TC:T > | sig-tri | CCT:C > | sig-tri | GTC:T > | sig-tri | GTT:T > |
| for | C | | G | | G | | A | | G |
| sig- | 5GT:T > | sig-tri | ACA:C > | sig-tri | CCG:C > | sig-tri | CTA:T > | sig-tri | GTG:T > |
| for | C | | A | | G | | A | | G |
| sig- | 5CT:T > | sig-tri | ACT:C > | sig-tri | CCC:C > | sig-tri | CTT:T > | sig-tri | GTC:T > |
| for | C | | A | | G | | A | | G |
| sig- | 5AT:T > | sig-tri | ACG:C > | sig-tri | ACA:C > | sig-tri | CTG:T > | sig-tri | CTA:T > |
| for | G | | A | | T | | A | | G |
| sig- | 5TT:T > | sig-tri | ACC:C > | sig-tri | ACT:C > | sig-tri | CTC:T > | sig-tri | CTT:T > |
| for | G | | A | | T | | A | | G |
| sig- | 5GT:T > | sig-tri | TCA:C > | sig-tri | ACG:C > | sig-tri | ATA:T > | sig-tri | CTG:T > |
| for | G | | A | | T | | C | | G |
| sig- | 5CT:T > | sig-tri | TCT:C > | sig-tri | ACC:C > | sig-tri | ATT:T > | sig-tri | CTC:T > |
| for | G | | A | | T | | C | | G |

Finally, 2876 single nucleotide variant features obtained by combining 2726 single nucleotide variant features with 150 single nucleotide variant features were used as input values of the algorithm.

### Example 3. First DNN Model Construction and Learning Process

A total of 2876 features for the distribution and type of single nucleotide variations obtained through the previous analysis was used to develop an algorithm for diagnosing cancer and detecting cancer types in cfDNA. A total of two artificial intelligence algorithms was developed.

First, a binary classification model to determine whether a normal subject or a cancer patient was constructed. Second, a multiple classification model to classify cancer types was constructed. As the loss functions for algorithm learning, binary cross entropy was used for the binary classification model, and categorical cross entropy was used for the multiple classification model. A deep neural network artificial intelligence model was used for algorithm learning.

All datasets were classified into train, valid, and test datasets and the model was trained using hyper-parameter tuning using a method called “Bayesian optimization”. The datasets were classified into five train, valid, and test sets, and training was performed five times to construct five algorithm models. In addition, predictions were performed on each of the five test datasets of each of the five algorithm models to use each of the datasets once as the test dataset. In this way, the performance of the model was evaluated using the prediction probability when the entire sample was the test dataset.

### Example 4. Construction of First Artificial Intelligence Model and Confirmation of Performance

In order to test the performance of the deep learning model constructed using the reads obtained in Example 1, a comparison model based on the fragmentation pattern and copy number variation (CNV) to diagnose cancer and identify cancer types based on the dataset of Example 1 was constructed for application to the cfDNA using the method of conventional artificial intelligence models used to diagnose cancer and detect cancer types (Cristiano, S. et al., Nature, Vol. 570 (7761), pp. 385-389. 2019).

More specifically, in accordance with the fragment pattern method, the entire genome was divided into 5 MB bins after GC correction, and the ratio of short fragments to the total number of fragments for each bin was used as an input value after z-score normalization. The short fragment used herein means a fragment having a length of 100 bp to 150 bp. In accordance with the CNV method, the entire genome was divided into 50 kB bins that did not overlap with one another, subjected to GC correction, and the depth was calculated for each bin and converted to the log 2 value, which was used as an input value. Xgboost was used to train fragment patterns and CNV models.

To compare the performance of cancer diagnosis models, the sensitivity at the predictive probability threshold was detected when the specificity was 95%, 98%, and 99%.

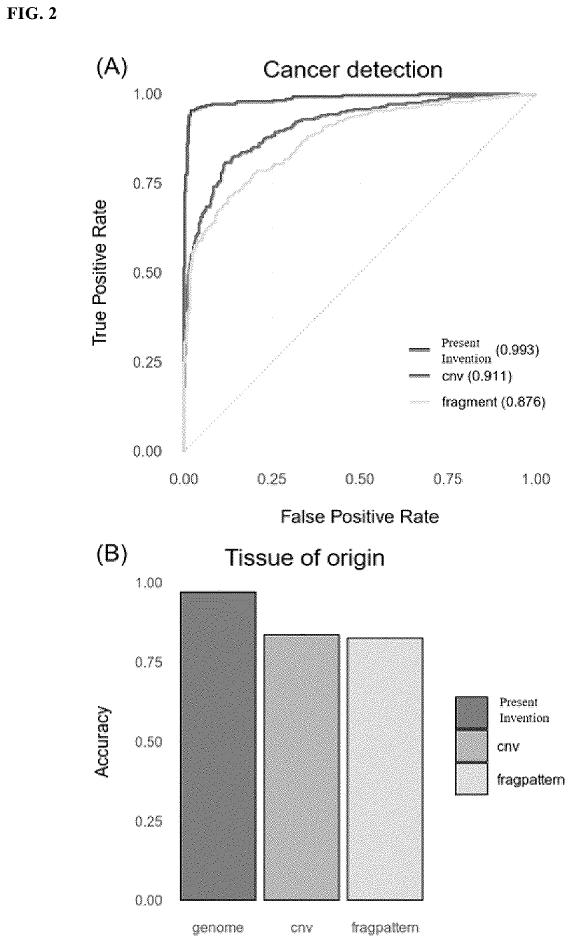

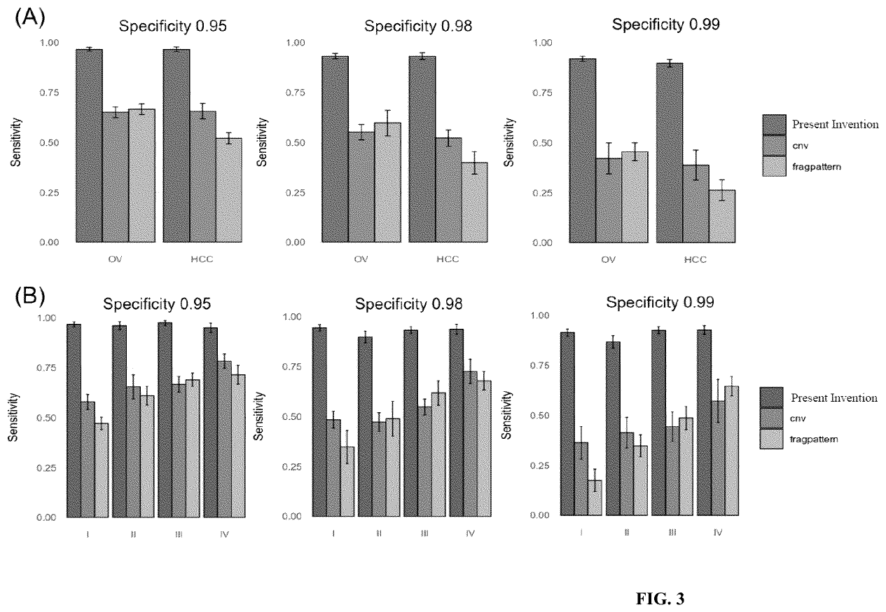

As can be seen from FIG. 2, the performance of the cancer diagnosis model constructed in the present invention was superior to that of the conventional method, as can be seen from FIG. 3, the cancer diagnosis model constructed in the present invention has excellent performance in cancer diagnosis at all accuracies, and, as can be seen from FIG. 3 in (B), the performance of the conventional method is deteriorated in early cancer diagnosis (stage I), whereas the cancer diagnosis model constructed in the present invention exhibits excellent performance even in early cancer diagnosis.

In addition, as can be seen from FIG. 4, the result of comparison in the performance between the cancer type discrimination models showed that the cancer type discrimination model constructed in the present invention exhibited excellent cancer type discrimination performance at all stages compared to the conventional method.

### Example 5. Extraction of DNA from Blood to Perform Next-Generation Sequencing to Construct Second Artificial Intelligence Model

10 mL of blood was collected from each of 202 normal subjects and 64 neuroblastoma patients, and stored in an EDTA tube. Within 2 hours after blood collection, only the plasma was primarily centrifuged at 1,200 g and 4° C. for 15 minutes, and then the primarily centrifuged plasma was secondarily centrifuged at 16,000 g and 4° C. for 10 minutes to isolate the plasma supernatant excluding the precipitate. Cell-free DNA was extracted from the isolated plasma using a TIANamp Micro DNA Kit (Tiangen), a library preparation process was performed using a MGIEasy cell-free DNA library prep set kit, and then sequencing was performed in a 100 base paired end mode using a DNBseq G400 device (MGI). As a result, about 170 million reads were found to be produced from each sample.

The produced datasets are shown in Table 4 below.

TABLE 4

| Sample Type | Train | Validation | Test | Total |
|---|---|---|---|---|
| Normal | 99 | 42 | 61 | 202 |
| Cancer | 30 | 13 | 18 | 61 |

### Example 6. Selection of Nucleic Acid Fragment End Motif and Nucleic Acid Fragment Size

### 6-1. Selection of Nucleic Acid Fragment End Motif

The nucleic acid fragment end motifs were determined from 4 bases (A, T, G, C), and among a total of 256 (4*4*4*4) motifs, some motifs had no relative frequency difference between normal and NBT groups. A FEMS table including a motif not having such a difference may act as noise that only increases the amount of computation of the model without providing information essential for classification. Therefore, in order to exclude these meaningless motifs, only specific motifs having significant relative frequency differences between the three groups were selected.

In addition, in order to prevent the model overfitting issue in the size and motif selection process, only the training set was used in the size and motif selection process.

That is, the nucleic acid fragment end motifs were set with 4 bases (A, T, G, C) using the NGS data generated in Example 1 and some motifs that had statistically significant (Kruskal-Wallis Test, FDR-adjust p<0.05) relative frequency difference between healthy subjects (Normal) and neuroblastoma (NBT) patient groups were selected from a total of 256 types (4*4*4*4) of motifs (FIG. 3).

In addition, motifs having a mean frequency higher than the random baseline (1/256, 0.004) were further selected from the motifs selected through the above process in the healthy subject group in order to prevent overfitting.

As a result, a total of 84 motifs were obtained and detailed motif information is as follows:

CTGG, ACTT, CCTA, TGGA, TGGG, CAGG, TATA, CCTT, CAGC,
TAGA, AGAA, AGAG, CATA, CAGT, CAGA, ACCT, CTGT, ACAT, GCTT,
GCTA, TCAG, CTTA, GGCC, ATTT, CCCA, TATC, CCTG, TCTA, GCCT,
ACTG, TGAG, GGTA, CATT, TATT, CCAT, CCTC, CCAA, CTTT, TAAG,
GCTG, CCCT, TGAA, ACCA, GTTT, TGTA, CTCA, GCCA, TATG, GCAT,
AAAG, AAAA, GGCT, TGAC, AGCA, TCTT, CTGA, CATC, ACAA, GACA,
AACA, CCCC, CACT, GGAG, GGCA, TCAA, CAAG, TAAA, AAAT, TGCC,
GGTT, GGGA, CCAC, TGTG, CATG, TGCA, GAAT, TGTC, TGCT, CAAT,
GGAA, AGTG, TACT, CACA, TCCC

### 6-2. Nucleic Acid Fragment Size Selection

Most of the nucleic acid fragments whose quality has been determined have a size in the range of 90 to 250, as shown in FIG. 3. Therefore, when a FEMS table includes an area that is out of this size range, most areas are filled with zero (0) and only meaningless noise increases. For this reason, the nucleic acid fragment size was selected within this range.

### Example 7. Production of Fragment End Motif Frequency and Size (FEMS) Table and FEMS_Z Table

### 7-1 Production of FEMS Table

Two-dimensional vectors were generated by plotting motif types on the X-axis and fragment sizes on the Y-axis to simultaneously express the end motif frequency and size information of the nucleic acid fragments selected in Example 6. More specifically, as shown in the left panel of FIG. 4, the type and size of nucleic acid motifs at both ends of one nucleic acid fragment are expressed as a frequency, and this is extended to the entire nucleic acid fragment and accumulated, to generate two-dimensional vectors as shown in FIG. 4.

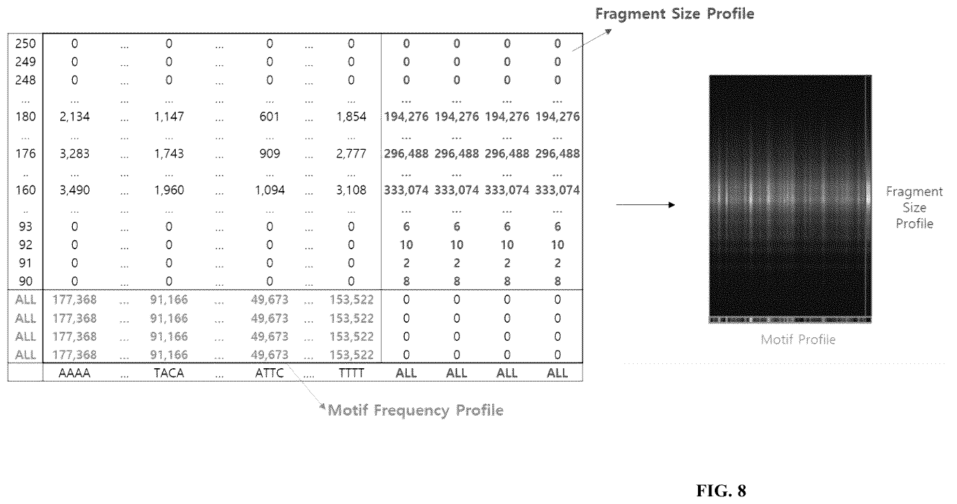

Also, an edge summary was further performed by adding a column sum four times to the bottom of the two-dimensional vector in order to add frequency information for each fragment end motif irrelevant to the fragment size, and adding a row sum four times to the rightmost part of the two-dimensional vector in order to add the fragment size information irrelevant to the fragment end motif, to generate a two-dimensional vector as shown in the left panel of FIG. 5. The two-dimensional vector is defined as a fragment end motif frequency and size (FEMS) table. The FEMS table was visualized and an example thereof is shown in FIG. 8.

### 7-2 Production of FEMS_Z Table

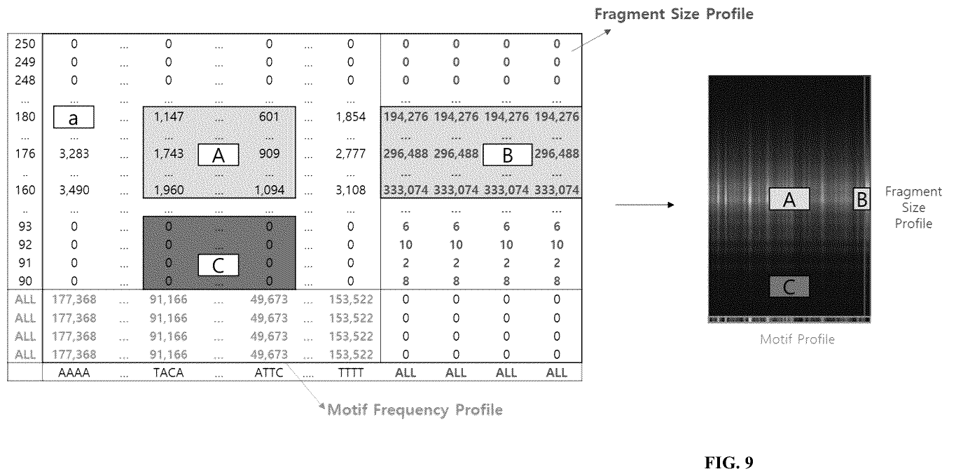

The values constituting the FEMS table formed in 7-1 mean the frequencies of nucleic acid fragments with specific sizes and motifs. As shown in FIG. 9, this frequency value is characterized in that there is a large difference in the distribution between values calculated in relatively high-frequency regions (A and B) and low-frequency regions (C). For example, a difference of 100 units is observed in region A, a difference of 10,000 units is observed in region B, whereas a difference of only 1 unit is rarely observed in region C. When this FEMS table is used, there is a problem in that it becomes difficult for CNN-based AI algorithms to learn parameters (weights). Therefore, additional pretreatment was performed to have values in a similar range in all areas of the FEMS table to create the FEMS_Z.

Specifically, 99 healthy subjects included in the training data in Table 4 were selected as a Z reference set and means and standard deviations observed at each position in the FEMS table in the selected Z reference set were calculated.

For example, the mean and standard deviation of values at the position (a) having a nucleic acid fragment size of 180 and having an AAAA motif were calculated in the FEMS table of the Z-reference group of 99 subjects, and defined as Mean_180_AAAA and SD_180_AAAA, respectively.

Z standardization was performed using the mean and standard deviation at each position in the FEMS table calculated in the above process. Specifically, the frequency value observed at the position having a nucleic acid fragment size of 180 and the AAAA motif is defined as Value 180 AAAA, Z standardization was performed in accordance with the equation of Z_180_AAAA=(Value_180_AAAA−Mean_180_AAAA)/SD_180_AAAA (FIG. 10).

In order to avoid the influence of Z standardization values that do not fall within the normal range (−5 to 5) due to the excessively small standard deviations, the minimum and maximum ranges of Z standardization values were limited to −5 for Z<−5 and 5 for Z>5.

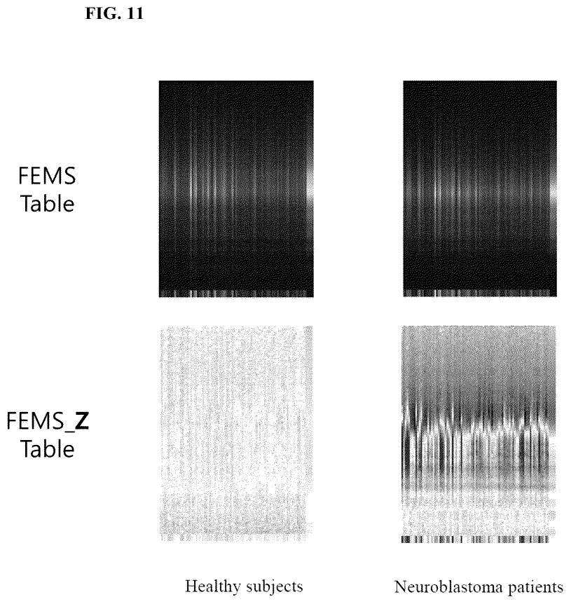

In the above process, 2D vectors obtained by substituting values of all positions in the conventional FEMS table with Z-standardized values were defined in the FEMS_Z table, and a visual comparison of the FEMS table with the FEMS_Z table is shown in FIG. 11.

The FEMX_Z table was formed using an edge summary including adding the column sum 4 times to the bottom of the 2D vector in order to add frequency information for each fragment end motif regardless of fragment size and adding the row sum 4 times to the rightmost side of the 2D vector in order to add fragment size information regardless of fragment end motif.

### Example 8. Second CNN Model Construction and Training Process

A CNN artificial intelligence model that distinguished healthy subjects from neuroblastoma cancer patients was trained using the FEMS table or FEMS_Z table two-dimensional vector as an input.

The datasets of Table 4 were used and the training dataset was used for model training, the validation dataset was used for hyper-parameter tuning, and the test dataset was used for final model testing.

The basic configuration of the CNN model is shown in FIG. 11. A sigmoid was used as an activation function, three convolution layers were used, and 13 10*10 patches were used. For the pooling method, a max mode and a 2×2 patch were used. Four fully connected layers were used and 454 hidden nodes were included. Finally, the final DPI was calculated using the sigmoid function value.

Hyper-parameter tuning is a process of optimizing the values of various parameters (the number of convolution layers, the number of dense layers, the number of convolution filters, etc.) constituting the CNN model. The hyper-parameter tuning was performed using Bayesian optimization and grid search techniques. When the validation loss started to increase compared to training loss, it was considered that the model was overfitting and model training was stopped.

The performance of several models obtained through hyper-parameter tuning was compared using the validation dataset, the model having the best performance of the validation dataset was determined as the optimal model, and final performance evaluation was performed with the test dataset.

When the FEMS_Z table 2D vector of a random sample was input to the model created through the above process, the probability that the sample is a healthy subject, and the probability that the sample is an ovarian cancer patient were calculated through the sigmoid function, which is the last layer of the CNN model. Such probability was defined as “deep probability index (DPI)”.

### Example 4. Evaluation of Performance of Second Artificial Intelligence Model

### 9-1 Evaluation of Performance (Test)

The performance of the DPI output from the FEMS deep learning model and the FEMS-Z deep learning model constructed in Example 8 was tested. All samples were divided into training, validation, and test groups. The models were constructed using the training samples, and then the performance of the models constructed using the training samples was evaluated using the samples of the validation and test groups.

TABLE 5

| | Accuracy | | F1-score | | Precision | | AUC | |
|---|---|---|---|---|---|---|---|---|
| | FEMS | FEMS_Z | FEMS | FEMS_Z | FEMS | FEMS_Z | FEMS | FEMS_Z |
| Train | 1.000 | 1.000 | 1.000 | 1.000 | 1.000 | 1.000 | 1.000 | 1.000 |
| Validation | 1.000 | 1.000 | 1.000 | 1.000 | 1.000 | 1.000 | 1.000 | 1.000 |
| Test | 0.987 | 1.000 | 0.973 | 1.000 | 0.947 | 1.000 | 1.000 | 1.000 |

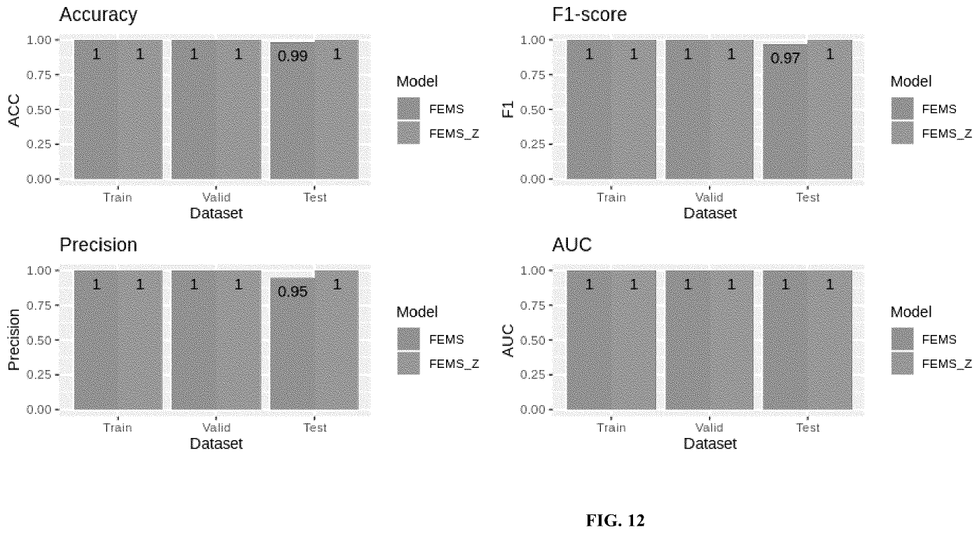

As a result, as can be seen from Table 5 and FIG. 12, accuracy for Train, Valid, and Test groups was 100%, 98.1%, and 91.1% for the FEMS model, respectively, and was 100%, 100%, and 100% for the FEMS_Z model. In addition, it can be seen that the performance of the model trained with the FEMS_Z table as an input is excellent in all of F1-score, precision and AUC.

### 9-2. DPI Distribution

How much the DPI, which is the output value of the deep learning model constructed in Example 9-1, matched the actual patient was determined.

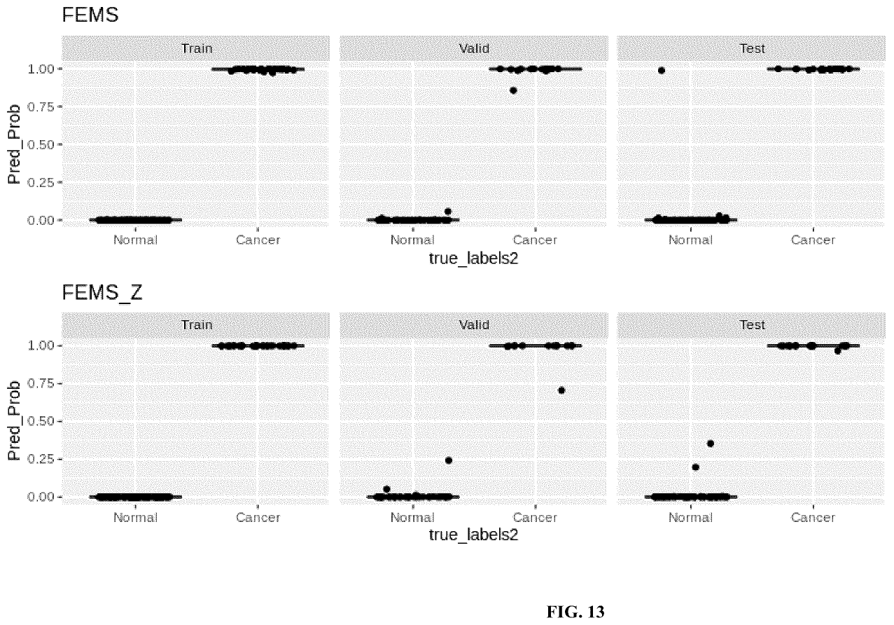

As a result, as can be seen from FIG. 13, the FEMS_Z table learning model had a higher probability of classifying normal samples as normal subjects and neuroblastoma samples as neuroblastoma patients than the FEMS table learning model.

### Example 10. Construction of Ensemble Model of First Artificial Intelligence Model and Second Artificial Intelligence Model and Performance Verification

### 10-1. Reconstruction of First Artificial Intelligence Model

A multilayer perceptron (MLP) model was designed using the features extracted in Example 2 from the sample data in Table 4 and was reconstructed to output DPI values.

### 10-2. Construction of Ensemble Models

An ensemble model that outputs, as the final result value (EPI, ensemble probability index), the mean of the output value of the second artificial intelligence model of Example 7 and the output value of the first artificial intelligence model of Example 10-1 was constructed.

### 10-3. Comparison of Performance Between Ensemble Model and Each Model

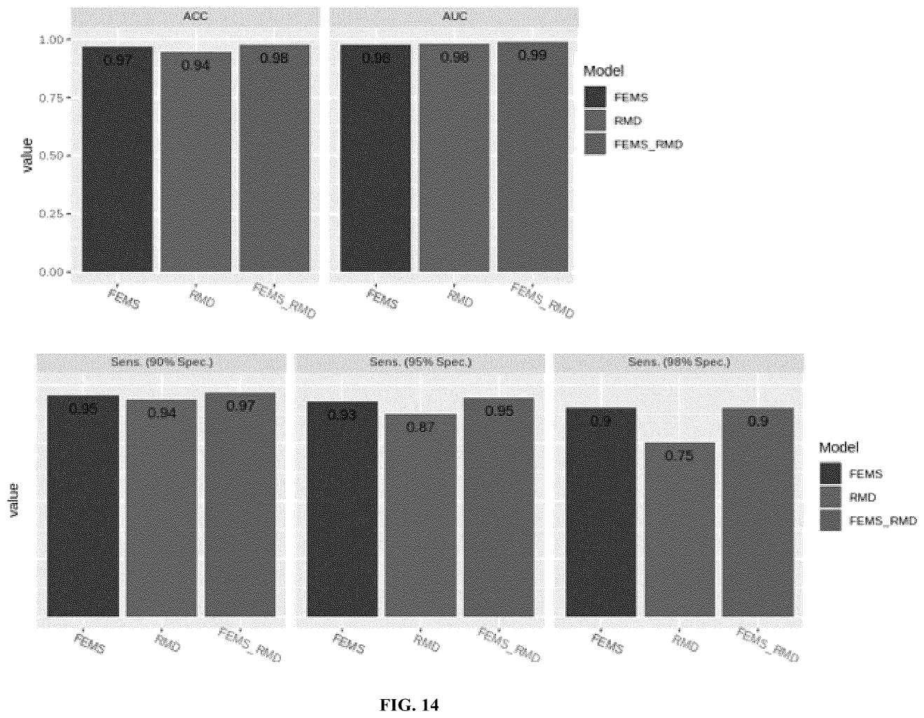

The performance was compared between the second artificial intelligence model of Example 7, the reconstructed first artificial intelligence model of Example 10-1, and the ensemble model of Example 10-2. As shown in FIG. 14, the result showed that the performance of the ensemble model was the most outstanding.

Although specific configurations of the present invention have been described in detail, those skilled in the art will appreciate that this detailed description is provided as preferred embodiments for illustrative purposes and should not be construed as limiting the scope of the present invention. Therefore, the substantial scope of the present invention is defined by the accompanying filed claims and equivalents thereto.

What is claimed is:

1. A method for diagnosing cancer and predicting a cancer type, the method comprising:
(a) obtaining a sequence information by extracting nucleic acids from a biological sample;
(b) aligning the sequence information (reads) with a reference genome database;
(c) dividing the reference genome into predetermined bins;
(d) obtaining two or more pieces of information selected from the group consisting of cancer-specific single nucleotide variant distribution (regional mutation density, RMD) information, cancer-specific single nucleotide variant frequency (mutation signature) information, end sequence motif frequency information of nucleic acid fragments, and size information of nucleic acid fragments using the aligned reads in predetermined bins;
(e) obtaining an output value by inputting the two or more pieces of information to an artificial intelligence model trained to perform cancer diagnosis and cancer type prediction and analyzing the same;
(f) determining whether or not cancer develops by comparing the analyzed output value with a cut-off value; and
(g) predicting a cancer type through comparison of the output value.

2. The method according to claim 1, wherein the bin in step (c) has a size of 100 kb to 10 Mb.

3. The method according to claim 1, wherein the cancer-specific single nucleotide variant in step (d) is obtained by detecting single nucleotide variants, followed by filtering and extraction.

4. The method according to claim 1, wherein the calculating the cancer-specific single nucleotide variant distribution (regional mutation density, RMD) information in step (d) is performed by a method comprising the following steps:
(i) calculating the number of single nucleotide variants extracted for each of bins excluding bins in which no variants are detected above the cut-off value of the entire sample; and
(ii) dividing the calculated number by a total number of variants for each bin, following by normalization.

5. The method according to claim 1, wherein the frequency of the end sequence motifs of the nucleic acid fragments in step (d) corresponds to the number of motifs detected in all the nucleic acid fragments.

6. The method according to claim 1, wherein the size of the nucleic acid fragment in step (d) corresponds to the number of bases from a 5′ end to a 3′ end of the nucleic acid fragment.

7. The method according to claim 1, further comprising the following steps, after step (d) and before step (e) of inputting the information to the artificial intelligence model:
(i) generating vectorized data using the end sequence motif frequency information and size information of the nucleic acid fragments; and
(ii) post-processing the vectorized data.

8. The method according to claim 1, wherein the two or more pieces of information in step (e) comprise cancer-specific single nucleotide variant distribution (regional mutation density, RMD) information and cancer-specific single nucleotide variant frequency (mutation signature) information, or sequence motif frequency information of nucleic acid fragments and size information of nucleic acid fragments, or cancer-specific single nucleotide variant distribution (regional mutation density, RMD) information, cancer-specific single nucleotide variant frequency (mutation signature) information, sequence motif frequency information of nucleic acid fragments, and size information of nucleic acid fragments.

9. The method according to claim 1, wherein the artificial intelligence model of step (e) comprises two or more modules configured to analyze the input two or more pieces of information and output the resulting values.

10. The method according to claim 9, wherein the module is selected from the group consisting of K-nearest neighbors, linear regression, logistic regression, support vector machine (SVM), decision trees, random forests, and artificial neural network.

11. The method according to claim 9, wherein the artificial intelligence model further comprises an output module configured to collect and analyze result values output from each module thereby to output a final result value.

12. The method according to claim 11, wherein the output module outputs, as the result value, at least one selected from the group consisting of a sum, difference, product, mean, logarithm of the product, logarithm of the sum, median, quantile, minimum, maximum, variance, standard deviation, median absolute deviation, and coefficient of variance of a result value output by each module itself or a weighted value thereof.

13. The method according to claim 11, wherein the output module is an ensemble model selected from the group consisting of voting, bagging, boosting, and stacking.

14. The method according to claim 13, wherein the boosting model is selected from the group consisting of AdaBoost (adaptive boosting), GBM (gradient boosting machine), XGBoost (extra gradient boost) and LightGBM (light gradient boost).

15. The method according to claim 10, wherein the artificial neural network is selected from the group consisting of a convolutional neural network (CNN), a deep neural network (DNN), a recurrent neural network (RNN), and an autoencoder.

16. The method according to claim 1, wherein the output value of step (e) is a deep probability index (DPI).

17. The method according to claim 1, wherein the cut-off value in step (f) is 0.5 and a determination is made that cancer has developed when the output value is 0.5 or more.

18. The method according to claim 1, wherein the step (g) of predicting the cancer type through comparison of the output values comprises determining the cancer type showing a highest value among output result values as the cancer type of the sample.

19. A device for diagnosing cancer and predicting a cancer type, the device comprising:
a decoder configured to extract nucleic acids from a biological sample and decode sequence information;
an aligner configured to align the decoded sequence with a reference genome database;
an input information receiver configured to divide the reference genome into predetermined bins and obtain two or more pieces of information selected from the group consisting of cancer-specific single nucleotide variant distribution (regional mutation density, RMD) information, cancer-specific single nucleotide variant frequency (mutation signature) information, end sequence motif frequency information of nucleic acid fragments, and size information of nucleic acid fragments using the aligned reads in each of predetermined bins;
an artificial intelligence model analyzer configured to input the two or more pieces of information to an artificial intelligence model trained to perform cancer diagnosis and cancer type prediction and analyze the information to obtain an output value;
a cancer diagnostic unit configured to compare the output value with a cut-off value to determine whether or not cancer develops; and
a cancer type predictor configured to predict a cancer type through comparison of the output values.

20. A computer-readable storage medium including an instruction configured to be executed by a processor for diagnosing cancer and predicting a cancer type through the following steps, comprising:
(a) obtaining a sequence information by extracting nucleic acids from a biological sample;
(b) aligning the sequence information (reads) with a reference genome database;
(c) dividing the reference genome into predetermined bins;
(d) obtaining two or more pieces of information selected from the group consisting of cancer-specific single nucleotide variant distribution (regional mutation density, RMD) information, cancer-specific single nucleotide variant frequency (mutation signature) information, end sequence motif frequency information of nucleic acid fragments, and size information of nucleic acid fragments using the aligned reads in predetermined bins;
(e) obtaining an output value by inputting the two or more pieces of information to an artificial intelligence model trained to perform cancer diagnosis and cancer type prediction and analyzing the same;
(f) determining whether or not cancer develops by comparing the analyzed output value with a cut-off value; and
(g) predicting a cancer type through comparison of the output value.
